(19) United States
(12) Patent Application Publication
(10) Pub. No.: US 2023/0126447 A1
(43) Pub. Date: Apr. 27, 2023

(54) OCULAR DRUG DEPOT FOR COMPLEMENT-MEDIATED DISORDERS

(71) Applicant: Achillion Pharmaceuticals, Inc., Blue Bell,, PA (US)

(72) Inventors: Mingjun Huang, New Haven, CT (US); Steven Zelenkofske, Center Valley, PA (US); David Boyer, New Haven, CT (US); Jose Rivera, Berlin, CT (US); Jason Allan Wiles, Madison, CT (US)

(73) Assignee: Achillion Pharmaceuticals, Inc., Boston, MA (US)

(21) Appl. No.: 17/909,228

(22) PCT Filed: Mar. 9, 2021
(86) PCT No.: PCT/US2021/021563

§ 371 Date: Sep. 2, 2022

Related U.S. Application Data
(60) Provisional application No. 62/987,777, filed on Mar. 10, 2020.

(51) Int. Cl. A61K 31/506; A61K 45/06; A61P 27/02

(52) U.S. Cl. CPC A61K 31/506; A61K 45/06; A61P 27/02

(57) ABSTRACT

This disclosure provides an ocular tissue depot of a selected complement factor D (CFD) inhibitor for the extended treatment of an ocular disorder mediated by complement factor D. In particular, the disclosure includes the creation of a drug depot within the choroid-retina pigmented epithelium (C-RPE) and/or the iris-ciliary body (I-CB) of the eye by means of the systemic (oral or parenteral) administration of the compound, where the compound accumulates in ocular tissue and is released over an extended period for the treatment of a posterior and/or anterior eye disorder.

### CROSS-REFERENCE TO RELATED APPLICATIONS

This application claims the benefit of priority to U.S. Application No. 62/987,777, filed Mar. 10, 2020, which is incorporated by reference herein for all purposes.

### FIELD OF THE DISCLOSURE

This disclosure provides an ocular tissue depot of a selected complement factor D (CFD) inhibitor for the extended treatment of an ocular disorder mediated by complement factor D. In particular, embodiments include the creation of a drug depot within the ocular tissue, i.e., the choroid-retina pigmented epithelium (C-RPE) and/or the iris-ciliary body (I-CB) of the eye by means of the systemic (oral or parenteral) administration of at least one compound, where the compound accumulates in ocular tissue and is released over an extended period for the treatment of a posterior and/or anterior eye disorder.

### BACKGROUND

The complement system is a key component of innate immunity. It consists of a large group of plasma and membrane bound proteins that play a central role in the defense against infection and in the modulation of immune and inflammatory responses. The complement system can be activated via three distinct pathways, namely, the classical, the alternative and the lectin pathways. Complement activation triggers a sequence of biological reactions. The classical pathway can be activated by immune complexes or by substances such as C-reactive protein, and the complement components involved include C1, C2, C4 and C3. The alternative pathway provides a rapid, antibody-independent route of complement activation and amplification. The alternative pathway directly activates C3 when it interacts with certain activating surfaces (e.g., zymosan, lipopolysaccharides) and involves the actions of C3, Factor B, Factor D, and properdin. The activation of the lectin pathway is also independent of immune complex generation, and can be achieved by interaction of certain serum lectins, such as mannose binding lectin (MBL), with mannose and N-acetyl glucosamine residues present in abundance in bacterial cell walls.

In the normal eye, the complement system is continuously activated at low levels and both membrane-bound and soluble intraocular complement regulatory proteins tightly regulate this spontaneous complement activation. This allows protection against pathogens without causing any damage to self-tissue and vision loss. Activated complement, however, has the potential to inflict damage to self-tissue. The presence and activation of complement has been suggested to play a crucial role in the pathogenesis of a large number of diseases, including ocular diseases (See, e.g., Thurman J M, Holers V M. The central role of the alternative complement pathway in human disease. J. Immunol. 2006; 176:1305-1310).

The alternative complement system has been implicated in a large number of ocular disorders that may affect the anterior region of the eye, the posterior region of the eye or the whole eye. One notable ocular disorder in which complement has been implicated is age-related macular degeneration (AMD), which is a leading cause of vision loss in industrialized countries. There are two main types of AMD; the dry (non-neovascular) form and the wet (neovascular) form. Geographic atrophy (GA) is a chronic progressive degeneration of the macula, and is considered as part of late-stage age-related macular degeneration (AMD). The condition leads to central scotomas and permanent loss of visual acuity. The disease is characterized by localized, sharply demarcated atrophy of outer retinal tissue, retinal pigment epithelium and choriocapillaris. The alternative complement system has also been implicated in diabetic retinopathy and diabetic macular edema (DME). These conditions can lead to the loss of vision over time, and diabetic retinopathy is the largest contributor to vision loss in the United States. Additional complement-mediated disorders include retinal vein occlusion (RVO).

Commercial treatment options for AMD include anti-angiogenic drugs and vascular endothelial growth factor (VEGF) inhibitors, such as the USFDA approved brolucizumab-dbll (Beovu), pegaptanib sodium (Macugen), ranibizumab (Eylea and Lucentis), and bevacizumab (Avastin).

Administration of drugs to the eye present challenges. Local administration is possible via intravitreal or posterior injections, which may be uncomfortable for the patient, and may require dual treatments if both eyes are affected. Anterior delivery is possible via eye drops, but only if they can effectively pass through the mucosa and are not blinked away. Systemic delivery exposes the entire body to the drug, even though therapy is only needed in the eye. These challenges are compounded by the fact that therapy may be needed over an extended period of time. New methods of administration of active compounds to the eye are desirable.

Patent filings that disclose complement factor D inhibitors are described in U.S. Pat. Nos. 9,598,446; 9,643,986; 9,663; 543; 9,695,205; 9,732,103; 9,732,104; 9,758,537; 9,796,741; 9,828,396; 10,000,516; 10,005,802; 10,011,612; 10,081,645; 10,087,203; 10,092; 584; 10,100,072; 10,138,225; 10,189,869; 10,106,563; 10,301,336; and 10,287,301; International Publication Nos. WO2019/028284; WO2018/160889; WO2018/160891; WO2018/160892; WO2017/035348; WO2017/035349; WO 2017/035351; WO 2017/035352; WO 2017/035353; WO 2017/035355; WO2017/035357; WO2017/035360; WO2017/035361; WO2017/035362; WO2017/035415; WO2017/035401; WO2017/035405; WO2017/035413; WO2017/035409; WO2017/035411; WO2017/035417; WO2017/035408 WO2015/130784; WO2015/130795; WO2015/130806; WO2015/130830; WO2015/130838; WO2015/130842; WO2015/130845; and WO2015/130854; and U.S. Patent Publication Nos. US 2016-0361329; US 2016-0362432; US 2016-0362433; US 2016-0362399; US 2017-0056428; US 2017-0057950; US 2017-0057993; US 2017-0189410; US 2017-0226142; US 2017-0260219; US 2017-0298084; US 2017-0298085; US 2018-0022766; US 2018-0022767; US 2018-0072762; US 2018-0030075; US 2018-0169109; US 2018-0177761; US 2018-0179185; US 2018-0179186; US 2018-0179236; US 2018-0186782; US 2018-0201580; US 2019-0031692; US 2019-0048033; US 2019-0144473; and US 2019-0211033; all owned by Achillion Pharmaceuticals, Inc, now Alexion Pharmaceuticals, Inc.

It is an object of the present disclosure to provide new treatment regimens for complement factor D-mediated ocular disorders.

### SUMMARY OF THE DISCLOSURE

Selected complement factor D inhibitor compounds accumulate disproportionately in ocular tissue over plasma and other tissues on systemic administration, which unexpectedly results in a depot of the compound in the choroid-retina pigmented epithelium (C-RPE, or choroidal tissue) and/or the iris-ciliary body (I-CB), without the required use of an injection or medical device. A method to treat an ocular disorder mediated by complement factor D is provided wherein a compound depot of the identified complement factor D inhibitor is created and provided based on systemic (i.e., oral or parenteral) delivery to the patient, for example a human, in need thereof, wherein the compound accumulates in the choroid-retina pigmented epithelium (C-RPE) and/or the iris-ciliary body (I-CB) of the eye and is available to provide extended therapy after cessation of the systemic delivery of the compound. In another embodiment, a method is provided that includes providing a systemic dosage that is less then what would be administered for a systemic complement factor D mediated disorder (for example PNH, which is Paroxysmal Nocturnal Hemoglobinuria, C3G or aHUS) but which is sufficient to form an accumulation in the choroid-retina pigmented epithelium (C-RPE) and/or the iris-ciliary body (I-CB) of the eye over time, which results in an advantageous ocular therapy with less toxicity.

The extended ocular release of the complement factor D compound can be confirmed by its therapeutic effect over time against the complement factor D ocular disorder being treated, after cessation of systemic treatment. The depot can also be confirmed using compound radiolabeling, as well known in the art and described below.

The accumulation of the complement factor D compound in the choroid-retina pigmented epithelium (C-RPE) and/or the iris-ciliary body (I-CB) of the eye can also be confirmed via the use of the well-accepted animal model of Dutch Belted Rabbits, as illustrated in the Examples and Tables herein. For example, when 15 mg/kg was administered a single time to Dutch Belted Rabbits, as shown in Example 3 and Table 7, at just one hour after administration, there was 318 ng/mL of Compound 1 in the plasma compared with 917 ng/g of Compound 1 in the C-RPE and 1010 ng/g of Compound 1 in the I-CB. Importantly, the levels of Compound 1 in the C-RPE and/or I-CB after only a single 15 mg/kg dose were still detectable at 240 hours (10 days) after administration, while no drug was detectable in the plasma at 96 hours. The accumulation observed in the C-RPE and the I-CB was also surprising in that the C-RPE and/or I-CB began to saturate its ability to retain Compound 1 over time. For example, as shown in Example 3, Table 8, and FIG. 7 at hour 1 of day 15 following twice a day administration of 15 mg/kg of Compound 1 to Dutch Belted Rabbits, Compound 1 showed some accumulation in the plasma (845 ng/ml), but significant accumulation in the C-RPE (4870 ng/g) and I-CB (4920 ng/g). At 240 hours after the last administration, the R-CPE and I-CB still had significant drug concentrations (C-RPE 340 ng/g; I-CB 408 ng/g), while the drug in the plasma measured only 3.19 ng/ml at 96 hours. Surprisingly, the concentration of Compound 1 in the retina (25.3 ng/g) was much less but still measurable at 240 hours (10 days), strongly indicating that the drug contained in the depot of the C-RPE and/or I-CB was able to continue to contribute to the concentration of drug observed in the retina. As shown in Example 4, Table 17, and FIG. 14, similar observations were observed for Compound 1 dosed at 50 mg/kg given twice a day, with a concentration at 240 hours after administration of the last dose in the C-RPE of 194 ng/g and in the I-CB of 1620 ng/g, while the concentration in the retina was 39.3 ng/g. As shown in the Examples 3 and 4 below, in both the 15 mg/kg and 50 mg/kg twice a day dosing cohorts, the concentration of Compound 1 contained in the C-RPE and the I-CB remained above plasma level concentrations throughout the measured time points by day 15, indicating the ability to provide sustained drug concentrations in particular areas of the eye without the requirement to achieve similar plasma concentrations.

The present disclosure thus provides a method for producing a depot for the delivery of an effective amount of an alternative pathway complement factor D (CFD) inhibitor of Formula I or Formula II, or a pharmaceutically acceptable salt thereof, optionally in a pharmaceutically acceptable carrier, for the treatment or prevention of a complement factor D-mediated disorder in the human eye without the use of an injection, implant, or medical device, wherein the CFD inhibitor is administered to the human via oral or parenteral delivery.

The present disclosure thus provides a method for treating an alternative pathway complement D-mediated ocular disorder in a subject in need thereof, the method including systemically administering to the subject a compound of Formula I, or a pharmaceutically acceptable salt thereof, wherein (i) the compound of Formula I has the structure:

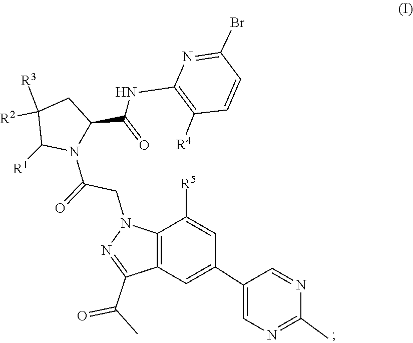

or an N-oxide, isotopic derivative, or prodrug thereof, or optionally in a pharmaceutically acceptable carrier to form a composition, wherein R¹ and R² are (a) both hydrogen; or (b) combined together to form a cyclopropyl ring; R³ is hydrogen, methyl, or fluoro; R⁴ is hydrogen or C₁-C₄ alkyl; and R⁵ is hydrogen or methyl; and (ii) a therapeutically effective amount of the compound of Formula I, or a pharmaceutically acceptable salt thereof, is systemically administered via oral or parenteral delivery to the subject. In some embodiments, the ocular disorder is a posterior ocular disorder. The posterior ocular disorders that can be treated using the methods of the inventions include those selected from the group consisting of dry and wet age-related macular degeneration (AMD), geographic atrophy, cytomegalovirus (CMV) infection, diabetic retinopathy, diabetic macular edema, choroidal neovascularization, acute macular neuroretinopathy, macular edema, Behcet's disease, retinal disorders, diabetic retinopathy, retinal arterial occlusive disease, central retinal vein occlusion, uveitic retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction, and retinitis pigmentosa. In certain embodiments, the posterior ocular disorder to be treated can be geographic atrophy, age-related macular degeneration (e.g., dry age-related macular degeneration or wet age-related macular degeneration), macular edema (e.g., cystoid macular edema or diabetic macular edema), diabetic retinopathy, or proliferative diabetic retinopathy. In other embodiments, the ocular disorder is an anterior ocular disorder (e.g., glaucoma, allergic conjunctivitis, anterior uveitis, or cataracts). In some embodiments, the systemic administration of the compound of the compound of Formula I, or a pharmaceutically acceptable salt thereof, results in an accumulation of the compound of Formula I in a melanin-containing ocular tissue of the subject, forming a depot that provides extended delivery of the compound of Formula I to an ocular tissue (e.g., choroid-retina pigmented epithelium (C-RPE) and/or iris ciliary body (I-CB)) of the subject for at least one week, two weeks, or three weeks after the administration. For example, The compound of Formula I can accumulate at a ratio of at least 2× in the choroidal or iris-ciliary body tissue of the eye versus in the plasma such that a depot is formed in the choroidal or iris-ciliary body tissue of the subject which provides extended delivery of the compound of Formula I, or a pharmaceutically acceptable salt thereof, for at least 7 days, two weeks, three weeks or one month, two months, three months, four months, five months, or six months after cessation of administration of the compound of Formula I, or a pharmaceutically acceptable salt thereof. In particular embodiments, the compound of Formula I, or a pharmaceutically acceptable salt thereof, is administered orally or parenterally once or twice a day. For example, the compound of Formula I, or a pharmaceutically acceptable salt thereof, can be administered orally once or twice a day in a tablet, capsule, gel cap, or other solid dosage form.

The present disclosure thus provides a method for treating an alternative pathway complement D-mediated ocular disorder in a subject in need thereof, the method including systemically administering to the subject a compound of Formula II, or a pharmaceutically acceptable salt thereof, wherein (i) the compound of Formula II has the structure:

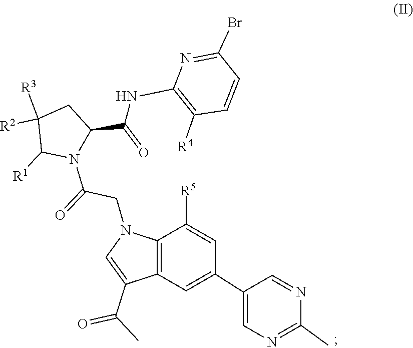

or an N-oxide, isotopic derivative, or prodrug thereof, or optionally in a pharmaceutically acceptable carrier to form a composition; wherein R¹ and R² are (a) both hydrogen; or (b) combined together to form a cyclopropyl ring; R³ is hydrogen, methyl, or fluoro; R⁴ is hydrogen or C₁-C₄ alkyl; and R⁵ is hydrogen or methyl; and (ii) a therapeutically effective amount of the compound of Formula II, or a pharmaceutically acceptable salt thereof, is systemically administered via oral or parenteral delivery to the subject. In some embodiments, the ocular disorder is a posterior ocular disorder. The posterior ocular disorders that can be treated using the methods of the inventions include those selected from the group consisting of dry and wet age-related macular degeneration (AMD), geographic atrophy, cytomegalovirus (CMV) infection, diabetic retinopathy, diabetic macular edema, choroidal neovascularization, acute macular neuroretinopathy, macular edema, Behcet's disease, retinal disorders, diabetic retinopathy, retinal arterial occlusive disease, central retinal vein occlusion, uveitic retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction, and retinitis pigmentosa. In certain embodiments, the posterior ocular disorder to be treated can be geographic atrophy, age-related macular degeneration (e.g., dry age-related macular degeneration or wet age-related macular degeneration), macular edema (e.g., cystoid macular edema or diabetic macular edema), diabetic retinopathy, or proliferative diabetic retinopathy. In other embodiments, the ocular disorder is an anterior ocular disorder (e.g., glaucoma, allergic conjunctivitis, anterior uveitis, or cataracts). In some embodiments, the systemic administration of the compound of the compound of Formula II, or a pharmaceutically acceptable salt thereof, results in an accumulation of the compound of Formula II in a melanin-containing ocular tissue of the subject, forming a depot that provides extended delivery of the compound of Formula II to an ocular tissue (e.g., choroid-retina pigmented epithelium (C-RPE) and/or iris ciliary body (I-CB)) of the subject for at least one week, two weeks, or three weeks after the administration. For example, The compound of Formula II can accumulate at a ratio of at least 2× in the choroidal or iris-ciliary body tissue of the eye versus in the plasma such that a depot is formed in the choroidal or iris-ciliary body tissue of the subject which provides extended delivery of the compound of Formula II, or a pharmaceutically acceptable salt thereof, for at least 7 days, two weeks, three weeks, or one month, two months, three months, four months, five months, or six months after cessation of administration of the compound of Formula II, or a pharmaceutically acceptable salt thereof. In particular embodiments, the compound of Formula II, or a pharmaceutically acceptable salt thereof, is administered orally or parenterally once or twice a day. For example, the compound of Formula II, or a pharmaceutically acceptable salt thereof, can be administered orally once or twice a day in a tablet, capsule; gel cap, or other solid dosage form.

As described below, it has been surprisingly discovered that selected compounds of Formula I and Formula II are capable of forming a depot within the choroid-retina pigmented epithelium (C-RPE) and/or the iris-ciliary body (I-CB) of the eye, which in certain embodiments may become saturated through oral or parenteral administration relatively quickly, for example within about 8 days. This accumulation or even saturated depot provides a drug concentration in the C-RPE and/or I-CB that is disproportionately greater than the drug concentration found in the plasma throughout the administration period (when comparing ng/g in ocular tissue to ng/mL in the plasma), for example greater than 2× or more, and typically significantly more than 2×, maintains a lower but potentially significant drug concentrations in the retina. By establishing a depot capable of delivering an effective amount of a CFD inhibitor, complement-mediated disorders affecting either an anterior or posterior area of the eye can be given extended therapy without the need for the creation of the depot through intravitreal and/or suprachoroidal injections or a medical device. The depot formed provides a sustained release from the C-RPE and/or the I-CB, and in some embodiments, saturation of the tissue, which allow for effective CFD inhibitor drug concentrations that persist after the cessation of drug administrations, and provides extended delivery to the eye for at least 7 days after cessation of administration.

In some embodiments, Formula I is selected from Compound 1 (danicopan) and Compound 2, wherein Compound 1 and Compound 2 have the structures:

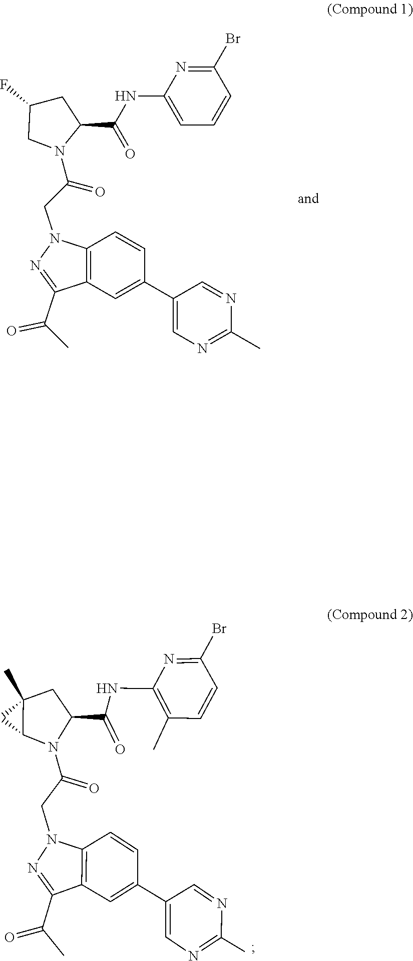 [SMILES: CC(=O)c1nn(CC(=O)N2C[C@H](F)C[C@H]2C(=O)Nc2cccc(Br)n2)c2ccc(-c3cnc(C)nc3)cc12 ; CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, Formula II is Compound 3 having the structure:

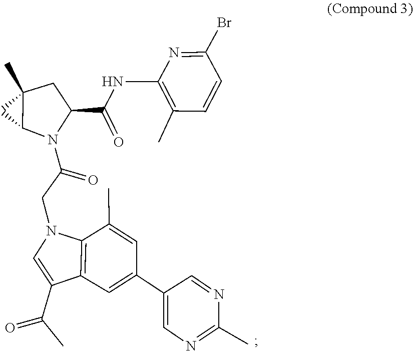 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

As demonstrated in the Examples, the depot formed in the C-RPE and/or I-CB provides a concentration of drug in those areas that provides for an extended exposure that is significantly greater than the residence time of the drug in the plasma. Accordingly, the drug concentrations achievable following depot accumulation in the C-RPE and/or I-CB greatly exceed those found in the plasma. For example, C-REP drug concentrations for Compound 1 in rabbit eyes were between 2× (50mgBID; at day 15; hour 1) and 350× (15mgBID; day 15; hour 24) greater than plasma drug concentrations following a dosing regimen believed to result in depot accumulation, and in some embodiments, saturation. Furthermore, significant drug concentrations (196 ng/ml at 50 mg BID; 340 ng/ml at 15 mg BID) were observed for at least 10 days following the last oral administration of the drug. Disproportionate drug concentrations were also seen in the I-CB compared to plasma, and also persisted for at least 10 days following the last oral administration of the drug. Importantly, the drug depot of the C-RPE and/or I-CB were apparently able to continue to contribute to the drug which accumulated in the retina, including at least 10 days following the last oral administration of the drug.

The depot comprising a compound of Formula I or II can be established via oral or parenteral administration. In some embodiments, the depot is established by oral or parenteral administration once a day. In some embodiments, the depot is established by oral or parenteral administration once a day, wherein the depot reaches significant accumulation, and in some embodiments near saturation, within about 15 days, 12 days, 10 days, 8 days, 3 days, or less. In some embodiments, the depot is established by oral administered no more than once a day. In some embodiments, the administration regimen includes a ramp-up or bolus dosage for a first period of time, for example, until depot accumulation, and in some embodiments, saturation is achieved, and then a lower or less frequent maintenance dosage for a second period of time. In some embodiments, following the sufficient accumulation of drug in the depot, the CFD inhibitor is administered every other day, bi-weekly, or once weekly.

In some embodiments, the oral or parenteral administration regimen results in a depot that provides extended delivery of the CFD inhibitor for at least one, two, three, four, five, or six months after cessation of administration the CFD inhibitor.

In some embodiments, the oral or parenteral administration regimen results in a C-RPE and/or I-CB depot having a drug concentration that is at least 2× greater, 3×, greater, 4× greater, or 5× greater than the drug concentration in the plasma across the administration period. In some embodiments, the oral or parenteral administration regimen results in a retina drug concentration that is no less than 0.6×, 0.65×, 0.7×, 0.75× or greater than the drug concentration in the plasma across the administration period. Methods of measuring the concentration of a drug in the plasma and in the various compartments of the eye are well known, and described further below.

In some embodiments, the administration regimen of the present disclosure comprises the oral administration of Compound 1, wherein Compound 1 is administered in a dosing regimen resulting in an AUC₀₋₂₄ of between about 2500 ng*hr/ml and 12,000 ng*hr/ml. In some embodiments, the AUC₀₋₂₄ of Compound 1 is between about 2500 ng*hr/ml and 10,000 ng*hr/ml. In some embodiments, the AUC₀₋₂₄ of Compound 1 is less than 10,000 ng*hr/ml. In certain embodiments, the method is characterized by (a) the dosing regimen provides a plasma AUC₀₋₂₄ of between about 2500 ng*hr/mL and about 12000 ng*hr/mL; (b) the dosing regimen results in a depot of Compound 1 within an eye of the subject by day 15 of administration, and (c) by day 15, the ratio of Compound 1 in the choroid-retina pigmented epithelium or iris-ciliary body of the eye compared to the plasma is maintained at no less than 2.5× throughout the dosing period. In some embodiments, the dosing regimen results in a depot of Compound 1 within an eye by day 8, by day 5, or by day 3 of administration. In particular embodiments, the ratio of Compound 1 in the choroid-retina pigmented epithelium or of the eye compared to the plasma is maintained between a range of 2.75 to 5.75 or greater throughout the dosing period. In certain embodiments, the ratio of Compound 1 in the iris-ciliary body compared to the plasma is maintained between a range of 3 to 5.5 or greater throughout the dosing period. In some embodiments, Compound 1 is administered once a day. In some embodiments, Compound 1 is administered once a day in a dose of between about 200 mg and 1000 mg, or twice a day in a dose of about 50 mg to 250 mg (e.g., about 100 mg, about 200 mg, or about 250 mg). In some embodiments, Compound 1 is administered once a day in a dose of between about 200 mg and 800 mg. In some embodiments, Compound 1 is administered in a dose of less than 800 mg. In some embodiments, Compound 1 is administered in a dose of about 400 mg once a day. In some embodiments, Compound 1 is administered in a dose of about 100 mg to about 200 mg twice a day.

In some embodiments, the administration regimen of the present disclosure comprises the oral administration of Compound 2, wherein Compound 2 is administered in a dosing regimen resulting in an AUC₍₀₋₂₄₎ of between about 500 ng*hr/ml and 4,500 ng*hr/ml. In some embodiments, Compound 2 is administered in a dosing regimen resulting in an AUC₍₀₋₂₄₎ of between about 500 ng*hr/mL and 3,000 ng*hr/mL. In some embodiments, Compound 2 is administered in a dosing regimen resulting in an AUC₍₀₋₂₄₎ of less than 2,500 ng*hr/mL. In some embodiments, Compound 2 is administered in a dosing regimen resulting in an AUC₍₀₋₂₄₎ of less than 1,000 ng*hr/mL. In certain embodiments, the method is characterized by (a) the dosing regimen provides a plasma AUC₀₋₂₄ of between about 500 ng*hr/ml and 4,500 ng*hr/ml; (b) the dosing regimen results in a depot of Compound 2 within an eye of the subject by day 15 of administration, and (c) by day 15, the ratio of a compound of Compound 2 in the retina of the eye compared to the plasma of the subject is maintained at no less than 0.60 throughout the dosing period. In other embodiments, the method is characterized by (a) the dosing regimen provides a plasma AUC₀₋₂₄ of between about 500 ng*hr/ml and 4,500 ng*hr/ml; (b) the dosing regimen results in a depot of Compound 2 within an eye of the subject by day 15 of administration, and (c) by day 15, the ratio of Compound 2 in the choroid-retina pigmented epithelium of the eye compared to the plasma is maintained at no less than 2.5× throughout the dosing period. In certain embodiments, the method is characterized by (a) the dosing regimen provides a plasma AUC₀₋₂₄ of between about 500 ng*hr/ml and 4,500 ng*hr/ml; (b) wherein the dosing regimen results in a depot of Compound 2 within an eye of the subject by day 15 of administration; and (c) by day 15, the ratio of Compound 2 in the iris-ciliary body of the eye compared to the plasma of the subject is maintained at no less than 3 throughout the dosing period. In some embodiments, the dosing regimen results in a depot of Compound 2 in an eye of the subject by day 8, day 5, or day 3 of administration. In particular embodiments, the ratio of Compound 2 in the retina of the eye compared to the plasma of the subject is maintained at no less than 0.65, no less than 0.70, or no less than 0.75 throughout the dosing period. In particular embodiments, the ratio of Compound 2 in the choroid-retina pigmented epithelium of the eye compared to the plasma is maintained between a range of 2.75 to 5.75 or greater throughout the dosing period and/or the ratio of Compound 2 in the ins-ciliary body compared to the plasma is maintained between a range of 3 to 5.5 or greater throughout the dosing period. In particular embodiments, the method includes orally administering Compound 2, or a pharmaceutically acceptable salt thereof, at a dosing regimen that provides a plasma AUC₀₋₂₄ of between about 500 ng*hr/ml and 4,500 ng*hr/ml; wherein the dosing regimen results in a depot of Compound 2 within an eye of the subject by day 15, by day 8, or by day 5 of administration, and wherein the depot contributes to the effective amount of Compound 2 contained in the eye throughout the dosing period. In some embodiments, Compound 2 is administered in a dose of between about 40 mg and 300 mg. In some embodiments, Compound 2 is administered once a day or twice a day. In some embodiments, Compound 2 is administered once a day in a dose of between about 80 mg and 250 mg. In some embodiments, Compound 2 is administered in a dose of less than 200 mg. In some embodiments, Compound 2 is administered in a dose of between 100 mg and 200 mg. In some embodiments, Compound 2 is administered in a dose of less than 100 mg. In some embodiments, Compound 2 is administered in a dose of 150 mg twice a day.

In some embodiments, the administration regimen of the present disclosure comprises the oral administration of Compound 3, wherein Compound 3 is administered in a dosing regimen resulting in an AUC₍₀₋₂₄₎ of between about 400 ng*hr/ml and 4,000 ng*hr/ml. In some embodiments, Compound 3 is administered in a dosing regimen resulting in an AUC₍₀₋₂₄₎ of less than 2,500 ng*hr/mL. In some embodiments, Compound 3 is administered in a dosing regimen resulting in an AUC₍₀₋₂₄₎ of less than 1,500 ng*hr/mL. In other embodiments, the method is characterized by (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the retina of the eye compared to the plasma of the subject is maintained at no less than 0.60, no less than 0.65, no less than 0.70, or no less than 0.75 throughout the dosing period. In certain embodiments, the method is characterized by (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the choroid-retina pigmented epithelium of the eye compared to the plasma is maintained at no less than 2.5× throughout the dosing period. In some embodiments, the ratio of Compound 3 in the choroid-retina pigmented epithelium of the eye compared to the plasma is maintained between a range of 2.75 to 5.75 or greater throughout the dosing period. In particular embodiments, the method is characterized by (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the iris-ciliary body of the eye compared to the plasma of the subject is maintained at no less than 3 throughout the dosing period. In some embodiments, the ratio of Compound 3 in the iris-ciliary body compared to the plasma is maintained between a range of 3 to 5.5 or greater throughout the dosing period. In particular embodiments, the method includes orally administering Compound 3, or a pharmaceutically acceptable salt thereof, at a dosing regimen that provides a plasma AUC₀₋₂₄ of between about 400 ng*hr/ml and 4,000 ng*hr/ml, wherein the dosing regimen results in a depot of Compound 3 within an eye of the subject by day 15 of administration, and wherein the depot contributes to the effective amount of Compound 3 contained in the eye throughout the dosing period. In some embodiments, the dosing regimen results in a depot of Compound 3 within an eye of administration by day 8, by day 5, or by day 3 of administration. In some embodiments, Compound 3 is administered once a day in a dose of between about 50 mg and 500 mg. In some embodiments, Compound 3 is administered once a day. In some embodiments, Compound 3 is administered once a day in a dose of between about 20 mg and 350 mg. In some embodiments, Compound 3 is administered in a dose of less than 250 mg. In some embodiments, Compound 3 is administered in a dose of less than 150 mg. In some embodiments, Compound 3 is administered once a day in a dose of 150 mg.

The CFD inhibitor depot formed via oral or parenteral administration is capable of treating a complement-mediated disorder of the posterior, anterior, or other area of the eye.

In an embodiment of any of the above methods, the depot is formed in the eye of the subject without the use of any injections, implants or a medical device into the eye of the subject.

In an embodiment of any of the above methods, accumulation of the compound in an ocular tissue of the eye is tested, measured, or monitored by radiolabeling or positron emission tomography.

In another embodiment of any of the above methods, the method includes administering at least one additional active compound. The additional active compound can be an anti-VEGF compound. Alternatively, the additional active compound can be at least one complement 5 (C5) inhibitor.

In an embodiment of any of the above methods, the subject does not suffer from a melanin deficiency. Because of the effect ocular melanin plays in the advantageous pharmacokinetics of the compounds of Formulas I and II when systemically administered, the methods may not be suitable, e.g., for the treatment of subjects with acquired or hereditary disorders of pigmentation. Disorders of pigmentation can result from either an abnormal number of melanocytes, as in nevus of Ota and vitiligo, or an abnormal amount of melanin production, as in albinism. Melanin-producing cells are found in the skin, mucous membranes, uveal tract, and retinal pigment epithelium of the eye and the stria vascularis of the inner ear. Thus, many of the hereditary or congenital pigmentary disorders of the skin are associated with similar pigmentary abnormalities in the eye, such as iris heterochromia or changes in pigmentation of the fundus pigmentation deficiency disorders. Such disorders include those associated with hypomelanosis (i.e., loss of melanin) and hypopigmentation (e.g., oculocutaneous albinism or ocular albinism).

### BRIEF DESCRIPTION OF THE FIGURES

FIG. 1 is a cross-section of an eye that shows the location of all the distinct parts within the eye. Also included are magnified sections of the iris-ciliary body and the choroid-RPE and retina with additional detail.

FIG. 2 is a graph representing the concentration of [¹⁴C] Compound 1 in certain organ systems in a rat having undergone whole body autoradiography. The y-axis represents the concentration of [¹⁴C] Compound 1 measure in μg/equiv/g. The x-axis represents time from first administration of [¹⁴C] Compound 1 measured in hours.

FIG. 3 is a graph representing the concentration of [¹⁴C] Compound 1 in certain organ systems in a rat having undergone whole body autoradiography. The y-axis represents the concentration of [¹⁴C] Compound 1 measured in μg/equiv/g. The x-axis represents time from first administration of [¹⁴C] Compound 1 measured in hours.

FIG. 4 is a graph representing the concentration of [¹⁴C] Compound 2 in certain organ systems in a rat having undergone whole body autoradiography. The y-axis represents the concentration of [¹⁴C] Compound 2 measured in μg/equiv/g. The x-axis represents time from first administration of [¹⁴C] Compound 2 measured in hours.

FIG. 5 is a graph representing the concentration of Compound 1 after Day 1 in plasma and ocular tissues in New Zealand White rabbits after receiving a single 15 mg/kg dose of Compound 1. The y-axis represents the concentration of Compound 1 measured in ng/g. The x-axis represents time points following sample collection measured in hours.

FIG. 6 is a graph representing the concentration of Compound 1 after Day 1 in plasma and ocular tissues in Dutch Belted rabbits after receiving a single 15 mg/kg dose of Compound 1. The y-axis represents the concentration of Compound 1 measured in ng/g. The x-axis represents time points following sample collection measured in hours.

FIG. 7 is a graph representing the concentration of Compound 1 after Day 15 in plasma and ocular tissues in Dutch Belted rabbits after receiving a single 15 mg/kg dose of Compound 1. The y-axis represents the concentration of Compound 1 measured in ng/g. The x-axis represents time points following sample collection measured in hours.

FIG. 8 is a graph representing the plasma concentration of Compound 1 after Day 1 and Day 15 in New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 9 is a graph representing the mean retina concentration of Compound 1 after Day 1 and Day 15 in ocular tissues of New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 10 is a graph representing the mean Choroid-RPE concentration of Compound 1 after Day 1 and Day 15 in ocular tissues of New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 11 is a graph representing the mean iris-ciliary body concentration of Compound 1 after Day 1 and Day 15 in ocular tissues of New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 12 is a graph representing the mean Aqueous Humor concentration of Compound 1 after Day 1 and Day 15 in ocular tissues of New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 13 is a graph representing the mean Vitreous Humor concentration of Compound 1 after Day 1 and Day 15 in ocular tissues of New Zealand White and Dutch Belted rabbits after receiving a single 15 mg/kg dose of Compound 1 and 15 mg/kg BID Compound 1. The y-axis represents the concentration of Compound 1 measured in ng/g. The x-axis represents time points following sample collection measured in hours.

FIG. 14 is a graph representing the ocular tissue concentration of Compound 1 after Day 15 in New Zealand White and Dutch Belted rabbits after receiving 50 mg/kg BID Compound 1 for 15 days. The y-axis represents the concentration of Compound 1 measured in ng/g. The x-axis represents time points following sample collection measured in hours.

FIG. 15 is a graph representing the plasma concentration of Compound 1 after Day 1 and Day 15 in New Zealand White and Dutch Belted rabbits after receiving a single 15 mg/kg dose, 15 mg/kg BID for 15 days, and 50 mg/kg for 15 days. The y-axis represents the concentration of Compound 1 measured in ng/g. The x-axis represents time points following sample collection measured in hours.

FIGS. 16a and 16b are graphs representing the retina concentration of Compound 1 after Day 1 and Day 15 in New Zealand White and Dutch Belted rabbits after receiving a single 15 mg/kg dose, 15 mg/kg BID for 15 days, and 50 mg/kg for 15 days. The y-axis represents the concentration of Compound 1 measured in ng/g. The x-axis represents time points following sample collection measured in hours.

FIG. 17 is a graph representing the choroid-RPE concentration of Compound 1 after Day 1 and Day 15 in New Zealand White and Dutch Belted rabbits after receiving a single 15 mg/kg dose, 15 mg/kg BID for 15 days, and 50 mg/kg for 15 days. The y-axis represents the concentration of Compound 1 measured in ng/g. The x-axis represents time points following sample collection measured in hours.

FIG. 18 is a graph representing the iris-ciliary body concentration of Compound 1 after Day 1 and Day 15 in New Zealand White and Dutch Belted rabbits after receiving a single 15 mg/kg dose, 15 mg/kg BID for 15 days, and 50 mg/kg for 15 days. The y-axis represents the concentration of Compound 1 measured in ng/g. The x-axis represents time points following sample collection measured in hours.

FIG. 19 is a graph representing the plasma concentration of Compound 1 in healthy volunteers dosed orally at 200 mg, 600 mg, and 1200 mg. The y-axis represents the concentration of Compound 1 measured in ng/mL. The x-axis represents time points following sample collection measured in hours.

FIG. 20 is a graph representing the plasma concentration of Compound 1 in Dutch Belted rabbits after receiving a single 7.5 mg/kg dose; Dutch Belted rabbits after receiving 7.5 mg/kg BID for 15 days or 15 mg/kg BID for 15 days; and New Zealand White rabbits after receiving 15 mg/kg BID for 15 days. The y-axis represents the concentration of Compound 1 measured in ng/mL. The x-axis represents time points following sample collection measured in hours.

FIG. 21 is a graph representing the choroid-RPE concentration of Compound 1 in Dutch Belted rabbits after receiving a single 7.5 mg/kg dose; Dutch Belted rabbits after receiving 7.5 mg/kg BID for 15 days or 15 mg/kg BID for 15 days; and New Zealand White rabbits after receiving 15 mg/kg BID for 15 days. The y-axis represents the concentration of Compound 1 measured in ng/g. The x-axis represents time points following sample collection measured in hours.

FIG. 22 is a graph representing the iris-ciliary body concentration of Compound 1 in Dutch Belted rabbits after receiving a single 7.5 mg/kg dose; Dutch Belted rabbits after receiving 7.5 mg/kg BID for 15 days or 15 mg/kg BID for 15 days; and New Zealand White rabbits after receiving 15 mg/kg BID for 15 days. The y-axis represents the concentration of Compound 1 measured in ng/g. The x-axis represents time points following sample collection measured in hours.

FIG. 23 is a graph representing the retina concentration of Compound 1 in Dutch Belted rabbits after receiving a single 7.5 mg/kg dose; Dutch Belted rabbits after receiving 7.5 mg/kg BID for 15 days or 15 mg/kg BID for 15 days; and New Zealand White rabbits after receiving 15 mg/kg BID for 15 days. The y-axis represents the concentration of Compound 1 measured in ng/g. The x-axis represents time points following sample collection measured in hours.

FIG. 24 is a graph representing Compound 1 binding to synthetic melanin. The y-axis represents the concentration of bound Compound 1 measured in μM. The x-axis represents the concentration of free Compound 1 measured in μM.

FIG. 25 is a graph representing Compound 1 binding to Sepia officinalis melanin. The y-axis represents the concentration of bound Compound 1 measured in μM. The x-axis represents the concentration of free Compound 1 measured in μM.

FIG. 26 is a graph representing chloroquine binding to synthetic melanin. The y-axis represents the concentration of bound chloroquine measured in μM. The x-axis represents the concentration of free chloroquine measured in μM.

FIG. 27 is a graph representing chloroquine binding to Sepia officinalis melanin. The y-axis represents the concentration of bound chloroquine measured in μM. The x-axis represents the concentration of free chloroquine measured in μM.

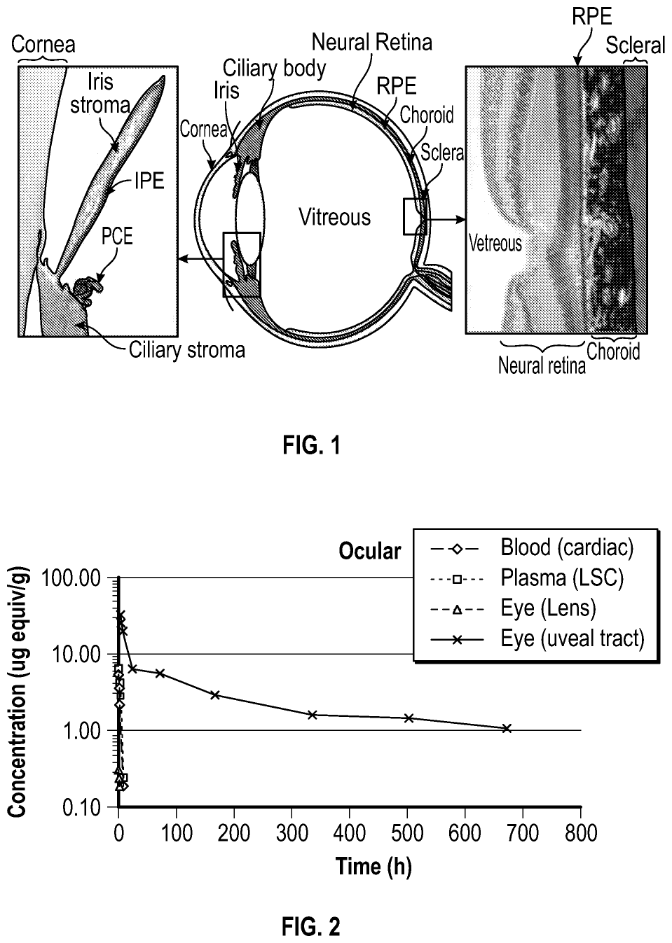

### DETAILED DESCRIPTION

Selected complement factor D inhibitor compounds accumulate disproportionately in ocular tissue over plasma and other tissues on systemic administration, which unexpectedly results in a depot of the compound in the choroid-retina pigmented epithelium (C-RPE) and/or the iris-ciliary body (I-CB), without the required use of an injection or medical device. Based on this, a method to treat an ocular disorder mediated by complement factor D is provided, wherein a compound depot of the identified complement factor D inhibitor is created and provided based on systemic (i.e., oral or parenteral) delivery to the patient, for example a human, in need thereof, wherein the compound accumulates in the choroid-retina pigmented epithelium (C-RPE) and/or the iris-ciliary body (I-CB) of the eye and is available to provide extended therapy after cessation of the systemic delivery of the compound. In another embodiment, a method is provided that includes providing a systemic dosage that is less then what would be administered for a systemic complement factor D mediated disorder (for example PNH, which is Paroxysmal Nocturnal Hemoglobinuria, C₃G, or aHUS) but which is sufficient to form an accumulation in the choroid-retina pigmented epithelium (C-RPE) and/or the iris-ciliary body (I-CB) of the eye over time, which results in an advantageous ocular therapy with less toxicity.

The extended ocular release of the complement factor D compound can be confirmed by its therapeutic effect over time against the complement factor D ocular disorder being treated, after cessation of systemic treatment. The depot can also be confirmed using compound radiolabeling, as well known in the art and described below.

### Definitions

Compounds are described using standard nomenclature. Unless defined otherwise, all technical and scientific terms used herein have the same meaning as is commonly understood by one of skill in the art to which this disclosure belongs.

“Complement-mediated disorder” as used herein means a disorder mediated by dysfunction of or excessive activation of complement.

“Cₘₐₓ” (Amount/volume) as used herein means the maximum (peak) plasma drug concentration.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the disclosure, and does not pose a limitation on the scope of the invention which is only defined by the claims. Unless defined otherwise, technical and scientific terms used herein have the same meaning as is commonly understood by one of skill in the art to which this disclosure belongs.

The compounds of the present disclosure may form a solvate with solvents (including water). Therefore, one embodiment includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone and other common organic solvents. The term “hydrate” refers to a molecular complex comprising a compound of the disclosure and water. Pharmaceutically acceptable solvates in accordance with the disclosure include those wherein the solvent of crystallization may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

An “ocular disease” herein refers to any condition, disease, or disorder that interferes with the ability for the eye to function properly and/or negatively affects the visual clarity of the eye. Herein, ocular is used synonymously with ophthalmic, ophthalmology or eye. Herein, ocular disease covers all areas of disease in ophthalmology for the comprehensive information you need for managing clinical cases; and any ocular manifestation of a systemic disease (disease known to cause ocular or visual changes). Examples of ocular diseases include, but are not limited to, age-related macular degeneration (AMD), geographic atrophy, and diabetic retinopathy.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

An “administration” herein refers to a means of providing the compounds described herein to a subject by a suitable method.

“Pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable salt” is a derivative of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Thus, the term “salt” refers to the relatively non-toxic, inorganic, and organic acid addition salts of compounds of the presently disclosed subject matter. These salts can be prepared in situ during the final isolation and purification of the compounds or by separately reacting the purified Compound 1 in its free base form with a suitable organic or inorganic acid and isolating the salt thus formed. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

The term “carrier” applied to pharmaceutical compositions/combinations of the disclosure refers to a diluent, excipient, or vehicle with which an active compound is provided.

A “pharmaceutically acceptable excipient” means an excipient that is useful in preparing a pharmaceutical composition/combination that is generally safe, non-toxic, and neither biologically nor otherwise inappropriate for administration to a subject, typically a human. In one embodiment, an excipient is used that is acceptable for veterinary use. In one embodiment, an excipient is used that is acceptable for mammalian, particularly human, use.

The “subject” treated is typically a human subject, although it is to be understood the methods described herein are effective with respect to other animals, such as mammals and vertebrate species. More particularly, the term “subject” can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like. In a preferred embodiment, the subject is a human.

The term “providing a compound with at least one additional active agent,” for example, in one embodiment can mean that the compound and the additional active agent(s) are provided simultaneously in a single dosage form, provided concomitantly in separate dosage forms, or provided in separate dosage forms for administration. In one embodiment, the compound administrations are separated by some amount of time that is within the time in which both the compound and the at least one additional active agent are within the blood stream of a patient. In certain embodiments, the compound and the additional active agent need not be prescribed for a patient by the same medical care worker. In certain embodiments, the additional active agent or agents need not require a prescription. Administration of the compound or the at least one additional active agent can occur via any appropriate route, for example, oral tablets, oral capsules, oral liquids, inhalation, injection, suppositories, parenteral, sublingual, buccal, intravenous, intraaortal, transdermal, polymeric controlled delivery, non-polymeric controlled delivery, nano or microparticles, liposomes, and/or topical contact.

As used herein, the terms “active agent,” or “active compound,” means one or more of the presently described chemical compounds that are useful to treat any of the disorders described herein, or to control or improve the underlying cause or symptoms associated with any physiological or pathological disorder described herein in a subject, typically a human. Particular examples used herein are the compounds of Formula I, the compounds of Formula II, Compound 1, Compound 2, and Compound 3.

As used herein, the term “prodrug” means a compound which when administered to a subject in vivo is converted into the compound (the “parent compound”). Prodrugs can be used to achieve any desired effect, including to enhance properties of the parent compound or to improve the pharmaceutic or pharmacokinetic properties of the parent compound. Prodrug strategies exist which provide choices in modulating the conditions for in vivo generation of the compound, all of which are deemed included herein. Non-limiting examples of prodrug strategies include covalent attachment of removable groups, or removable portions of groups, for example, including but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride derivatives, among others.

A “therapeutically effective amount” means an amount of at least one active agent which is effective, when administered to a subject, to provide a therapeutic benefit such as an amelioration of symptoms, slowing the progression of disease, or reduction or elimination of the disease itself.

“Prevent,” “preventing,” or “prevention” as used herein refers to a means of avoiding or delaying the onset of symptoms associated with a disease or condition in a subject that has not developed such symptoms at the time of administering a compound or composition.

“Treating”, “treatment,” or “treat” herein refers to a means of reducing the frequency or severity with which symptoms of a disease or condition are experienced by a subject, or slowing the progression of disease in a subject by administering a compound or composition to the subject.

Throughout the specification and claims, a given chemical formula or name shall encompass all optical and stereoisomers, as well as racemic mixtures where such isomers and mixtures exist, unless otherwise noted.

In one embodiment, the compounds of Formula I or Formula II include desired isotopic substitutions of atoms, at amounts above the natural abundance of the isotope, i.e., enriched. Isotopes are atoms having the same atomic number but different mass numbers, i.e., the same number of protons but a different number of neutrons. By way of general example, and without limitation, isotopes of hydrogen, for example, deuterium (²H) and tritium (³H) may be used anywhere in described structures. Alternatively, or in addition, isotopes of carbon, e.g., ¹³C and u may be used. In some embodiments, an isotope of ¹⁸F may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of, or eliminate, the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby a H-isotope, i.e., hydrogen/protium (¹H), is substituted by a H-isotope, i.e., deuterium (²H). Deuterium substitution can be partial or complete. Partial deuterium substitution means that at least one hydrogen is substituted by at least one deuterium. In certain embodiments, the isotope is 90%, 95%, or 99% or more enriched in an isotope at any location of interest. In some embodiments, it is deuterium that is 90%, 95%, or 99% enriched at a desired location.

In the description herein generally, whenever any of the terms referring to the compounds of Formula I, the compounds of Formula II, Compound 1, Compound 2, and Compound 3 are used, it should be understood that pharmaceutically acceptable salts, prodrugs, or compositions are considered included, unless otherwise stated or inconsistent with the text.

As contemplated herein and for purposes of the disclosed ranges herein, all ranges described herein include any and all numerical values occurring within the identified ranges. For example, a range of 1 to 10, or between 1 and 10, as contemplated herein, would include the numerical values 1, 2, 3, 4, 5, 6, 7, 8, 9, 10, as well as fractions thereof.

Complement Factor D Inhibitors

As provided herein, the complement factor D (CFD) inhibitors for use in the present disclosure are selected from compounds of Formula I and Formula II or pharmaceutically acceptable salts, N-oxides, isotopic derivatives, or prodrugs thereof. Formula I and Formula II are provided below:

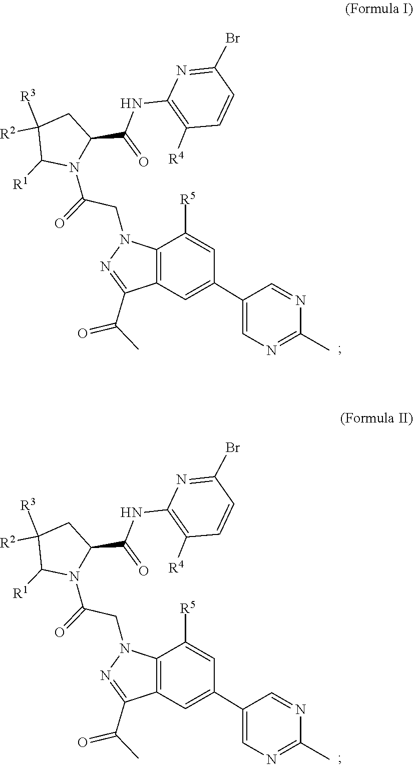

wherein:
R¹ and R² are:
i. both hydrogen; or
ii. combined together to form a cyclopropyl ring;
R³ is hydrogen, methyl, or fluoro;
R⁴ is hydrogen or C₁-C₄ alkyl; and
R⁵ is hydrogen or methyl;
   or a pharmaceutically acceptable salt, an N-oxide, isotopic derivative, or prodrug thereof.

In some embodiments, the compound of Formula I is selected from Compound 1 and Compound 2, wherein Compound 1 and Compound 2 have the structure:

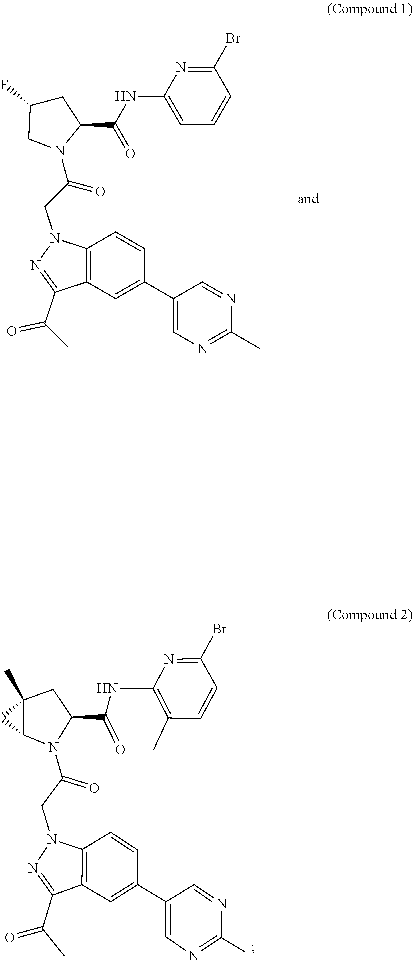 [SMILES: CC(=O)c1nn(CC(=O)N2C[C@H](F)C[C@H]2C(=O)Nc2cccc(Br)n2)c2ccc(-c3cnc(C)nc3)cc12 ; CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, the compound of Formula II is Compound 3 having the structure:

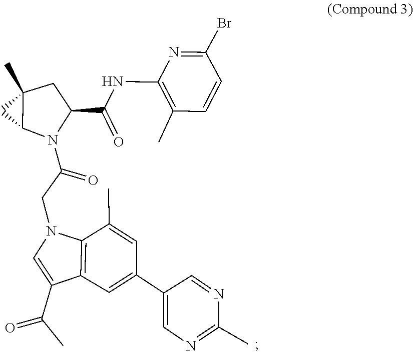 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

An exemplary CFD inhibitor for use in the present disclosure is, for example, Compound 1. Compound 1 is a potent Factor D inhibitor, having an inhibitory effect on CFD of IC50=0.54 nM, and an inhibition of catalytic activity of CFD against Factor B of IC₅₀=25 nM. Compound 1 also strongly inhibits AP activity in vitro, showing an IC₅₀ of 27 nM for rabbit erythrocyte hemolysis, 14 nM for PNH erythrocyte hemolysis, and 26 nM by Wieslab assay.

An exemplary CFD inhibitor for use in the present disclosure is, for example, Compound 2. Compound 2 is a potent Factor D inhibitor, having an inhibitory effect on CFD of IC₅₀=26.8 nM.

An exemplary CFD inhibitor for use in the present disclosure is, for example, Compound 3. Compound 3 is a potent Factor D inhibitor, having an inhibitory effect on CFD of IC₅₀=32.1 nM.

An exemplary method of making Compound 1 is provided below:

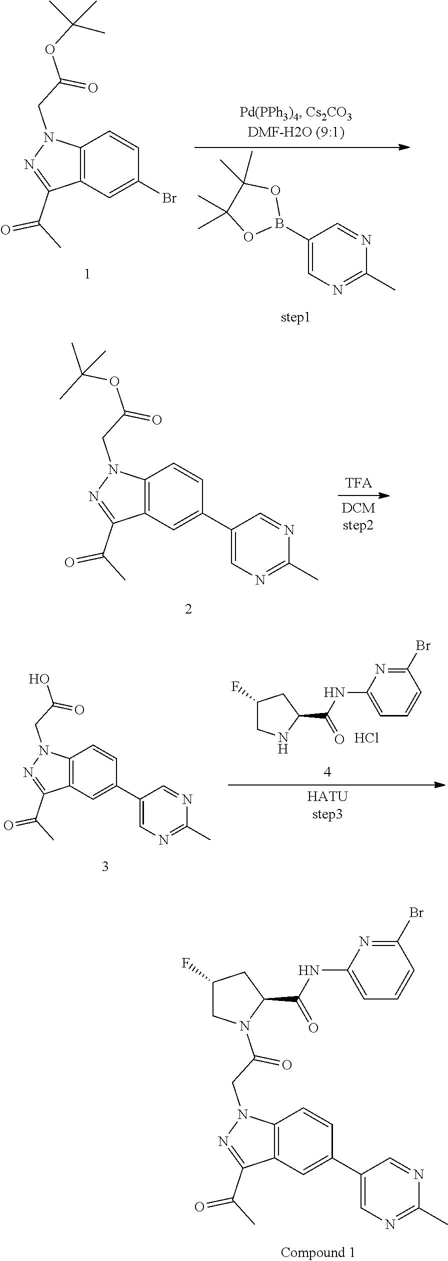

Compounds of Formula I can be synthesized using the methods disclosed in US 2015/0239895 and US 2017/0066783, each of which is incorporated herein by reference in its entirety. Complement factor D inhibitor ((2S,4R)-1-(2-(3-Acetyl-5-(2-methylpyrimidin-5-yl)-1H-indazol-1-yl)acetyl)-N-(6-bromopyridin-2-yl)-4-fluoropyrrolidine-2-carboxamide) (Compound 1) has been previously described, see US 2015/0239895 and US 2017/0066783. Compound 1 may be synthesized by methods known to those in the art.

In step 1, tert-butyl 2-(3-acetyl-5-bromo-1H-indazol-1-yl)acetate (intermediate 1) is coupled to 2-methyl-5-(4,4,5,5-tetramethyl-1,3,2-dioxaborolan-2-yl)pyrimidine using tetrakis(triphenylphosphine)palladium(0) in the presence of a base to provide tert-butyl 2-(3-acetyl-5-(2-methylpyrimidin-5-yl)-1H-indazol-1-yl)acetate (intermediate 2).

In step 2, hydrolysis of tert-butyl 2-(3-acetyl-5-(2-methylpyrimidin-5-yl)-1H-indazol-1-yl)acetate (intermediate 2) with trifluoroacetic acid provides 2-(3-acetyl-5-(2-methylpyrimidin-5-yl)-1H-indazol-1-yl)acetic acid (intermediate 3).

In step 3, 2-(3-acetyl-5-(2-methylpyrimidin-5-yl)-1H-indazol-1-yl)acetic acid (S3) and (2S,4R)-N-(6-bromopyridin-2-yl)-4-fluoropyrrolidine-2-carboxamide (compound 4) are coupled using HATU to provide (2S,4R)-1-(2-(3-Acetyl-5-(2-methylpyrimidin-5-yl)-1H-indazol-1-yl)acetyl)-N-(6-bromopyridin-2-yl)-4-fluoropyrrolidine-2-carboxamide (Compound 1).

An exemplary method of making Compound 2 is provided below:

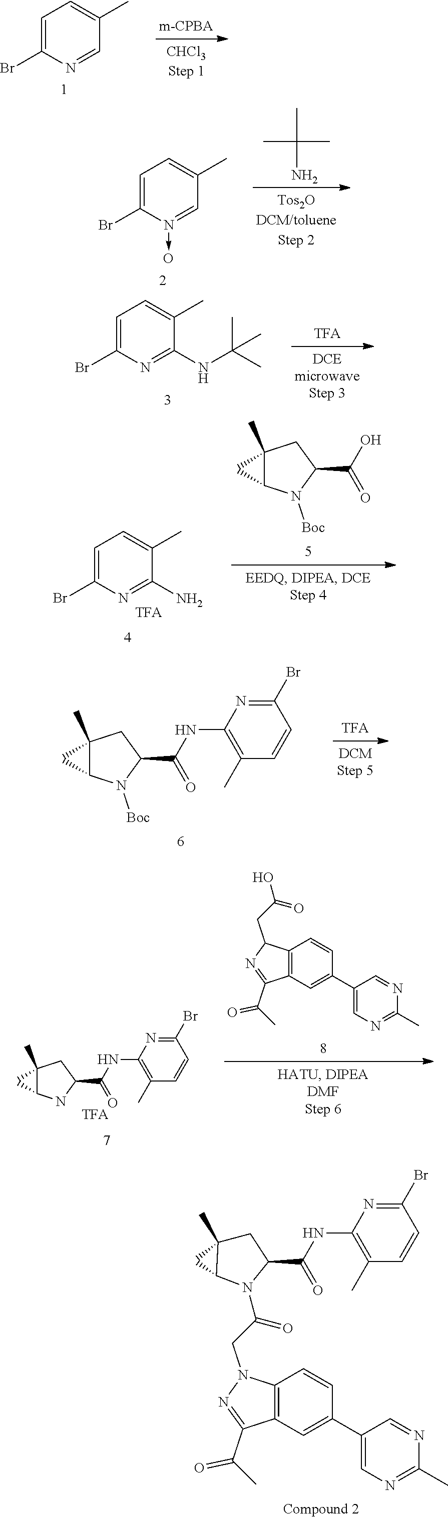

Compounds of Formula I can be synthesized using the method disclosed in WO 2018/160889, incorporated herein by reference in its entirety. Complement factor D inhibitor ((1R,3S,5R)-2-(2-(3-acetyl-5-(2-methylpyrimidin-5-yl)-1H-indazol-1-yl)acetyl)-N-(6-bromo-3-methylpyridin-2-yl)-5-methyl-2-azabicyclo[3.1.0]hexane-3-carboxamide) (Compound 2) has been previously described, see WO 2018/160889. Compound 2 may be synthesized by methods known to those in the art.

In step 1, a mixture of 2-bromo-5-methylpyridine (4.0 g, 23.3 mmol) in CHCl₃ (20 mL) was added m-CPBA (5.2 g, 29.8 mmol) and the reaction was stirred at 50° C. for 3 hrs. The mixture was cooled and filtered. The filtrate was washed with 5% aqueous NaOH solution and the organic layer was separated, dried over anhydrous Na₂SO₄ and concentrated to dryness. The residue was purified by column chromatography on silica gel (eluted with DCM:MeOH=100:0 to 100:1) to give the intermediate 2 (3.1 g, 71% yield) as a colorless oil. LC/MS (ESI) m/z: 188 (M+H)⁺.

In step 2, a solution of intermediate 2 (1.18 g, 6.28 mmol) in toluene (22 mL) was added 2-methylpropan-2-amine (3.21 g, 44 mmol) and a solution of 4-methylbenzenesulfonic anhydride (6.76 g, 20.74 mmol) in DCM (48 mL) dropwise at 0° C. The reaction mixture was stirred at 0° C. for 2 hrs. and filtered. The filtrate was concentrated under reduced pressure to give a residue, which was purified by column chromatography on silica gel (eluted with PE:EtOAc=30:1) to give the intermediate 3 (280 mg, 18% yield) as a white solid. LC/MS (ESI) m/z: 243 (M+H)⁺.

In step 3, a mixture of intermediate 3 (280 mg, 1.15 mmol) in DCE (1 mL) was added TFA (1 mL). The reaction was heated to 90° C. for 25 min in a microwave reactor. The mixture was cooled and concentrated under reduced pressure. The residue was re-crystallized with Et₂O/PE to give the intermediate 4 (340 mg, 86% yield) as white solid. LC/MS (ESI) m/z: 187 (M+H)⁺.

In step 4, a solution of intermediate 4 (186 mg, 0.77 mmol), compound 5 (230 mg, 0.77 mmol) and EEDQ (384 mg, 1.54 mmol) in DCE (20 mL) was added DIPEA (410 mg, 3.08 mmol). The reaction was stirred at reflux overnight under N₂ atmosphere. The mixture was concentrated and the residue was purified by column chromatography on silica gel (eluted with petroleum ether:ethyl acetate=10:1 to 3:1) to give the intermediate 6 (220 mg, 72.7% yield) as a yellow solid. LC/MS (ESI) m/z: 410 (M+H)⁺.

In step 5, a mixture of intermediate 6 (220 mg, 0.56 mmmol) in DCM (2 mL) was added TFA (2 mL) and the reaction was stirred at room temperature for 2 hrs. The mixture was concentrated to dryness to give the intermediate 7 (220 mg, 100 yield) as a brown solid, which was directly used to the next reaction without further purification. LC/MS (ESI) m/z: 310 (M+H)⁺.

In step 6, a solution of intermediate 7 (168 mg, 0.54 mmol), compound 8 (220 mg, 0.54 mmol) and DIPEA (210 mg, 1.62 mmol) in DMF (10 mL) was added HATU (412 mg, 1.08 mmol) at 0° C. The reaction was stirred at room temperature overnight. The mixture was diluted with EtOAc, washed with 10% aq. LiCl solution and brine successively, dried over anhydrous Na₂SO₄, filtered and then concentrated. The residue was purified by prep-HPLC (eluted with CH₃CN/water) to give afford ((1R,3S,5R)-2-(2-(3-acetyl-5-(2-methylpyrimidin-5-yl)-1H-indazol-1-yl)acetyl)-N-(6-bromo-3-methylpyridin-2-yl)-5-methyl-2-azabicyclo[3.1.0]hexane-3-carboxamide) Compound 2 (71 mg, 21.8% yield) as a white solid. ¹H NMR (400 MHz, DMSO-d6) δ: 10.29 (s, 1H), 9.05 (s, 2H), 8.45 (s, 1H), 7.86 (d, J 1.6 Hz, 2H), 7.63 (d, J 8.0 Hz, 1H), 7.45 (d, J 7.9 Hz, 1H), 5.93 (d, J=17.3 Hz, 1H), 5.58 (d, J=17.2 Hz, 1H), 4.41 (dd, J=9.3, 5.1 Hz, 1H), 3.60 (dd, J=5.4, 2.4 Hz, 1H), 2.69 (s, 3H), 2.65 (s, 3H), 2.53-2.59 (m, 1H), 2.00-2.09 (m, 4H), 1.33 (s, 3H), 0.98-1.07 (m, 2H). LC/MS (ESI) m/z: 602 (M+H)⁺.

An exemplary method of making Compound 3 is provided as follows:

Complement factor D inhibitor ((1R,3S,5R)-2-(2-(3-acetyl-7-methyl-5-(2-methylpyrimidin-5-yl)-1H-indol-1-yl)acetyl)-N-(6-bromo-3-methylpyridin-2-yl)-5-methyl-2-azabicyclo[3.1.0]hexane-3-carboxamide) (Compound 3) has been previously described, See US2020/0071301A1. Compound 3 may be synthesized by methods known to those in the art. (1R,3S,5R)-2-(2-(3-Acetyl-7-methyl-5-(2-methylpyrimidin-5-yl)-1H-indol-1-yl)acetyl)-N-(6-bromo-3-methylpyridin-2-yl)-5-methyl-2-azabicyclo[3.1.0]hexane-3-carboxamide Compound 3 ¹H NMR (400 MHz, Chloroform-d) δ 0.87 (dd, J=2.4, 5.4 Hz, 1H), 1.15 (t, J=5.4 Hz, 1H), 1.42 (s, 3H), 2.08 (s, 3H), 2.30-2.36 (m, 1H), 2.53 (s, 3H), 2.67 (d, J=14. 8 Hz, 1H), 2.72 (s, 3H), 2.79 (s, 3H), 3.10 (d, J=2.8, 1H), 4.83 (d, J=8.3 Hz, 1H), 5.30 (d, J=17.7 Hz, 1H), 5.46 (d, J=17.7 Hz, 1H), 7.16-7.26 (m, 2H), 7.34 (d, J=7.9 Hz, 1H), 7.71 (s, 1H), 8.55 (s, 1H), 8.56 (brs, 1H), 8.89 (s, 2H).

A general scheme for the synthesis of a compound of Formula I or Formula II, for example, Compound 3 is shown below, from a central core of any one of Routes 1-5; and a compound of Formula II, for example Compound 3, can be prepared as shown below, in Route 6 and 7, incorporated herein.

In one embodiment, a compound of Formula I or II, for example Compound 3, can be prepared wherein the central core Structure 1 is an N-protected amino acid where X¹ is nitrogen and PG is a protecting group. In one embodiment, the central core of Structure 1 is coupled to an amine to generate an amide of Structure 2 (wherein L-B includes a C(O)N moiety). Structure 2 can then be deprotected to generate Structure 3. Structure 3 is coupled to Structure 4 (A-COOH) to generate a second amide bond, forming a compound of Formula I or Formula II, for example, Compound 3. The chemistry is illustrated in Route 1.

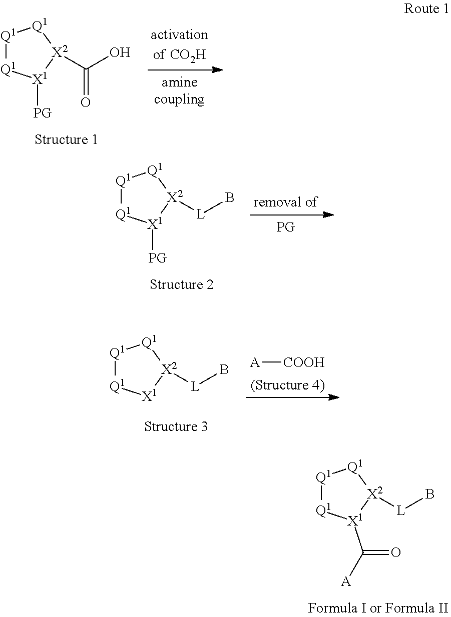

In an alternative embodiment, a compound of Formula I or II, for example Compound 3, can be prepared, wherein the central core Structure 5 is reacted with a heterocyclic or heteroaryl compound to generate a compound of Structure 6. In one embodiment, Structure 6 is deprotected to generate a carboxylic acid, Structure 7. In one embodiment, Structure 7 is coupled to an amine to generate a compound of Formula I or II, for example, Compound 3. This chemistry is illustrated in Route 2.

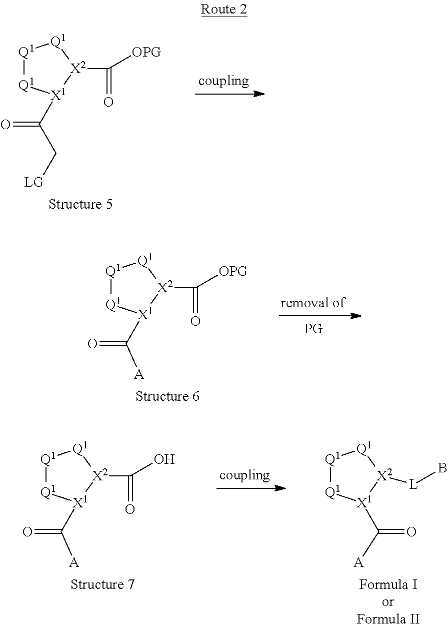

In an alternative embodiment, a compound of Formula I or II, for example Compound 3, can be prepared, wherein the central core Structure 8 is deprotected to generate an amine which is Structure 9. Structure 9 is then coupled to generate an amide which is Structure 6. Structure 6 is then deprotected to generate a carboxylic acid which is Structure 7. Structure 7 is then coupled to form the amide which falls within a compound of Formula I or Formula II, for example Compound 3. The chemistry is illustrated in Route 3.

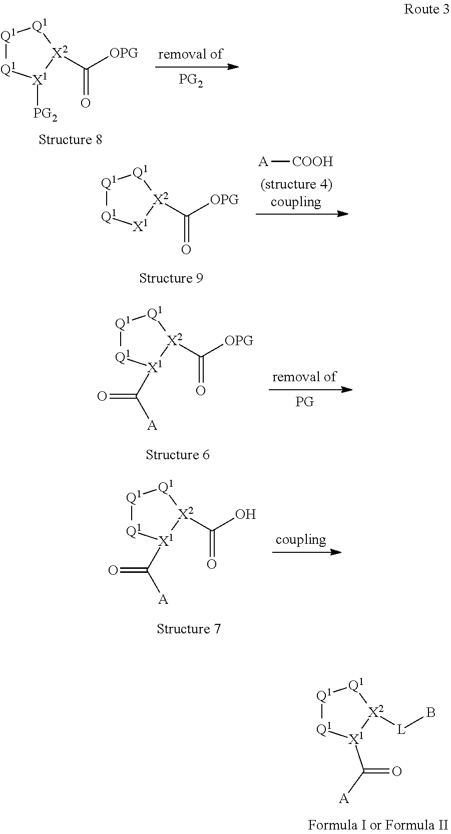

In an alternate embodiment, a compound of Formula I or Formula II, for example, Compound 3 can be prepared, wherein a heteroaryl or aryl moiety 4-1 is coupled to a central core to generate 4-2. The protected acid, 4-2 is deblocked to form the carboxylic acid, 4-3. The carboxylic acid is then coupled to form an amide (L-B) which is 4-4. The heteroaryl or aryl moiety, A′, can then be further derivatized to add substituents at the X¹¹, X¹², X¹³ and X¹⁴ positions to generate a compound of Formula I or Formula II, for example, Compound 3. This chemistry is illustrated in Route 4.

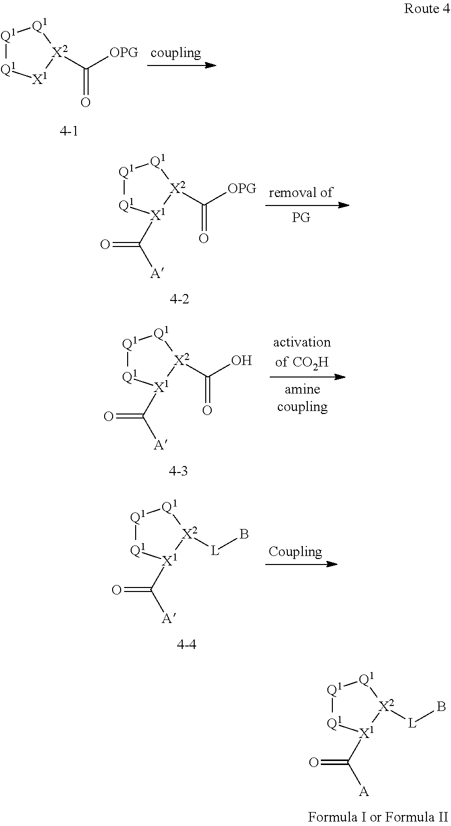

In an alternate embodiment, a compound of Formula I or Formula II, for example, Compound 3 can be prepared, wherein the central core Structure 5-1 is coupled to an acid, Structure 5-2, to generate Structure 5-3. The carboxylic acid, Structure 5-3, is deblocked to generate a carboxylic acid which is Structure 5-4. Carboxylic acid Structure 5-4 is coupled to an amine to form the product amide (L-B), a compound of Formula I or Formula II, for example, Compound 3. This chemistry is illustrated in Route 5.

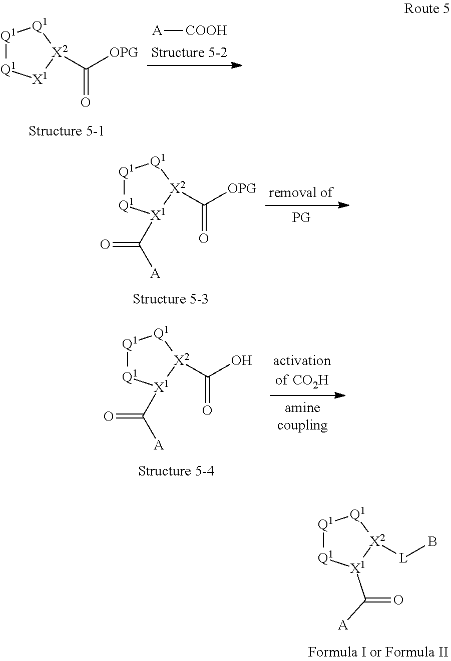

In an alternate embodiment, a compound of Formula II, for example, Compound 3 can be prepared, wherein a heteroaryl compound of Structure 10 is acylated to generate a compound of Structure 11, wherein LG is a leaving group. As an example, the leaving group can be a halide, for example bromide. Structure 11 is coupled to Structure 12 to generate Structure 13. In some embodiments, LG₁ is a leaving group. In some embodiments, the LG₁ is a halide. Structure 13 is coupled to an aryl, heteroaryl or heterocylic compound to generate Structure 14. In some embodiments, Structure 13 is treated with an aryl, heteroaryl or heterocylic boronic acid, an organometallic catalyst, a base and an organic solvent. In some embodiments, the organometallic catalyst is tetrakis(triphenylphosphine)palladium (0). In some embodiments, the base is cesium carbonate. In some embodiments, the organic solvent is DMF. Structure 14 is treated with an organic acid such as, but not limited to, trifluoroacetic acid, to generate Structure 15. Structure 15 is coupled to Structure 3 from Route 1 to generate a compound of Formula II, for example, Compound 3. This chemistry is illustrated in Route 6.

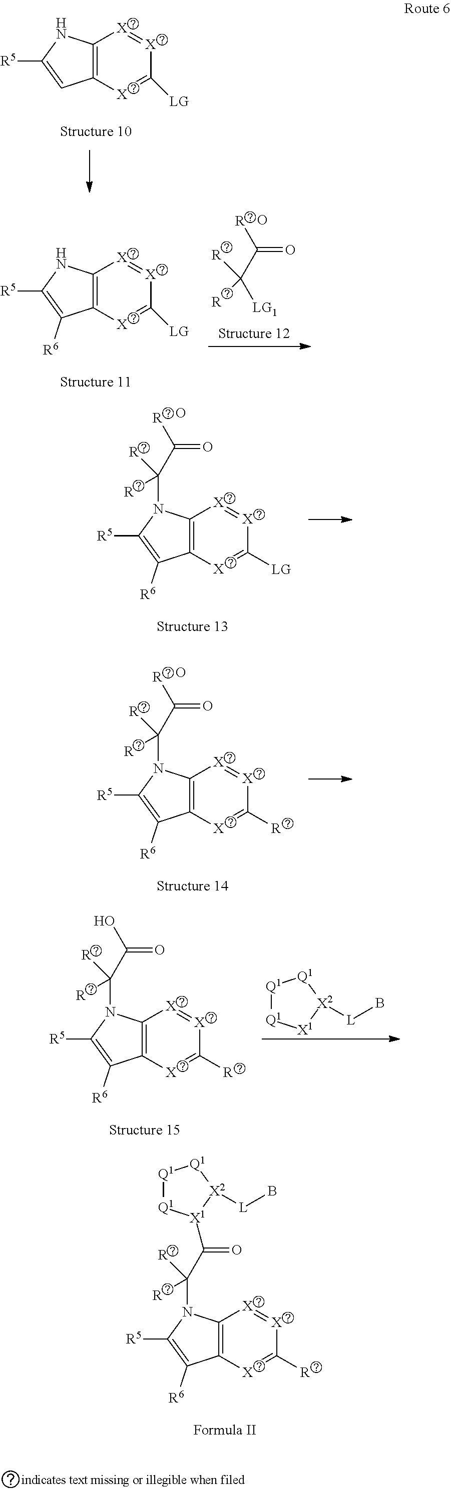

In an alternate embodiment, a compound of Formula II, for example, Compound 3 can be prepared, wherein a heteroaryl compound of Structure 17 is acylated to generate a compound of Structure 18, wherein LG is a leaving group. As an example, the leaving group can be a halide, for example bromide, Structure 18 is coupled to an activated ester, Structure 12 from Route 6, wherein LG₁ can be a halogen to generate Structure 19. Structure 19 is coupled to an aryl, heteroaryl or heterocylic compound to generate Structure 20. In some embodiments, Structure 19 is treated with an aryl, heteroaryl or heterocylic boronic acid, an organometallic catalyst, a base and an organic solvent. In some embodiments, the organometallic catalyst is tetrakis(triphenylphosphine)palladium (0). In some embodiments, the base is cesium carbonate. In some embodiments, the organic solvent is DMF. Structure 20 is treated with an organic acid such as, but not limited to, trifluoroacetic acid to generate Structure 21. Structure 21 is coupled to Structure 3 from Route 1 to generate a compound of Formula II, for example, Compound 3. This chemistry is illustrated in Route 7.

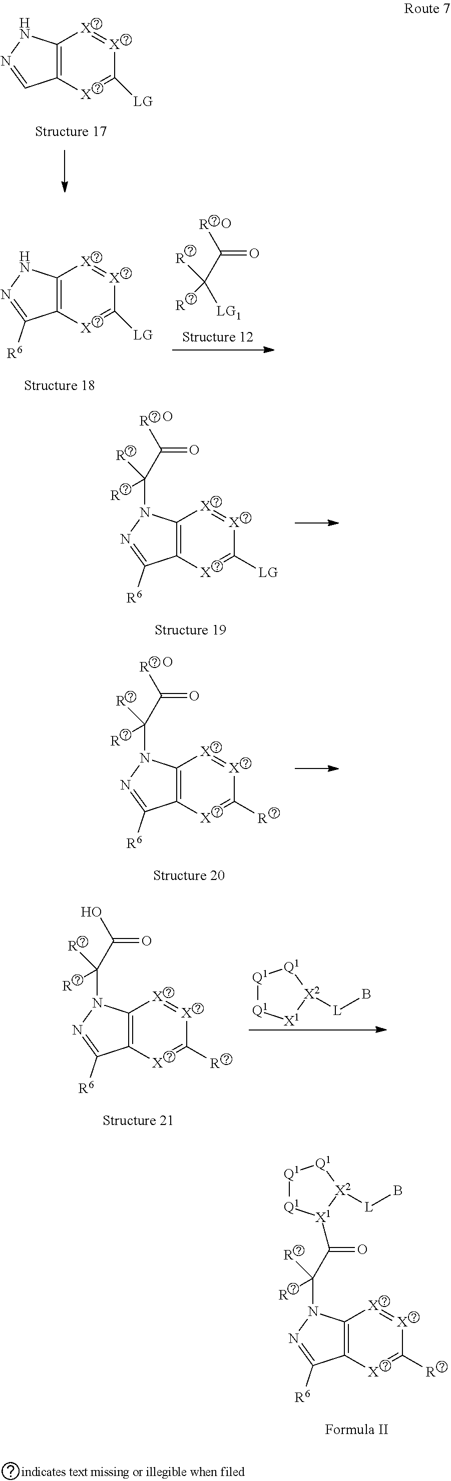

Complement-Mediated Ocular Disorders

The disclosure includes methods for treating a complement-mediated disorder of the eye caused by a dysfunction of or excessive activation of complement factor D. Complement-mediated disorders that affect the eyes are known. The CFD inhibitor depot formed via oral or parenteral administration as described herein is capable of treating a complement-mediated disorder of the posterior, anterior, or other area of the eye. Posterior ocular disorders that can be treated with an effective amount of one of the compounds described herein include, but are not limited to, dry and wet age-related macular degeneration (AMD), cytomegalovirus (CMV) infection, diabetic retinopathy, choroidal neovascularization, acute macular neuroretinopathy, macular edema, Behcet's disease, retinal disorders, diabetic retinopathy, retinal arterial occlusive disease, central retinal vein occlusion, uveitic retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction, and retinitis pigmentosa.

Anterior ocular disorders that can be treated with an effective amount of one of the compounds described herein include, but are not limited to, glaucoma, allergic conjunctivitis, anterior uveitis, and cataracts.

In particular embodiments, the disorder is selected from wet AMD, dry AMD, geographic atrophy (GA), autoimmune uveitis, and a diabetic retinopathy. Age-related macular degeneration (AMD) is a disease that impacts the central area of the retina in the eye, called the macula. AMD is a leading cause of blindness in people age 60 and over. An estimated 17 million people worldwide are affected by AMD.

There are two main types of AMD dry (non-neovascular) form and wet (neovascular) form. Dry form AMD occurs when parts of the macula get thinner with age and yellow deposits/clumps, called drusen, grow on the macula. These clumps can get bigger and more numerous resulting in distorted vision. As the condition progresses, the light-sensitive cells in the macula get thinner and eventually die. In the atrophic form, blind spots in the center of vision occur, resulting in a loss of central vision. Wet form AMD is less common but is more serious and occurs when new abnormal blood vessels grow underneath the macula. These blood vessels may leak blood and fluid into the retina, distorting vision so that straight lines look wavy. As the condition progresses these blood vessels and their bleeding eventually form scars, leading to permanent loss of central vision.

In some embodiments, the complement-mediated ocular disorders is geographic atrophy (GA). GA is a chronic progressive degeneration of the macula, and is considered as part of late-stage age-related macular degeneration (AMD). The condition leads to central scotomas and permanent loss of visual acuity. The disease is characterized by localized sharply demarcated atrophy of outer retinal tissue, retinal pigment epithelium and choriocapillaris. It starts typically in the perifoveal region and expands to involve the fovea with time, leading to central scotomas and permanent loss of visual acuity. It is bilateral in most cases. Over 8 million people are affected worldwide with GA, approximately 20% of all individuals with AMD.

In some embodiments, the complement-mediated ocular disorder is uveitis. Uveitis is an eye inflammation affecting the uvea, the area of the eye that includes the iris, choroid, and the ciliary body. Uveitis can also affect nearby parts of the eye, like the retina, vitreous, and optic nerve. Non-infectious uveitis can be acute or chronic in one or both eyes. There are several types of non-infectious uveitis such as intermediate uveitis, posterior uveitis and pan-uveitis, each correlating to the area of the eye that is affected. Symptoms include changes in vision, blurred vision, dark floating spots (floaters), sensitivity to light and/or eye pain or redness. In children, symptoms can also include eye redness, crossed eye, different size pupils, eyes look milky or cloudy and headaches or vision problems. A sudden appearance or worsening of symptoms is called a ‘flare’ which can recur over time.

In some embodiments, the complement-mediated disorder is diabetic retinopathy. Diabetic retinopathy is a complication of diabetes that causes damage to the blood vessels of the retina, the light-sensitive tissue that lines the back part of the eye. The elevated sugar levels in diabetic patients can damage the small blood vessels that nourish the retina, and may, in some cases, block these blood vessels completely. Diabetic retinopathy is the most common cause of irreversible blindness in working-age Americans, as many people with type 1 diabetes suffer blindness as well as those with the more common type 2 disease. Diabetic retinopathy occurs in more than half of the people who develop diabetes.

There are two types of diabetic retinopathy: early/non-proliferative and advanced/proliferative diabetic retinopathy. In early diabetic retinopathy, new blood vessels do not grow (proliferate), therefore, the walls of the blood vessels in the retina weaken. Tiny bulges (microaneurysms) protrude from the vessel walls of the smaller vessels, sometimes leaking fluid and blood into the retina. In advanced diabetic retinopathy, damaged blood vessels close off, causing the growth of new, abnormal blood vessels in the retina, and can leak into the clear, jelly-like substance that fills the center of the eye. Eventually, scar tissue stimulated by the growth of new blood vessels may cause the retina to detach from the back of the eye.

In some embodiments, the complement-mediated ocular disorder is diabetic macular edema (DME). DME is an eye condition which can occur in people living with both type 1 and type 2 diabetes. DME occurs when damaged blood vessels in the retina are left untreated as a result of having diabetic retinopathy. The blood vessels leak fluid into the retina, which causes swelling in the center part of the eye (macula) that provides the sharp vision needed for reading and recognizing faces. There are two types of DME, focal DME, which occurs because of abnormalities in the blood vessels in the eye, and diffuse DME, which occurs because of widening/swelling retinal capillaries (very thin blood vessels).

In some embodiments, the complement-mediated ocular disorder is neuromyelitis optica spectrum disorder (NMOSD). NMOSD is an autoimmune disorder in which white blood cells and antibodies primarily attack the optic nerves and the spinal cord, but may also attack the brain. The damage to the optic nerves produces swelling and inflammation that cause pain and loss of vision. NMOSD is characterized by inflammation/demyelination of nerve fibers as a result of a complex and poorly understood interplay of genetic and environmental factors.

The cause of NMO in the majority of cases is due to a specific attack on the aquaporin-4 (AQP4) water channel located within the optic nerves and spinal cord. Aquaporins (AQPs) are proteins that transport water across cell membranes. More than 70% of NMO and NMOSD patients test positive for an antibody biomarker in the blood called the NMO-IgG or anti-AQP4 antibody. There are two types of NMO: relapsing form, which has periodic flare-ups, with some recovery in between, where women are far more likely to have this form than men; and monophasic form, which involves a single attack that lasts a month or two, and occurs equally among men and women.

In some embodiments, the complement-mediated ocular disorder is retinal vein occlusion (RVO). Retinal vein occlusion is a blockage of the small veins that carry blood away from the retina. Retinal vein occlusion is most often caused by hardening of the arteries (atherosclerosis) and the formation of a blood clot. There are two types of RVO: branch (BRVO) where the blockage occurs in one of the smaller branch veins; and central (CRVO), wherein the blockage occurs in the main retinal vein.

In some embodiments, the complement-mediated disorder is, but is not limited to, glaucoma, diabetic retinopathy, blistering cutaneous diseases (including bullous pemphigoid, pemphigus, and epidermolysis bullosa), ocular cicatricial pemphigoid, uveitis, Behcet's uveitis, intermediate uveitis, radiation retinopathy, age-related macular degeneration, early stage age-related macular degeneration, intermediate stage age-related macular degeneration, advanced stage age-related macular degeneration, neovascular age-related macular degeneration, Wet (exudative) age-related macular degeneration, specific genotypes associated with macular degeneration, dry adult macular degeneration, retinitis pigmentosa, macular edema, diabetic macular edema, cystoid macular edema, diabetic macular edema, ocular edema, multifocal choroiditis, Vogt-Koyanagi-Harada syndrome, birdshot retinochorioditis, sympathetic ophthalmia, ocular pemphigus, nonarteritic ischemic optic neuropathy, postoperative inflammation and retinal vein occlusion, choroidal neovascularization (CNV), Type 1 choroidal neovascularization, Type 2 choroidal neovascularization, Type 3 choroidal neovascularization, idiopathic juxtafoveal telangiectasis, polypoidal choroidal vasculopathy, juxtafoveal polypoidal choroidal vasculopathy, subfovial polypoidal choroidal vasculopathy, presumed ocular histoplasmosis syndrome, ocular ischemia, subretinal neovascularization, geographic atrophy, retinal arterial occlusion, central retinal vein occlusion (CVRO), hemispheric retinal vein occlusion (HRVO), or branch retinal vein occlusion (BVRO).

In some embodiments, the ocular disorder, includes but is not limited to, glaucoma, diabetic retinopathy, blistering cutaneous diseases (including bullous pemphigoid, pemphigus, and epidermolysis bullosa), ocular cicatricial pemphigoid, uveitis, Behcet's uveitis, intermediate uveitis, radiation retinopathy, age-related macular degeneration, early stage age-related macular degeneration, intermediate stage age-related macular degeneration, advanced stage age-related macular degeneration, neovascular age-related macular degeneration, Wet (exudative) age-related macular degeneration, specific genotypes associated with macular degeneration, dry adult macular degeneration, retinitis pigmentosa, macular edema, diabetic macular edema, cystoid macular edema, ocular edema, multifocal choroiditis, Vogt-Koyanagi-Harada syndrome, birdshot retinochorioditis, sympathetic ophthalmia, ocular pemphigus, nonarteritic ischemic optic neuropathy, postoperative inflammation and retinal vein occlusion, choroidal neovascularization (CNV), Type 1 choroidal neovascularization, Type 2 choroidal neovascularization, Type 3 choroidal neovascularization, idiopathic juxtafoveal telangiectasis, polypoidal choroidal vasculopathy, juxtafoveal polypoidal choroidal vasculopathy, subfovial polypoidal choroidal vasculopathy, presumed ocular histoplasmosis syndrome, ocular ischemia, subretinal neovascularization, geographic atrophy, retinal arterial occlusion, central retinal vein occlusion (CVRO), hemispheric retinal vein occlusion (HRVO), branch retinal vein occlusion (BVRO), retinal damage in response to light exposure, retinal degeneration, retinal detachment, retinal dysfunction, retinal neovascularization (RNV), retinopathy of prematurity, pathological myopia, RPE degeneration, pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, formation of ocular drusen, chronic urticaria, Churg-Strauss syndrome, cold agglutinin disease (CAD), corticobasal degeneration (CBD), cyclitis, damage of the Bruch's membrane, Degos disease, diabetic angiopathy or focal segmental glomerulosclerosis, keratoconjunctivitis sicca, diabetic retinopathy, diabetic macula edema, Sjogren's syndrome, dry eye, scleritis, birdshot retinochoroidopathy, keratitis, sympathetic ophthalmia, Fuchs' heterochromic iridocyclitis, uveitis, episcleritis, optic neuritis, orbital pseudotumor, retinal vasculitis, ocular allergy, chronic conjunctivitis, amoebic keratitis, fungal keratitis, bacterial keratitis, viral keratitis, onchocercal keratitis, bacterial keratoconjunctivitis, viral keratoconjunctivitis, corneal dystrophic diseases, Fuchs' endothelial dystrophy, Stevens-Johnson syndrome, autoimmune dry eye diseases, environmental dry eye diseases, vasculitis, corneal neovascularization diseases, post-corneal transplant rejection prophylaxis and treatment, autoimmune uveitis, infectious uveitis, posterior uveitis (including toxoplasmosis), pan-uveitis, anterior uveitis, HLA-B27 related uveitis, herpetic keratouveitis, cytomegalovirus anterior uveitis, non-infectious uveitis, an inflammatory disease of the vitreous or retina, endophthalmitis prophylaxis and treatment, macular degeneration, proliferative and non-proliferative diabetic retinopathy, ischemic retinopathy, optic neuropathy, hypertensive retinopathy, an autoimmune disease of the retina, primary and metastatic intraocular melanoma, other intraocular metastatic tumors, open angle glaucoma, closed angle glaucoma, pigmentary glaucoma, neovascular glaucoma, astigmatism, hyperopia, presbyopia, surgery-induced edema, surgery-induced neovascularization, retinoschisis, retinal capillary occlusions, retinal angiomatous proliferation, vitreous hemorrhage, polypoidal choroidal vasculopathy, vitreomacular adhesion, retinal hypoxia, pathological myopia, dysregulated para-inflammation, chronic inflammation, chronic wound healing environment in the aging eye, carotid cavernous fistula, idiopathic occlusive arteriolitis, birdshot retinochoroidopathy, retinal vasculitis, incontinentia pigmenti, tachyphylaxis, episcleritis, idiopathic episcleritis, anterior episcleritis, or posterior episcleritis, and idiopathic Posner Schlossman syndrome.

Confirmation of Ocular Accumulation of Compounds of Formula I and Formula II

The concentration of a complement factor D-inhibitor compound as described herein can be determined using known methods. For example, plasma concentrations can be determined using standard assays, for example liquid chromatography-mass spectrometry (LC-MS/) or liquid chromatography-mass spectrometry-mass spectrometry (LC-MS/MS). Likewise, determining drug concentrations in the eye are also known.

In one embodiment, the presence and sometimes level of drug in the target ocular tissue can be assessed by its ability to treat the identified disease. When the drug is no longer present at a level to cause a therapeutic effect, it can be considered to have decreased to a sub-efficacious level.

In some embodiments, the complement factor D-inhibiting drug can be radiolabeled using standard procedures, and then assessed using known techniques. Radiolabels include but are not limited to ¹⁴C and/or, for example ²H or ³H, using detection or imaging techniques, such as positron emission tomography (PET) or single-photon emission computed tomography (SPECT).

In some embodiments, an ¹⁸F labeled compound may be particularly desirable for PET or SPECT studies. Isotopically labeled compounds of this disclosure and prodrugs thereof can generally be prepared by carrying out the procedures disclosed in the schemes or in the Examples and preparations described below by substituting a readily available isotopically labeled reagent for a non-isotopically labeled reagent. For example, in some embodiments, the concentrations of Compound 1 in the eye can be determined by using an ¹⁸F-labeled compound and the targeted imaging techniques as described by Gang Ren et al., J. Nucl. Med., 2009, 50(10), 1692-1699; LA. Alavi, J. W. Kung, H. Zhuang, Seminars in Nuclear Medicine 56-69, incorporated herein by reference. Additional techniques for using radiolabeled compounds for determination of drug concentrations using PET for imaging are also described in, for example, Sadzot et al., Synapse, 1999, 31(1), 5-12, incorporated herein by reference.

Additional method of using SPECT for imaging can be used by a skilled artisan by referring to methods disclosed by Rimpela et al., Mol Pharm., 2016, 13(9), 2977-86; Annals of Nuclear Medicine, 2019, 33(2); Bolle et al., E. Bolle et al., AX-PET: A novel PET concept with G-APD readout, Nuclear Instruments and Methods in Physics Research A 695 129-134 2012; Brooks et al., Pharm Pat Anal. 2016 January; 5(1): 17-47; Gang Ren et al., J. Nucl. Med., 2009, 50(10), 1692-1699; and Vasteenkiste et al., Murray and Nadel's Textbook of Respiratory Medicine (Sixth Edition), 2016, 1, 360-371; Rimpela et al., Advanced Drug Delivery Reviews, 2018, 126, 23-43; Rimpela et al., Molecular Pharmaceutics, 2016, 13(9); and Shin-Woo Ha et al., J Nanobiotechnol 15, 2017, 7, each of which is incorporated herein by reference.

Methods of Treatment

A method is provided for producing a depot for the delivery of an effective amount of an alternative pathway complement factor D (CFD) inhibitor of Formula I or Formula II, or a pharmaceutically acceptable salt thereof, for the treatment of a complement-mediated disorder in the human eye, without the use of an injection, implant, or medical device, wherein the CFD inhibitor is administered to the human via oral or parenteral delivery. Oral delivery is particularly preferred.

Compounds of Formula I and Formula II are capable of forming a depot within the choroid-retina pigmented epithelium (C-RPE) and the iris-ciliary body (I-CB) of the eye, which can be accumulated, and in some embodiments, through oral or parenteral administration, saturated relatively quickly, for example, within about 8 days. This depot provides a drug concentration in the C-RPE and/or I-CB that is disproportionately greater than the drug concentration found in the plasma throughout the administration period, for example greater than 2× or more, and maintains significant drug concentrations in the retina. By establishing a depot capable of delivering effective amounts of a CFD inhibitor, complement-mediated disorders affecting both the anterior and posterior of the eye can be treated without the need for depot creation through intravitreal and/or suprachoroidal injections. The depot formed provides a persistent concentration at the C-RPE and the I-CB, which, following sufficient accumulation in the depot, provides effective CFD inhibitor drug concentrations that persist between drug administrations, and provides extended delivery to the eye for at least 7 days after cessation of administration. Particular complement-mediated disorders to be treated are described throughout the specification and incorporated specifically into the methods described in this section.

As provided herein, the compounds described are administered via oral or parenteral delivery and accumulate in the choroidal-RPE tissue and iris-ciliary body (I-CB) at a disproportionate rate compared to that seen in the plasma. This allows for the ability to orally dose the compounds described herein, and, because of the accumulation, use the accumulated drug to provide continuous exposure of the drug to the eye, resulting in the extended delivery of the eye for at least 7 days after the last administered dose of the compound. In some embodiments, the extended release provides delivery from the depot for at least 7 days, 8 days, 10 days, 14 days, 21 days, 1 month, 2 months, 3 months, 4 months, 5 months, 6 months, or greater, following cessation of the last administered dose.

The compounds described herein are capable of forming a depot in the choroid-RPE and I-CB areas of the eye, which can be sufficiently accumulated over time by oral administration of the drug at less than optimal doses sufficient to initially treat a disorder if such contribution from the depot were not to occur. In some embodiments, the depot is sufficiently accumulated to provide an effective therapeutic drug concentration to the eye, in concert with the oral dose, within about 15 days of initial administration. In some embodiments, the depot is sufficiently accumulated within about 12 days, 10 days, 9 days, 8 days, 7 days, 6 days, 5 days, 4 days, or 3 days of initial administration.

In some embodiments, the compounds described herein are administered in a varying dosing schedule. For example, in some embodiments, a first dose is given over a certain time period and a second dose is given over a subsequent time period, wherein the first dose is higher and/or more frequent than the second dose. Accordingly, a first dose may be administered at a higher dosage or more frequent administration, that is, as a “ramp-up” dose wherein the dose schedule provides for the accumulation of the drug in the eye as the depot becomes near saturated. Following sufficient accumulation or near saturation, the dosing schedule can be reduced, for example, in either dosing frequency or on the amount of drug administered at each dosing. In some embodiments, the first dose may be given once a day, twice a day, or three time a day, until the depot in the eye is believed to have become sufficiently accumulated, or is some embodiments, nearly saturated. Following this period, the dose can be reduced in administration frequency, for example, the compound can be administered every other day, bi-weekly, or once a week, or, in an alternative embodiment, administered at a lower concentration. In some embodiments, the first dosing period is no greater than, 5, 6, 7, 8, 9, 10, 11, 12, 13, 14, or 15 days. In some embodiments, the second dosing period is at least 5, 10, 15, 20, or 25 days or 1, 2, 3, 4, 5, or 6 months.

In some embodiments, the compounds described herein is accumulated in the choroid-RPE and/or the iris-ciliary body at a concentration that is disproportionately greater than that seen in the plasma, allowing for the maintenance of the drug at significant levels throughout a dosing period, for example, between administrations of each dose. For example, in some embodiments, the concentration of the drug in the choroid-RPE and/or iris ciliary body is 2× greater than the concentration of the drug in the plasma between doses. In some embodiments, the concentration in the choroid-RPE and/or ciliary body is at least 2×, at least 3×, at least 4×, at least 5× times greater than the concentration in the plasma between doses. In addition, because a depot is formed, the depot provides drug to other areas of the eye, for example, the retina. In some embodiments, the retina maintains a concentration of no less than about 0.6 of the concentration observed in the plasma over the course of a dose. In some embodiments, the concentration in the retina is 0.6, 0.65, 0.7, 0.75, 0.8 or greater in the retina compared to the plasma between dosing periods.

The compounds of Formula 1 and Formula II can be administered to the subject in a dosage form that contains from about 10 mg to about 1200 mg, from about 20 mg to about 1000 mg, from about 40 mg to about 800 mg, or from about 80 mg to about 600 mg of the active compound in a unit dosage form. Examples are dosage forms with at least about 10, 15, 20, 25, 50, 75, 100, 125, 140, 150, 175, 200, 225, 250, 300, 350, 400, 450, 500, 550, 600, 650, 700, 750, 800, 900, 1000, 1100, or 1200 mg of active compound, or its salt, N-oxide, isotopic analog, or prodrug. In one embodiment, the dosage form has at least about 10 mg, 25 mg, 40 mg. 50 mg, 75 mg, 80 mg, 100 mg, 125 mg, 140 mg. 150 mg, 175 mg, 200 mg, 225 mg, 400 mg, 500 mg, 600 mg, 1000 mg, or 1200 mg of active compound, N-oxide, isotopic analog, prodrug, or its salt. The amount of active compound in the dosage form is calculated without reference to the salt. The dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein. In some embodiments, the compound is administered no more than once a day.

In some embodiments, a compound of Formula I or Formula II is administered in a regimen that provides for a first dosing schedule and a second dosing schedule, wherein the first dosing schedule comprises administering the compound in a higher amount and/or frequency than the second dosing regimen. In certain embodiments, the first dosing regimen is limited in time, for example, less than 15 days, less than 14 days, less than 13 days, less than 12 days, less than 11 days, less than 10 days, less than 9 days, less than 8 days, less than 7 days, less than 6 days, less than 5 days, less than 4 days, or less than 3 days.

In some embodiments, the administration regimen of the present disclosure comprises the oral administration of Compound 1, wherein Compound 1 is administered in a dosing regimen resulting in an AUC₍₀₋₂₄₎ of between about 2500 ng*hr/ml and 12,000 ng*hr/ml. In some embodiments, Compound 1 is administered in a dosing regimen resulting in an AUC₍₀₋₂₄₎ of between about 5,000 ng*hr/mL and 8,000 ng*hr/mL. In some embodiments, Compound 1 is administered in a dosing regimen resulting in an AUC₍₀₋₂₄₎ of between about 4,000 ng*hr/mL and 8,000 ng*hr/mL. In some embodiments, Compound 1 is administered in a dosing regimen resulting in an AUC₍₀₋₂₄₎ of between about 5,000 ng*hr/mL and 7,000 ng*hr/mL. In some embodiments, Compound 1 is administered in a dosing regimen resulting in an AUC₍₀₋₂₄₎ of less than 10,000 ng*hr/mL. In some embodiments, Compound 1 is administered in a dosing regimen resulting in an AUC₍₀₋₂₄₎ of less than 9,000 ng*hr/mL. In some embodiments, Compound 1 is administered in a dosing regimen resulting in an AUC₍₀₋₂₄₎ of less than 8,000 ng*hr/mL. In some embodiments, Compound 1 is administered in a dosing regimen resulting in an AUC₍₀₋₂₄₎ of less than 7,000 ng*hr/mL. In some embodiments, Compound 1 is administered in a dosing regimen resulting in an AUC₍₀₋₂₄₎ of less than 6,000 ng*hr/mL. In some embodiments, Compound 1 is administered in a dosing regimen resulting in an AUC₍₀₋₂₄₎ of less than 5,000 ng*hr/mL. In some embodiments, Compound 1 is administered in a dosing regimen resulting in an AUC₍₀₋₂₄₎ of less than 4,000 ng*hr/mL. In some embodiments, Compound 1 is administered in a dosing regimen resulting in an AUC₍₀₋₂₄₎ of less than 3,000 ng*hr/mL. In some embodiments, Compound 1 is administered once a day. In some embodiments, Compound 1 is administered once a day in a dose of between about 200 mg and 1000 mg. In some embodiments, Compound 1 is administered once a day in a dose of between about 200 mg and 800 mg. In some embodiments, Compound 1 is administered in a dose of less than 1000 mg. In some embodiments, Compound 1 is administered in a dose of less than 900 mg. In some embodiments, Compound 1 is administered in a dose of less than 800 mg. In some embodiments, Compound 1 is administered in a dose of less than 700 mg. In some embodiments, Compound 1 is administered in a dose of less than 600 mg. In some embodiments, Compound 1 is administered in a dose of between 500 mg and 800 mg. In some embodiments, Compound 1 is administered in a dose of less than 500 mg.

In some embodiments, the administration regimen of the present disclosure comprises the oral administration of Compound 1 at a first dosing regimen, wherein Compound 1 is administered at a daily dose of between 500 mg and 1000 mg, and a second dosing regimen, wherein Compound 1 is administered at a daily dose of less than 500 mg. In some embodiments, the first dosing regimen is administered for less than 15 days. In some embodiments, the first dosing regimen is administered for less than 8 days.

In some embodiments, the administration regimen of the present disclosure comprises the oral administration of Compound 2, wherein Compound 2 is administered in a dosing regimen resulting in an AUC₍₀₋₂₄₎ of between about 500 ng*hr/ml and 4,500 ng*hr/ml. In some embodiments, Compound 2 is administered in a dosing regimen resulting in an AUC₍₀₋₂₄₎ of between about 500 ng*hr/mL and 3,000 ng*hr/mL. In some embodiments, Compound 2 is administered in a dosing regimen resulting in an AUC₍₀₋₂₄₎ of between about 1,000 ng*hr/mL and 2,500 ng*hr/mL. In some embodiments, Compound 2 is administered in a dosing regimen resulting in an AUC₍₀₋₂₄₎ of between about 1,500 ng*hr/mL and 2,500 ng*hr/mL. In some embodiments, Compound 2 is administered in a dosing regimen resulting in an AUC₍₀₋₂₄₎ of less than 4,500 ng*hr/mL. In some embodiments, Compound 2 is administered in a dosing regimen resulting in an AUC₍₀₋₂₄₎ of less than 4,000 ng*hr/mL. In some embodiments, Compound 2 is administered in a dosing regimen resulting in an AUC₍₀₋₂₄₎ of less than 3,000 ng*hr/mL. In some embodiments, Compound 2 is administered in a dosing regimen resulting in an AUC₍₀₋₂₄₎ of less than 2,000 ng*hr/mL. In some embodiments, Compound 2 is administered in a dosing regimen resulting in an AUC₍₀₋₂₄₎ of less than 1,000 ng*hr/mL. In some embodiments, Compound 2 is administered once a day in a dose of between about 40 mg and 300 mg. In some embodiments, Compound 2 is administered once a day in a dose of between about 80 mg and 250 mg. In some embodiments, Compound 2 is administered in a dose of less than 300 mg. In some embodiments, Compound 2 is administered in a dose of less than 250 mg. In some embodiments, Compound 2 is administered in a dose of less than 200 mg. In some embodiments, Compound 2 is administered in a dose of less than 150 mg. In some embodiments, Compound 2 is administered in a dose of between 100 mg and 200 mg. In some embodiments, Compound 2 is administered in a dose of less than 100 mg.

In some embodiments, the administration regimen of the present disclosure comprises the oral administration of Compound 2 at a first dosing regimen, wherein Compound 2 is administered at a daily dose of between 150 mg and 300 mg, and a second dosing regimen, wherein Compound 2 is administered at a daily dose of less than 150 mg. In some embodiments, the first dosing regimen is administered for less than 15 days. In some embodiments, the first dosing regimen is administered for less than 8 days.

In some embodiments, the administration regimen of the present disclosure comprises the oral administration of Compound 3, wherein Compound 3 is administered in a dosing regimen resulting in an AUC₍₀₋₂₄₎ of between about 400 ng*hr/ml and 4,000 ng*hr/ml. In some embodiments, Compound 3 is administered in a dosing regimen resulting in an AUC₍₀₋₂₄₎ of between about 1,500 ng*hr/mL and 2,500 ng*hr/mL. In some embodiments, Compound 3 is administered in a dosing regimen resulting in an AUC₍₀₋₂₄₎ of between about 1,000 ng*hr/mL and 2,500 ng*hr/mL. In some embodiments, Compound 3 is administered in a dosing regimen resulting in an AUC₍₀₋₂₄₎ of between about 1,000 ng*hr/mL and 2,000 ng*hr/mL. In some embodiments, Compound 3 is administered in a dosing regimen resulting in an AUC₍₀₋₂₄₎ of less than 4,000 ng*hr/mL. In some embodiments, Compound 3 is administered in a dosing regimen resulting in an AUC₍₀₋₂₄₎ of less than 3,000 ng*hr/mL. In some embodiments, Compound 3 is administered in a dosing regimen resulting in an AUC₍₀₋₂₄₎ of less than 2,500 ng*hr/mL. In some embodiments, Compound 3 is administered in a dosing regimen resulting in an AUC₍₀₋₂₄₎ of less than 2,000 ng*hr/mL. In some embodiments, Compound 3 is administered in a dosing regimen resulting in an AUC₍₀₋₂₄₎ of less than 1,500 ng*hr/mL. In some embodiments, Compound 3 is administered in a dosing regimen resulting in an AUC₍₀₋₂₄₎ of less than 1,000 ng*hr/mL. In some embodiments, Compound 3 is administered once a day. In some embodiments, Compound 3 is administered once a day in a dose of between about 20 mg and 350 mg. In some embodiments, Compound 3 is administered once a day in a dose of between about 20 mg and 250 mg. In some embodiments, Compound 3 is administered in a dose of less than 300 mg. In some embodiments, Compound 3 is administered in a dose of less than 250 mg. In some embodiments, Compound 3 is administered in a dose of less than 200 mg. In some embodiments, Compound 3 is administered in a dose of less than 150 mg. In some embodiments, Compound 3 is administered in a dose of less than 100 mg.

In some embodiments, the administration regimen of the present disclosure comprises the oral administration of Compound 3 at a first dosing regimen, wherein Compound 3 is administered at a daily dose of between 150 mg and 300 mg, and a second dosing regimen, wherein Compound 3 is administered at a daily dose of less than 150 mg. In some embodiments, the first dosing regimen is administered for less than 15 days. In some embodiments, the first dosing regimen is administered for less than 8 days.

Pharmaceutical Preparations

A complement factor D small molecule inhibitor of Formula I or Formula II as described herein, or its salt, isotopic analog, or prodrug can be administered in an effective amount to a subject, to treat a complement-mediated ocular disorder described herein, using any suitable approach which achieves the desired therapeutic result. The amount and timing of active compound administered will, of course, be dependent on the subject being treated, the instructions of the supervising medical specialist, on the time course of the exposure, on the manner of administration, on the pharmacokinetic properties of the particular active compound, and on the judgment of the prescribing physician. Thus, because of the subject to subject variability, the dosages given below are a guideline and the physician can titrate doses of the compound to achieve the treatment that the physician considers appropriate for the subject in need. In considering the degree of treatment desired, the physician can balance a variety of factors such as age and weight of the subject, presence of preexisting disease, as well as presence of other diseases.

The complement factor D small molecule inhibitor of Formula I or Formula II, or salts thereof, as described herein, can be administered as the neat chemical, but can also be administered as a pharmaceutical composition, that includes an effective amount for a subject, typically a human, in need of such treatment of compounds as described herein. Accordingly, the disclosure provides pharmaceutical compositions comprising a dosage form in an effective amount of a compound as described herein or pharmaceutically acceptable salt thereof, together with at least one pharmaceutically acceptable carrier. The pharmaceutical composition may contain a compound or salt as the only active agent, or, in an alternative embodiment, the compound and at least one additional active agent. The pharmaceutical composition may also include a molar ratio of the active compound and an additional active agent.

The active compounds as described herein are typically administered orally in dosage unit formulations containing conventional pharmaceutically acceptable carriers. The pharmaceutical composition may be formulated as any pharmaceutically useful for oral or parenteral administration including, but not limited to, solid forms, such as pills, capsules, granules, tablets, and powders, liquid oral forms, such as solutions, syrups, elixirs, and suspension; semi-solid forms, or sublingual forms.

Some dosage forms, such as tablets and capsules, are subdivided into suitably sized unit doses containing appropriate quantities of the active components, e.g., an effective amount to achieve the desired purpose. In accordance with the presently disclosed methods, an oral administration can be in any desired form such as a solid, gel or liquid, including a solution, suspension, or emulsion.

The pharmaceutical compositions/combinations formulated for oral administration may contain any amount of a compound as described herein that achieves the desired result. For example, between 0.1 and 99 weight % (wt. %) of a compound, or at least about 5 wt. % of the compound. In certain embodiments, the composition may contain from about 25 wt. % to about 50 wt. % or from about 5 wt. % to about 75 wt. % of the compound.

The therapeutically effective dosage of any active compound described herein will be determined by the health care practitioner depending on the condition, size, and age of the subject, as well as the route of delivery.

Carriers include excipients and diluents and must be of sufficiently high purity and sufficiently low toxicity to render them suitable for administration to the subject or patient being treated. The carrier can be inert, or it can possess pharmaceutical benefits of its own. The amount of carrier employed in conjunction with a compound of Formula I or Formula II is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of a compound as described in the present disclosure.

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

The pharmaceutical composition is typically in unit dosage form suitable for single administration of a precise dosage. In certain embodiments, the unit dosage form suitable for administration of a precise dosage daily. In certain embodiments, the unit dosage form suitable for single administration of a precise dosage on alternate days. In certain embodiments, the unit dosage form suitable for administration of a precise dosage twice daily. In certain embodiments, the unit dosage form suitable for administration of a precise dosage on alternate days. In certain embodiments, the unit dosage form suitable for single administration of a precise dosage on alternate days. In certain embodiments, the unit dosage form suitable for oral administration of a precise dosage biweekly. In certain embodiments, the unit dosage form suitable for single administration of a precise dosage biweekly.

The compositions will include an effective amount of the selected drug in combination with a pharmaceutically acceptable carrier and, in addition, can include other pharmaceutical agents, adjuvants, diluents, buffers, and the like.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced herein.

Solid compositions of a similar type may be employed as fillers in soft and hard-filled gelatin capsules. Materials in this connection also include lactose or milk sugar as well as high molecular weight polyethylene glycols.

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

For oral administration, a pharmaceutical composition can take the form of a solution, liquid, emulsion, suspension, tablet, pill, capsule, powder, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc are often very useful for tableting purposes.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

When aqueous suspensions and/or elixirs are desired for oral administration, the compounds of the present disclosure can be combined with various sweetening agents, flavoring agents, coloring agents, emulsifying agents and/or suspending agents, as well as such diluents as water, ethanol, propylene glycol, glycerin and various like combinations thereof.

In addition to the active compounds or their salts described herein, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

Combination Therapies

In one embodiment, an effective amount of an active compound of Formula I or Formula II or its pharmaceutically acceptable salt or composition as described herein may be provided in combination or alternation with an effective amount of at least one additional inhibitor of the complement system or a second active compound with a different biological mechanism of action. In the description below and herein generally, whenever any of the terms referring to an active compound or its salt or composition as described herein are used, it should be understood that pharmaceutically acceptable salts, prodrugs or compositions are considered included, unless otherwise stated or inconsistent with the text.

In certain embodiments, a complement factor D small molecule inhibitor of Formula I or Formula II, as described herein, may be provided in combination or alternation with at least one additional therapeutic agent, for the treatment of a disorder listed herein.

In certain embodiments, a complement factor D small molecule inhibitor of Formula I or Formula II, as described herein, may be orally administered in combination with a complement 5 (C5) inhibitor. C5 inhibitors are known in the art. In some embodiments, the C5 inhibitor is a monoclonal antibody targeting C5. In some embodiments, the C5 inhibitor is eculizumab (SOLIRIS®, Alexion Pharmaceuticals, Inc., Boston, Mass., see, e.g., U.S. Pat. No. 9,352,035), or a biosimilar molecule thereof. In some embodiments, the C5 inhibitor is ravulizumab (ULTOMIRIS®, Alexion Pharmaceuticals, Inc. Boston, Mass., see, e.g., U.S. Pat. Nos. 9,371,377; 9,079,949 and 9,663,574), or a biosimilar thereof.

In some embodiments, the C5 inhibitor may be, but is not limited to: a recombinant human minibody, for example Mubodina® (monoclonal antibody, Adienne Pharma and Biotech, Bergamo, Italy; see U.S. Pat. No. 7,999,081); coversin (nomacopan, Akari Therapeutics; see e.g. Penabad et al. Lupus, 2012, 23(12):1324-6); LFG316 (monoclonal antibody, Novartis, Basel, Switzerland, and Morphosys, Planegg, Germany; see U.S. Pat. Nos. 8,241,628 and 8,883,158); ARC-1905 (pegylated RNA aptamer, Ophthotech, Princeton, N.J. and New York, N.Y.; see Keefe et al., Nature Reviews Drug Discovery, 9, 537-550); RA101348 and zilucoplan (macrocyclic peptides, Ra Pharmaceuticals, Cambridge, Mass.); SOBI002 (affibody, Swedish Orphan Biovitrum, Stockholm, Sweden); cemdisiran (Alnylam Pharmaceuticals, Cambridge, Mass.); ARC1005 (aptamers, Novo Nordisk, Bagsvaerd, Denmark); SOMAmers (aptamers, SomaLogic, Boulder, Colo.); SSL7 (bacterial protein toxin, see, e.g. Laursen et al. Proc. Natl. Acad. Sci. U.S.A., 107(8):3681-6); MED17814 (monoclonal antibody, MedImmune, Gaithersburg, Md.); aurin tricarboxylic acid; aurin tricarboxylic acid derivatives (Aurin Biotech, Vancouver, BC, see U.S. Patent Appl. Pub. 2013/003592); crovalimab (RG6107/SKY59; anti-C5 recycling antibody, Roche Pharmaceuticals, Basel, Switzerland); ALXN5500 (monoclonal antibodies, Alexion Pharmaceuticals, New Haven, Conn.); TT30 (fusion protein, Alexion Pharmaceuticals, Inc., Boston, Mass.); prozelimab (REGN3918; monoclonal antibody, Regeneron, Tarrytown, N.Y.); ABP959 (eculizumab biosimilar, Amgen, Thousand Oaks, Calif.); BCD-148 (Biocad); and SB-12 (Samsung Bioepis Co., Ltd.); and combinations thereof.

In certain embodiments, a complement factor D small molecule inhibitor of Formula I or Formula II, as described herein, may be orally administered in combination with C3 inhibitors that include compstatin and/or a compstatin analog. Compstatin and compstatin analogs are known and are found to be useful inhibitors of C3, see U.S. Pat. Nos. 9,056,076; 8,168,584; 9,421,240; 9,291,622; 8,580,735; 9,371,365; 9,169,307; 8,946,145; 7,989,589; 7,888,323; 6,319,897; and US Patent Appl. Pub. Nos. 2016/0060297; 2016/0015810; 2016/0215022; 2016/0215020; 2016/0194359; 2014/0371133; 2014/0323407; 2014/0050739; 2013/0324482; and 2015/0158915. In some embodiments, the compstatin analog having the amino acid sequence ICVVQDWGHHCRT (SEQ. ID. NO. 1). In another embodiment, the C3 inhibitor is a compstatin analog. In some embodiments, the compstatin analog is 4(1MeW)/APL-1 of the sequence Ac-ICV(1-mW)QDWGAHRCT (SEQ. ID. NO. 2), wherein Ac is acetyl and 1-mW is 1-methyltryptophan. In another embodiment, the compstatin analog is Cp40/AMY-101, which has an amino acid sequence yICV(1 mW)QDW-Sar-AHRC-mI (SEQ. ID. NO. 3), wherein y is D-tyrosine, 1 mW is 1-methyltryptophan, Sar is sarcosine, and mI is N-methylisoleucine. In yet another embodiment, the compstatin analog is PEG-Cp40, having the amino acid sequence PEG-yICV(1 mW)QDW-Sar-AHRC-mI (SEQ. ID. NO. 4), wherein PEG is polyethyleneglycol (40 kDa), y is D-tyrosine, 1 mW is 1-methyltryptophan, Sar is sarcosine, and mI is N-methylisoleucine. In another embodiment, the compstatin analog is 4(1MeW)POT-4. 4(1MeW)POT-4 was developed by Potentia. In another embodiment, the compstatin analog is AMY-201. AMY-201 was developed by Amyndas Pharmaceuticals.

In certain embodiments, a complement factor D small molecule inhibitor of Formula I or Formula II, as described herein, may be orally administered in combination with C3 inhibitors that include, but are not limited to: H17 (monoclonal antibody, EluSys Therapeutics, Pine Brook, N.J.); mirococept (CR1-based protein); sCR1 (CR1-based protein, Celldex, Hampton, N.J.); TT32 (CR-1 based protein, Alexion Pharmaceuticals, Inc., Boston, Mass.); HC-1496 (recombinant peptide); CB 2782 (enzyme, Catalyst Biosciences, South San Francisco, Calif.); APL-2 (pegylated synthetic cyclic peptide, Apellis Pharmaceuticals, Crestwood, Ky.); or combinations thereof.

In some embodiments, a compound of the present disclosure can be combined with CFB inhibitors that include, but are not limited to: anti-FB SiRNA (Alnylam Pharmaceuticals, Cambridge, Mass.); TA106 (monoclonal antibody, Alexion Pharmaceuticals, Inc., Boston, Mass.); LNP023 (small molecule, Novartis, Basel, Switzerland); SOMAmers (aptamers, SomaLogic, Boulder, Colo.); bikaciomab (Novelmed Therapeutics, Cleveland, Ohio); complin (see, Kadam et al., J. Immunol. 2010, DOI:10.409/jimmunol.10000200); Ionis-FB-L_{Rx}, (ligand conjugated antisense drug, Ionis Pharmaceuticals, Carlsbad, Calif.); or a combination thereof. In another embodiment, CFB inhibitors that can be combined with a compound of the present disclosure include those disclosed in PCT/US17/39587. In another embodiment, CFB inhibitors that can be combined with a compound of the present disclosure as described herein include those disclosed in PCT/US17/014458. In another embodiment, CFB inhibitors that can be combined with a compound of the present disclosure as described herein include those disclosed in U.S. Patent Appl. Pub. No. 2016/0024079; PCT Int. Appl. WO 2013/192345; PCT Int. Appl. WO 2013/164802; PCT Int. Appl. WO 2015/066241; and PCT Int. Appl. WO 2015/009616 (assigned to Novartis AG).

In certain embodiments, a complement factor D small molecule inhibitor of Formula I or Formula II, as described herein, may be orally administered in combination with a complement factor H inhibitor.

In certain embodiments, a complement factor D small molecule inhibitor of Formula I or Formula II, as described herein may be administered in combination with another complement Factor D inhibitor.

In certain embodiments, a complement factor D small molecule inhibitor of Formula I or Formula II, as described herein may be administered in combination with a vascular endothelial growth factor (VEGF) inhibitor/antagonist, VEGF antibodies, VEGF antibody fragments. Non-limiting examples of anti-VEGF agents include, but are not limited to, aflibercept (Eylea*; Regeneron Pharmaceuticals); ranibizumab (Lucentis®: Genentech and Novartis); pegaptanib (Macugen®; OSI Pharmaceuticals and Pfizer); and Bevacizumab (Avastin; Genentech/Roche). Other non-limiting examples of anti-VEGF agents include, but are not limited to, lapatinib (Tykerb); sunitinib (Sutent); axitinib (Inlyta); pazopanib; sorafenib (Nexavar); ponatinib (Inclusig); regorafenib (Stivarga); Cabozantinib (Abometyx; Cometriq); vendetanib (Caprelsa); ramucirumab (Cyramza); lenvatinib (Lenvima); ziv-aflibercept (Zaltrap); cediranib (Recentin); anecortane acetate, squalamine lactate, and corticosteroids, including, but not limited to, triamcinolone acetonide.

In certain embodiments, a complement factor D small molecule inhibitor of Formula I or Formula II, as described herein may be administered in combination with ranibizumab.

In certain embodiments, a complement factor D small molecule inhibitor of Formula I or Formula II, as described herein may be administered in combination with pegaptanib.

In certain embodiments, an active compound or its salt or composition as described herein as described herein can be used in combination or alternation with a factor D inhibitor compound described in Biocryst Pharmaceuticals U.S. Pat. No. 6,653,340; Novartis PCT patent publication WO2012/09310, WO2014/002051, WO2014/002052, WO2014/002053, WO2014/002054, WO2014/002057, WO2014/002058, WO2014/002059, WO2014/005150, WO2014/009833, WO 2013/164802, WO 2015/009616, WO 2015/066241; Lifesci Pharmaceuticals PCT patent publication WO2017/098328 and WO2018/015818; Bristol-Myers Squibb PCT patent publication WO2004/045518; Japan Tobacco Inc. PCT patent publication WO1999/048492; Ferring B. V. and Yamanouchi Pharmaceutical Co. LTD. PCT patent publication WO1993/020099; Alexion Pharmaceuticals PCT patent publication WO1995/029697; or Patent filings that disclose complement factor D inhibitors are described in U.S. Pat. Nos. 9,598,446; 9,643,986; 9,663,543; 9,695,205; 9,732,103; 9,732,104; 9,758,537; 9,796,741; 9,828,396; 10,000,516; 10,005,802; 10,011,612; 10,081,645; 10,087,203; 10,092,584; 10,100,072; 10,138,225; 10,189,869; 10,106,563; 10,301,336; and 10,287,301; International Publication Nos. WO2019/028284; WO2018/160889; WO2018/160891; WO2018/160892; WO2017/035348; WO2017/035349; WO 2017/035351; WO 2017/035352; WO 2017/035353; WO 2017/035355; WO2017/035357; WO2017/035360; WO2017/035361; WO2017/035362; WO2017/035415; WO2017/035401; WO2017/035405; WO2017/035413; WO2017/035409; WO2017/035411; WO2017/035417; WO2017/035408 WO2015/130784; WO2015/130795; WO2015/130806; WO2015/130830; WO2015/130838; WO2015/130842; WO2015/130845; and WO2015/130854; and U.S. Patent Publication Nos. US 2016/0361329; US 2016/0362432; US 2016/0362433; US 2016/0362399; US 2017/0056428; US 2017/0057950; US 2017/0057993; US 2017/0189410; US 2017/0226142; US 2017/0260219; US 2017/0298084; US 2017/0298085; US/2018-0022766; US 2018/0022767; US 2018/0072762; US 2018/0030075; US 2018/0169109; US 2018/0177761; US 2018/0179185; US 2018/0179186; US 2018/0179236; US 2018/0186782; US 2018/0201580; US 2019/0031692; US 2019/0048033; US 2019/0144473; and US 2019/0211033 all owned by Achillion Pharmaceuticals, Inc, now Alexion Pharmaceuticals, Inc. Each of the above references are incorporated herein by reference.

Certain embodiments provide a method of treating or preventing age-related macular degeneration (AMD) by administering to a subject in need thereof an effective amount of an active compound of Formula I or Formula II, or its salt or composition as described herein in combination with an anti-VEGF agent. Non-limiting examples of anti-VEGF agents include, but are not limited to, aflibercept (Eylea*; Regeneron Pharmaceuticals); ranibizumab (Lucentis®: Genentech and Novartis); and pegaptanib (Macugen®; OSI Pharmaceuticals and Pfizer); Bevacizumab (Avastin; Genentech/Roche); anecortane acetate, squalamine lactate, and corticosteroids, including, but not limited to, triamcinolone acetonide.

### EXAMPLES

### Example 1. Tissue Distribution Via Quantitative Whole-Body Autoradiography in Male Long-Evans and Wistar Han Rats Following a Single Administration of [¹⁴C] Compound 1

This study was conducted to determine the tissue distribution of drug-related radioactivity following a single oral (PO) (20 mg/kg) or intravenous (IV) (5 mg/kg) administration of [¹⁴C] Compound 1 to male pigmented and male albino rats using quantitative whole-body autoradiography (QWBA).

Ten adult male pigmented Long-Evans (LE) rats (Group 1), 1 adult male LE rat (Group 2), and 3 albino Wistar Han (WH) rats (Group 3) received a single oral 20 mg/kg (Groups 1 and 3) or IV 5 mg/kg (Group 2) dose of [¹⁴C] Compound 1 in polyethylene glycol 400 in 0.9% saline (50:50, w/w). The formulation was prepared at a target dose of 5 mg (IV) or 20 mg (PO) [¹C] Compound 1/kg body weight and approximately 25 (IV) and 100 (PO) μCi/kg (at a concentration of 5 mg/mL and dosing volumes of 1 and 4 mL/kg for IV and PO, respectively) (See Table 1).

TABLE 1
Group Designations and Dose Information

| | | | | Target | Target | Target | Target |
|---|---|---|---|---|---|---|---|
| | | | No. | Dose | Dose | Dose | Radio- |
| | | | of | Level | Volume | Conc. | activity |
| Group | | Dose | Animals, | (mg/ | (mL/ | (mg/ | Level |
| Number | Strain | Route | Sex | kg) | kg) | mL) | (μCi/kg) |
| 1 | LE | PO | 10 M | 20 | 4 | 5 | 100 |
| 2 | LE | IV | 1 M | 5 | 1 | 5 | 25 |
| 3 | WH | PO | 3 M | 20 | 4 | 5 | 100 |

QWBA = quantitative whole-body autoradiography;
LE = Long-Evans rats;
WH = Wistar Han rats;
M = Male

Group 1 rats were euthanized at (n=1/time point) 1 hour (h), 2 h, 4 h, 8 h, 24 h, 72 h, 168 h, 336 h, 504 h, and 672 h post-dose. The Group 2 rat was euthanized at 5 min post-dose and Group 3 rats were euthanized (n=1/time point) at 1 h, 24 h, and 168 h post-dose (See Table 2). The carcasses were frozen in hexane dry-ice and stored at or below −20° C. prior to processing for QWBA. Each carcass was embedded, cut into sagittal sections, and mounted for QWBA. Concentrations of radioactivity were expressed as μCi/g and converted to μg equivalents of Compound 1 per gram of matrix (μg equiv/g) using the specific activity of 4.7616 μCi/mg of the administered formulated [¹⁴C] Compound 1. The lower limit of quantitation was 0.105 μg equiv/g.

TABLE 2
Sample Collection Summary

| Group Number | Plasma and Frozen Carcass for QWBA |
|---|---|
| 1 | n = 1/time point 1, 2, 4, 8, 24, 72, |
| | 168, 336, 504, and 672 h |
| 2 | n = 1/time point at 5 min |
| 3 | n = 1/time point at 1, 24, and 168 h |

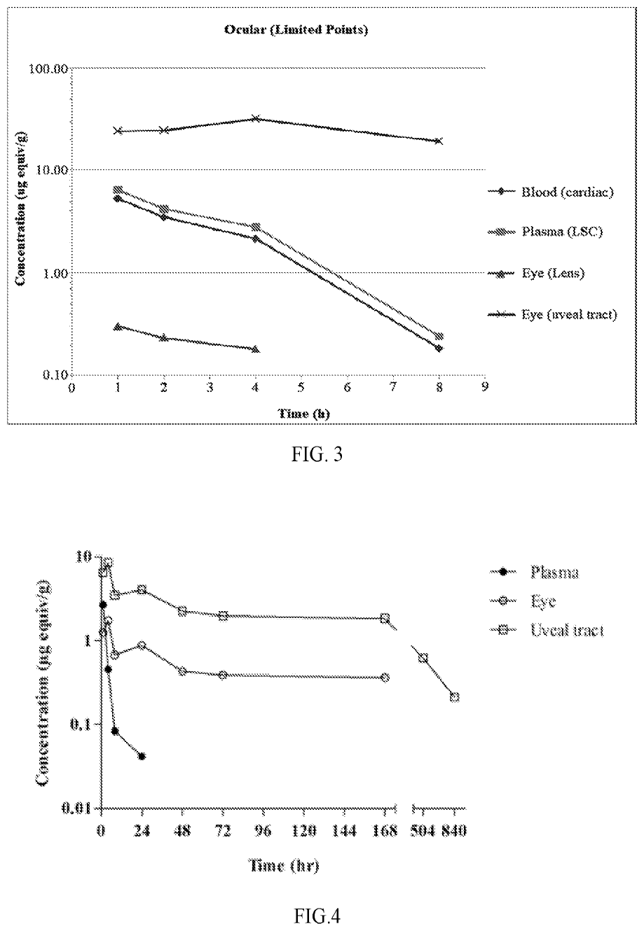

Male LE Rats after Oral Dosing: Drug-derived radioactivity was rapidly absorbed and widely distributed to tissues of male LE rats, with concentrations present in most tissues at 1 h through 8 h post-dose. Most tissue concentrations were BQL at 24 h post-dose, except for liver, pigmented skin, intestinal contents, and eye (uvea). Only eye (uvea) and pigmented skin had quantifiable concentrations at 672 h (FIGS. 2 and 3).

Most tissues of pigmented rats had concentrations that were similar to or higher than blood concentrations. The highest concentration in blood was observed at 1 h post-dose (5.28 μg equiv/g), and concentrations were detectable through 8 h post-dose (0.184 μg equiv/g). Maximum concentrations (Cₘₐₓ) in most tissues of male LE rats were observed at 1 h (37 of 40 tissues) post-dose. The highest concentrations of radioactivity in tissues of male LE rats (>20.0 μg equiv/g) at Tₘₐₓ were found in stomach (374 μg equiv/g), small intestine (77.9 μg equiv/g), urinary bladder (54.2 μg equiv/g), liver (52.0 μg equiv/g) adrenal gland cortex (33.5 μg equiv/g), and white adipose (21.1 μg equiv/g). Most tissues had concentrations that ranged between 4.00 and 12.0 μg equiv/g at Tax. The tissues with the lowest concentrations (<1.00 μg equiv/g) observed at Tₘₐₓ were brain, spinal cord, bone, testis, and the lens of the eye. The highest overall concentrations of radioactivity at Tₘₐₓ in male pigmented rats were found in the alimentary canal contents (342 to 834 μg equiv/g), bile (232 μg equiv/g at 1 h) and urine (66.2 μg equiv/g at 4 h), which suggested that renal and biliary excretion, and passage through the alimentary canal were the routes of eliminations of [¹⁴C] Compound 1-derived radioactivity after an oral dose.

Tissue concentrations versus time profiles of male LE rats showed that most tissues had a rapid distribution phase over the first 1 h followed by a 4-24 h elimination phase, and most tissues were BQL at 24 h (37 of 40). Detectable concentrations were found in pigmented skin and eye uvea at 672 h post-dose, which had t_{1/2} values of 472 h and 576 h, respectively. The liver also had a relatively long t_{1/2} of 48.5 h. The t_{1/2} was calculated for 29 of 40 other tissues and ranged between 1.1 h (white adipose, kidney medulla, and mammary gland region) and 3.0 h (seminal vesicle). The t_{1/2} of remaining tissues could not be determined due to insufficient time point data and/or resulting r² values for the concentration-time curves, which were outside the ≤0.85 criteria for determination of reliable t_{1/2} values.

The male LE rat tissues with the highest tissue to blood AUCₐₗₗ ratios were: eye uvea (101.5), stomach (39.8), pigmented skin (34.1), liver (13.8), intestinal tissues (5.2 to 7.4), adrenal gland (3.7 to 5.2), urinary bladder (4.9), kidney cortex and medulla (4.8 and 3.4, respectively), white adipose (3.5), salivary gland (3.2), brown adipose (2.9), mammary gland region (2.4), and thyroid (2.0).

Male LE Rats after IV Dosing: Drug-derived radioactivity was widely distributed to tissues of male LE rats at 5 min after an IV dose (at 5 mg/kg), with concentrations present in all tissues at 5 min post-dose. Tissue concentrations observed in this rat were similar to Group 1 rats given a single oral dose of 20 mg/kg and analyzed at 1 h post-dose.

Male WH Rats after Oral Dosing: Patterns of distribution in albino male WH rats were similar to that observed in pigmented male rats, but tissue concentrations observed in the albino rats were generally lower than those for pigmented LE rats at the same time point (i.e., 1 h). The highest concentration in blood of albino rats was 2.77 μg equiv/g at 1 h post-dose. The highest tissue concentrations observed in albino rats were observed at 1 h post-dose for all tissues; however only 1 early time point was observed so the actual Tₘₐₓ/Cₘₐₓ may not have been examined. The concentration of radioactivity in the uvea of the non-pigmented eye and non-pigmented skin at Tₘₐₓ were 1.68 and 2.98 μg equiv/g, respectively, and both decreased to BQL at 24 h and 168 h respectively. The observed differences in concentration between Group 1 pigmented and Group 3 non-pigmented tissues indicate that there was a specific association of [¹⁴C] Compound 1-derived radioactivity with melanin, but that it was reversible.

### Example 2. Tissue Distribution Via Quantitative Whole-Body Autoradiography in Male Long-Evans and Wistar Han Rats Following a Single Administration of [¹⁴C] Compound 2

The study was conducted to determine the tissue distribution of [¹⁴C] Compound 2-derived radioactivity following a single oral gavage (PO) administration of 20 mg/kg (100 μCi/kg) of [¹⁴C] Compound 2 to male Long-Evans and male Wistar Hannover rats or following a single intravenous (IV) administration of 5 mg/kg (100 μCi/kg) of [¹⁴C] Compound 2 to a male Long-Evans rat using quantitative whole body autoradiography (QWBA).

A total of ten male Long-Evans and three male Wistar Hannover rats were administered a single PO dose of [¹⁴C] Compound 2 at 20 mg/kg (100 μCi/kg) formulated in 0.5% hyrdroxypropyl methylcellulose (HPMC) (w/v) with 0.1% Tween 80 (v/v) in water. One male Long-Evans rat was administered a single IV dose of [¹⁴C] Compound 2 at 5 mg/kg (100 μCi/kg) formulated in polyethylene glycol (PEG) 400 in saline (70/30 w/w). (See Table 3). One animal per group per strain was anesthetized by inhalation of isoflurane gas at designated time points post-dose, followed by blood collection via cardiac puncture and euthanasia by isoflurane overdose. Each carcass was then frozen in a dry ice/hexane bath. Blood and plasma samples were assayed for total radioactivity (TRA) concentrations by liquid scintillation counting (LSC). The tissue concentrations of TRA were determined using validated QWBA techniques. Pharmacokinetic parameters of derived radioactivity in blood, plasma, and tissues were calculated, to the extent possible, using Phoenix© WinNonlin® (Version 6.3, Pharsight Corporation; Mountain View, Calif.).

TABLE 3
Study Design

| | | | # | | | | Collection Matrices |
|---|---|---|---|---|---|---|---|
| | Route | | Animals/ | | | | and Time |
| | of | Study | Gender - | Dose Level | | | Points/Intervals |
| Group | Administration | Design | Type | mg/kg | μCi/kg | mL/kg | Post-Dose |
| 1 | Oral (gavage) | Tissue | 7 + 3 M | 20 | 100 | 5 | Blood/Plasma and |
| | | Distribution^{a} | LE | | | | Carcass for QWBA: |
| | | | Intact | | | | 1, 4, 8, 24, 48, 72 |
| | | | | | | | and 168 h post-dose |
| | | | | | | | 504, 840, and 1008 h |
| | | | | | | | post-dose (additional |
| | | | | | | | time points)^{b} |
| 2 | Oral (gavage) | Tissue | 3 M | 20 | 100 | 5 | Blood/Plasma and |
| | | Distribution^{a} | WH | | | | Carcass for |
| | | | Intact | | | | QWBA: 1, 24, and |
| | | | | | | | 168 h post-dose |
| 3 | Intravenous | Tissue | 1 M | 5 | 100 | 2 | Blood/Plasma and |
| | (bolus) | Distribution^{a} | LE | | | | Carcass for QWBA: |
| | | | Intact | | | | 0.083 h post-dose |

LE: Long Evans,
WH: Wistar Hannover
^{a}1 animal per time point, terminal bleeds by cardiac puncture under CO₂-induced anesthesia and plasma separated by chilled centrifugation
^{b}The 504 and 840 h animals were subject to QWBA procedures since it was determined that a significant amount of radioactivity remained at 168 h post-dose. Sponsor approval was obtained to use the additional carcasses for QWBA. The 1008 h animal was not subject to QWBA procedures since it was determined that there was not a significant amount of radioactivity remaining at 840 h post-dose. Sponsor approval was obtained to not use the additional carcass for QWBA.

Maximum concentration (Cₘₐₓ) values of 2.67 μg equiv/g and 1.66 μg equiv/g were reached in plasma and blood, respectively, at 1 hour (h) post-dose, following a single PO dose of 20 mg/kg (100 μCi/kg) [¹⁴C] Compound 2 to male Long-Evans rats. The total plasma radioactivity decreased steadily after Tₘₐₓ, and was below the quantifiable limit (BLQ) at 48 h post-dose. Blood concentrations were slightly lower than plasma concentrations at the earliest sampled time points, higher at the later sampled time points, and reached BLQ levels at 72 h post-dose. The majority of tissues had Tₘₐₓ values at 1 h post-dose and were BLQ by 24 h post-dose, and all tissues were BLQ at 168 h post-dose, except the eye and uveal tract, which had concentrations present much higher than the lower limit of quantification (0.0101 μg equiv/g tissue) through 840 h post-dose (0.212 μg equiv/g tissue). The majority of tissues had tissue:plasma AUC₀₋ₜ ratio greater than 1.00.

Cₘₐₓ values of 4.91 μg equiv/g and 3.02 μg equiv/g were reached in plasma and blood, respectively, at 1 h post-dose, following a single PO dose of 20 mg/kg (100 μCi/kg) [¹⁴C] Compound 2 to male Wistar Hannover rats. The total plasma radioactivity was BLQ at 168 h post-dose. All tissues had Tₘₐₓ values at 1 h post-dose. The majority of tissues were BLQ by 24 h post-dose, and all tissues were BLQ at 168 h, except the liver. There are too few time points to accurately determine AUC for the WH rat tissues, however, the tissue to plasma concentration ratios were split at the 1 h post-dose time point with 26 tissue with a ratio <1.0 (ranging from 0.04 in brain to 0.96 in cecum mucosa) and 21 tissue with a tissue with a ratio ≥1.0 (ranging from 1.00 in stomach wall non-glandular to 12.6 kidney medulla), which may indicate the radioactivity is still distributing at 1 h post-dose. At 24 h post-dose, the tissue to plasma ratios for tissues above the LLOQ were generally all >1.0 (ranging from 2.90 in bone marrow to 100 stomach wall non-glandular), indicating that the radioactivity in tissue is likely not associated with the plasma and is out of the distribution phase.

Concentration values of 3.91 μg equiv/g and 2.43 μg equiv/g were reached in plasma and blood, respectively, at 0.083 h post-dose, following a single IV dose of 5 mg/kg (100 μCi/kg) [¹⁴C] Compound 2 to one male Long-Evans rat. The distribution following a single IV dose of 5 mg/kg (100 μCi/kg) [¹⁴C] Compound 2 was widespread, and concentrations were found in all sampled tissues. The lowest tissue concentrations were in brain (0.201 μg equiv/g tissue) and lens (0.0486 μg equiv/g tissue). The highest tissue concentrations were in the adrenal gland (10.8-19.2 μg equiv/g tissue) and small intestine wall (8.18 μg equiv/g tissue). Concentrations in the contents of the gastrointestinal tract were also high (0.292-21.1 μg equiv/g tissue) at 0.083 h following IV administration, indicating that biliary excretion was a likely component of [¹⁴C] Compound 2-derived radioactivity elimination.

In summary, [¹⁴C] Compound 2-derived radioactivity was quickly absorbed and distributed to tissues and the elimination from the body was fast following a single PO dose of 20 mg/kg (100 μCi/kg) of [¹⁴C] Compound 2 to male Long-Evans and Wistar Hannover rats. Regardless of strain, most tissues were BLQ at 24 h post-dose, and by 168 h, all tissues were BLQ except the eye and uveal tract in Long-Evans rats. The endocrine and metabolic/excretory tissues, as well as the tissues of the gastrointestinal tract contained the highest distribution of [¹⁴C] Compound 2-derived radioactivity. No measurable concentrations of radioactivity were found in the central nervous system of Long-Evans rats following a PO dose, and a low level of radioactivity was found in the central nervous system of the Wistar Hannover rat sacrificed at 1 h pose-dose (0.179 μg equiv/g tissue) which is approximately 18 times the LLOQ. In addition, there was significant distribution and prolonged retention (t1/2>215 h) of radioactivity to the melanin-containing tissues, such as the uveal tract, in the Long-Evans rats following a single PO dose of 20 mg/kg (100 μCi/kg) of [¹⁴C] Compound 2, indicating that [¹⁴C] Compound 2-derived radioactivity could be potentially binding to melanin (See FIG. 4). However, the exposure to [¹⁴C] Compound 2-derived radioactivity in pigmented skin was comparable to the non-pigmented skin. The concentrations of radioactivity in uveal tract continued to reduce over time, and that trend is expected to continue following the last sampled time point (840 h post-dose).

The distribution at 0.083 h following a single IV dose of 5 mg/kg (100 μCi/kg) of [¹⁴C] Compound 2 to a male long Evans rat was widespread, and concentrations were found in all sampled tissues. Concentrations in the contents of the gastrointestinal tract were high (0.292-21.1 μg equiv/g tissue), indicating that biliary excretion was a likely component of [¹⁴C] Compound 2-derived radioactivity elimination. The low level of radioactivity found in the central nervous system (0.201 μg equiv/g tissue) is approximately 8 times the LLOQ.

In summary, [¹⁴C] Compound 2-derived radioactivity was quickly absorbed and distributed to tissues and the elimination from the body was fast following a single PO dose of 20 mg/kg (100 μCi/kg) of [¹⁴C] Compound 2 to male Long-Evans and Wistar Hannover rats. Regardless of strain, most tissues were BLQ at 24 h post-dose, and by 168 h, all tissues were BLQ except the eye and uveal tract in Long-Evans rats. The endocrine and metabolic/excretory tissues, as well as the tissues of the gastrointestinal tract contained the highest distribution of [¹⁴C] Compound 2-derived radioactivity, regardless of strain or dose route. No measurable concentrations of radioactivity were found in the central nervous system of Long-Evans rats following a PO dose, and a low level of radioactivity was found in the central nervous system of a Wistar Hannover rat at 1 h after the PO dose. In addition, there was significant distribution and prolonged retention (t1/2>215 h) of radioactivity to the melanin-containing tissues, such as the uveal tract, in the Long-Evans rats following a single PO dose of 20 mg/kg (100 μCi/kg) of [¹⁴C] Compound 2, indicating that [¹⁴C] Compound 2-derived radioactivity could be potentially binding to melanin. However, the exposure to [¹⁴C] Compound 2-derived radioactivity in pigmented skin was comparable to the non-pigmented skin. The concentrations of radioactivity in uveal tract continued to reduce over time and that trend is expected to continue and complete clearance of radioactivity should be expected past the 840 h time point.

The distribution at 0.083 h following a single IV dose of 5 mg/kg (100 μCi/kg) [¹⁴C] Compound 2 to a Long Evans rat was widespread and concentrations were found in all sampled tissues. Concentrations following a single IV dose of 5 mg/kg (100 μCi/kg) [¹⁴C] Compound 2 in the contents of the gastrointestinal tract were also high (0.292-21.1 μg equiv/g tissue), indicating that biliary excretion was a likely component of [¹⁴C] Compound 2-derived radioactivity elimination. A low level of radioactivity was found in the central nervous system.

### Example 3. Ocular Tissue Distribution of Compound 1 Following 15 mg/kg Single Dose and BID Oral Administration in Rabbits

This study was conducted to evaluate the ocular tissue and plasma pharmacokinetics, ocular tissue distribution, and tolerability of Compound 1 following a single dose 15 mg/kg oral gavage in Dutch Belted and New Zealand White rabbits and twice daily (BID) 15 mg/kg oral gavage in Dutch Belted rabbits. Study design including group designation and dose levels are outlined in Table 4. Fourteen New Zealand White and fourteen Dutch Belted rabbits received a single 15 mg/kg dose of Compound 1. Fourteen Dutch Belted rabbits were dosed 15 mg/kg for 14 consecutive days twice a day (BID) with a single morning dose of Compound 1 on Day 15. Fifteen Dutch Belted and 5 New Zealand White rabbits were used as un-dosed controls. Compound 1 was formulated as a suspension in 0.5% hydroxypropyl methylcellulose (HPMC) with 0.1% Tween 80. The dose volume was 5 mL/kg/dose for all groups. Safety was assessed with toxicological and ophthalmic examinations including slit lamp biomicroscopy, indirect ophthalmoscopy, rebound tonometry, and ultrasonic corneal pachymetry.

The concentrations of Compound 1 in plasma and tissue homogenate samples were analyzed by a LC-MS/MS method on a Sciex API 4000 Q-Trap mass spectrometer using turbo ion spray with MRM monitoring in the positive mode. Analyst® software (Version 1.6.2) was used to capture the LC-MS/MS data and integrate the peak areas. Calibration standards and QCs were prepared in a blank solution of 75%:25% acetonitrile:water (v:v), ranging from 10 ng/mL to 10,000 ng/mL. Matrix matched standard and QC samples were prepared by mixing 2 μL of standard or QC sample above with 18 μL of appropriate ocular tissue or plasma. The final calibration standards ranged from 1 ng/mL to 1000 ng/mL. All plasma, retina, choroid-RPE, optic nerve, and vitreous humor samples were analyzed in a single run. Iris-ciliary body samples were analyzed in two runs due to a need for sample dilution. QCs for Compound 1 were prepared at 3, 30, 500, and 800 ng/mL. Dilution QCs were prepared at 5000 ng/mL where appropriate. Two sets of standards were used to bracket the analytical run at the beginning and end of the run. Three sets of QCs were included the analytical runs. All concentrations in ocular matrices are reported as ng/g based on the assumed density of 1 g/mL.

TABLE 4
Group Designations and Dose Levels
EXPERIMENTAL DESIGN
Study Design
Group Designations and Dose Levels

| | | | | Target | Target | |
|---|---|---|---|---|---|---|
| | Number | | Dose | Dose | Dose | |
| | of Male | Dose | Concentration | Level | Volume | Sample |
| Group | Animals | Route | (mg/mL) | (mg/kg) | (mL/kg/dose) | Collection |
| 1 | 14 NZW | Oral^{a} | 3 | 15 | 5 | Blood and |
| | | | | | | Ocular tissues |
| 2 | 14 DB | Oral^{a} | 3 | 15 | 5 | Blood and |
| | | | | | | Ocular tissues |
| 3 | 14 DB | Oral^{b} | 3 | 15 | 5 | Blood and |
| | | | | | | Ocular tissues |
| 4 | 15 DB | NA | NA | NA | NA | Blood and |
| | | | | | | Ocular tissues |
| 5 | 5 NZW | NA | NA | NA | NA | Blood and |
| | | | | | | Ocular tissues |

DB Dutch Belted.
NA Not applicable.
NZW New Zealand White.
Note:
Extra animals may be dosed for use as replacements in the event of a misdose or other unforeseen event, as applicable.
^{a}Single dose on Day 1.
^{b}Twice daily (BID) dose administered at 12-hour intervals for 14 days, with a single a.m. dose on Day 15. Each animal will receive 29 doses in total, and the doses will be administered at approximately the same time everyday +/−10 minutes.

For the pharmacokinetic determination, a single blood sample was taken from each rabbit (2 per time point) from Groups 1 and 2 at pre-dose, 1, 6, 24, 96, 168, and 240 hours post-dose on Day 1 and Group 3 at pre-dose. 1, 6, 24, 96, 168, and 240 hours post-dose on Day 15, with the eyes collected at the same time as blood sampling. A single blood sample was taken from Groups 4 and 5 at sacrifice. Plasma, vitreous humor (VH), retina, choroid-retina pigmented epithelium (Choroid-RPE), optic nerve (ON), and iris-ciliary body (I-CB) were analyzed by LC-MS/MS to quantify test article concentrations. The lower limit of quantitation (LLOQ) in plasma and vitreous humor was 1.0 ng/mL. The LLOQ was 6.0 ng/g in retina, Choroid-RPE, and I-CB and the optic nerve LLOQ was 15 ng/g, assuming tissue density of 1 g/mL where relevant. Mean pharmacokinetic parameters of Compound 1 following a 50 mg/kg twice daily (BID) oral dose to Dutch Belted rabbits for 14 consecutive days with a single morning dose on Day 15 are summarized in Table 5.

TABLE 5
Mean Pharmacokinetic Parameters of 15 mg/kg Compound
1 in New Zealand White and Dutch Belted Rabbit Plasma

| | Dose | Days of | Cₘₐₓ | Tₗₐₛₜ | AUCₗₐₛₜ | MRTₗₐₛₜ |
|---|---|---|---|---|---|---|
| Species | (mg/kg) | Dosing | (ng/mL) | (h) | (h*ng/mL) | (h) |
| New | 15 - single | 1 | 310 | 24 | 1430 | 4.01 |
| Zealand | dose | | | | | |
| White | | | | | | |
| Dutch | 15 - single | 1 | 318 | 24 | 1570 | 5.58 |
| Belted | dose | | | | | |
| Dutch | 15 BID | 15 | 845 | 96 | 3990 | 6.51 |
| Belted | | | | | | |

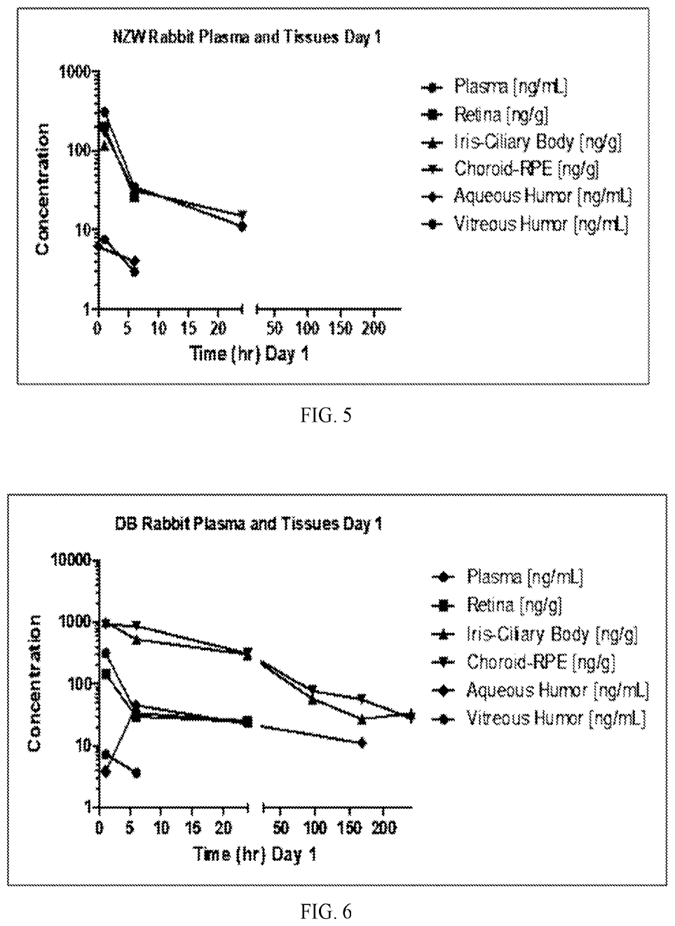

Following a single dose 15 mg/kg oral gavage of Compound 1 on Day 1, plasma exposure profiles were similar on Day 1 in NZW and DB rabbits, indicating that there is no exposure difference between the two strains. Retina concentrations were similar between NZW and DB rabbits on Day 1. DB rabbit concentrations were measurable through 24 hours post-dose on Day 1. Choroid-RPE concentrations were significantly higher in DB rabbit than in NZW rabbit on Day 1. Concentrations were measurable through 24 hours post-dose on Day 1 in both species. Iris-ciliary body concentrations were significantly higher in DB rabbit than in NZW rabbit on Day 1. Concentrations were measurable only at 1-hour post-dose on Day 1 in NZW rabbit, but through 240 hours post-dose on Day 1 for DB rabbit. Aqueous humor showed inconsistent and very low exposures across all three dose groups. Vitreous humor showed low exposures across all three dose groups. Mean plasma and tissue exposures in New Zealand White rabbit Day 1 (15 mg/kg single dose) are shown in Table 6 and graphically in FIG. 5.

TABLE 6
Mean Plasma and Tissue Concentrations of 15 mg/kg Compound 1 in New Zealand White Rabbit Day 1
Mean Plasma and Tissue Concentrations - New Zealand White Rabbit Day 1

| | | Retina | | Choroid-RPE | | Iris-Ciliary Body | | | Vitreous Humor | |
|---|---|---|---|---|---|---|---|---|---|---|
| | | | Retina | | Choroid- | | I-CB | | | Vitreous |
| | | | to | | RPE to | | to | Aqueous | | to |
| Time | Plasma | | Plasma | | Plasma | | Plasma | Humor | | Plasma |
| (hr) | [ng/mL] | [ng/g] | Ratio | [ng/g] | Ratio | [ng/g] | Ratio | [ng/mL] | [ng/mL] | Ratio |
| 0 | BLQ^{a} | BLQ^{a} | NC^{b} | BLQ^{a} | NC^{b} | BLQ^{a} | NC^{b} | 6.14 | BLQ^{a} | NCb |
| 1 | 310 | 200 | 0.65 | 172 | 0.56 | 117 | 0.38 | 4.00 | 7.61 | 0.02 |
| 6 | 35.1 | 26.0 | 0.74 | 31.5 | 0.90 | BLQ^{a} | NC^{b} | BLQ^{a} | 3.00 | 0.09 |
| 24 | 11.1 | BLQ^{a} | NC^{b} | 15.1 | 1.35 | BLQ^{a} | NC^{b} | BLQ^{a} | BLQ^{a} | NC^{b} |
| 96 | BLQ^{a} | BLQ^{a} | NC^{b} | BLQ^{a} | NC^{b} | BLQ^{a} | NC^{b} | BLQ^{a} | BLQ^{a} | NC^{b} |
| 168 | 500* | BLQ^{a} | NC^{b} | BLQ^{a} | NC^{b} | BLQ^{a} | NC^{b} | BLQ^{a} | BLQ^{a} | NC^{b} |
| 240 | BLQ^{a} | BLQ^{a} | NC^{b} | BLQ^{a} | NC^{b} | BLQ^{a} | NC^{b} | BLQ^{a} | BLQ^{a} | NC^{b} |

^{a}BLQ: Below Limit of Quantitation.
^{b}NC: Not Calculable

Mean plasma and tissue exposures in Dutch Belted rabbit Day 1 (15 mg/kg single dose) are shown in Table 7 and graphically in FIG. 6.

TABLE 7
Mean Plasma and Tissue Concentrations of 15 mg/kg Compound 1 in Dutch Belted Rabbit Day 1
Mean Plasma and Tissue Concentrations - Dutch Belted Rabbit Day 1

| | | Retina | | Choroid-RPE | | Iris-Ciliary Body | | | Vitreous Humor | |
|---|---|---|---|---|---|---|---|---|---|---|
| | | | Retina | | Choroid- | | I-CB | | | Vitreous |
| | | | to | | RPE to | | to | Aqueous | | to |
| Time | Plasma | | Plasma | | Plasma | | Plasma | Humor | | Plasma |
| (hr) | [ng/mL] | [ng/g] | Ratio | [ng/g] | Ratio | [ng/g] | Ratio | [ng/mL] | [ng/mL] | Ratio |
| 0 | BLQ^{a} | BLQa | NC^{b} | BLQ^{a} | NC^{b} | BLQ^{a} | NC^{b} | BLQ^{a} | BLQ^{a} | NC^{b} |
| 1 | 318 | 145 | 0.46 | 917 | 2.89 | 1010 | 3.18 | 3.80 | 7.26 | 0.02 |
| 6 | 33.7 | 29.6 | 0.88 | 866 | 25.7 | 528 | 15.7 | 45.3 | 3.66 | 0.11 |
| 24 | 25.4 | 24.7 | 0.97 | 311 | 12.3 | 300 | 11.8 | BLQ^{a} | BLQ^{a} | NC^{b} |
| 96 | BLQ^{a} | BLQ^{a} | NC^{b} | 77.5 | NC^{b} | 57.9 | NC^{b} | BLQ^{a} | BLQ^{a} | NC^{b} |
| 168 | BLQ^{a} | BLQ^{a} | NC^{b} | 56.3 | NC^{b} | 27.1 | NC^{b} | 11.1 | BLQ^{a} | NC^{b} |
| 240 | BLQ^{a} | BLQ^{a} | NC^{b} | 28.1 | NC^{b} | 33.6 | NC^{b} | BLQ^{a} | BLQ^{a} | NC^{b} |

^{a}BLQ: Below Limit of Quantitation.
^{b}NC: Not Calculable

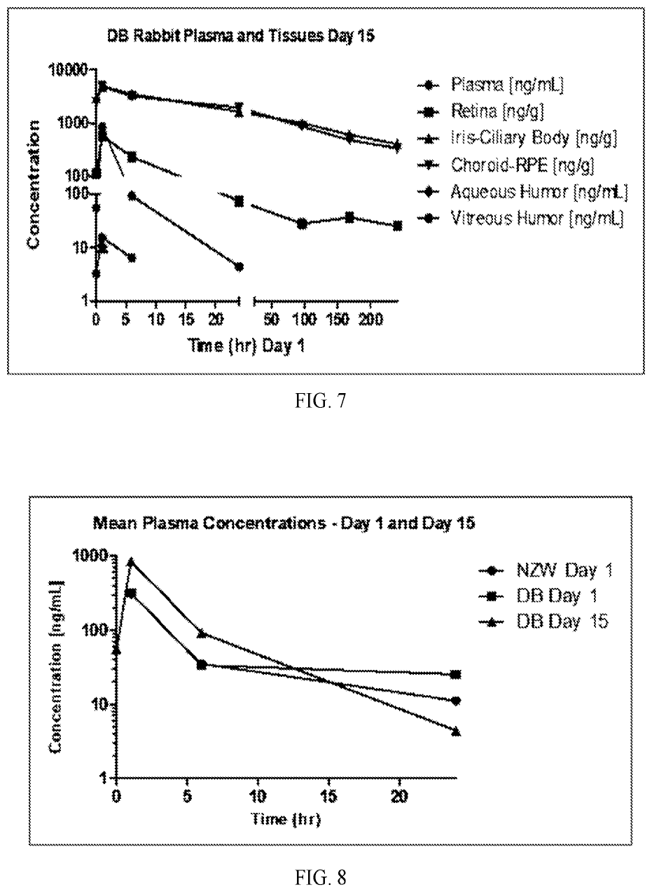

Following a twice daily (BID) 15 mg/kg oral gavage of Compound 1 for 14 days, favorable distribution to the Dutch Belted rabbit ocular tissues was achieved, though the replicate tissue concentrations at each timepoint within each ocular matrix were highly variable. The highest tissue levels were observed in the melanin-containing choroid-RPE. Concentrations in the vitreous humor were non-detectable (<1.0 ng/mL) after 24 hours post-dose. Approximately 2× accumulation was seen in plasma upon repeat dosing. Mean plasma and tissue exposure in Dutch Belted Rabbit on Day 15 (15 mg/kg BID) is shown in Table 8 and graphically in FIG. 7.

TABLE 8
Mean Plasma and Tissue Concentrations of 15 mg/kg Compound 1 in Dutch Belted Rabbit Day 15
Mean Plasma and Tissue Concentrations - Dutch Belted Rabbit Day 15

| | | Retina | | Choroid-RPE | | Iris-Ciliary Body | | | Vitreous Humor | |
|---|---|---|---|---|---|---|---|---|---|---|
| | | | Retina | | Choroid- | | I-CB | | | Vitreous |
| | | | to | | RPE to | | to | Aqueous | | to |
| Time | Plasma | | Plasma | | Plasma | | Plasma | Humor | | Plasma |
| (hr) | [ng/mL] | [ng/g] | Ratio | [ng/g] | Ratio | [ng/g] | Ratio | [ng/mL] | [ng/mL] | Ratio |
| 0 | 55.2 | 114 | 2.08 | 2460 | 44.6 | 2900 | 52.6 | BLQ^{a} | 3.27 | 0.03 |
| 1 | 845 | 576 | 0.68 | 4870 | 5.76 | 4920 | 5.82 | 10.6 | 15.4 | 0.03 |
| 6 | 92.2 | 237 | 2.57 | 3200 | 34.7 | 3510 | 38.1 | BLQ^{a} | 6.45 | 0.03 |
| 24 | 5.60 | 74.3 | 13.3 | 1960 | 350 | 1640 | 293 | BLQ^{a} | BLQ^{a} | NC^{b} |
| 96 | 3.19 | 28.1 | 8.79 | 866 | 271 | 1010 | 317 | BLQ^{a} | BLQ^{a} | NC^{b} |
| 168 | BLQ^{a} | 36.5 | NC^{b} | 485 | NC^{b} | 612 | NC^{b} | BLQ^{a} | BLQ^{a} | NC^{b} |
| 240 | BLQ^{a} | 25.3 | NC^{b} | 340 | NC^{b} | 408 | NC^{b} | BLQ^{a} | BLQ^{a} | NC^{b} |

^{a}BLQ: Below Limit of Quantitation.
^{b}NC: Not Calculable

Mean plasma concentrations were similar between New Zealand White (NZW) and Dutch Belted (DB) rabbits on Day 1, indicating similar plasma exposure profiles between the two strains. Approximately 2× accumulation was seen on Day 15 upon repeat dosing. Mean plasma concentrations across dose groups on Day 1 and Day 15 are shown in Table 9 and graphically in FIG. 8.

TABLE 9
Mean Plasma Concentrations of 15 mg/kg Compound
1 in New Zealand White Rabbit Day 1 and
Dutch Belted Rabbit on Day 1 and Day 15

| | Mean Plasma Concentrations [ng/mL] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | BLQ^{a} | BLQ^{a} | 55.2 |
| 1 | 310 | 318 | 845 |
| 6 | 35.1 | 33.7 | 92.2 |
| 24 | 11.1 | 25.4 | 5.60 |
| 96 | BLQ^{a} | BLQ^{a} | 3.19 |
| 168 | 500* | BLQ^{a} | BLQ^{a} |
| 240 | BLQ^{a} | BLQ^{a} | BLQ^{a} |

^{a}BLQ: Below Limit of Quantitation.
^{b}NC: Not Calculable
*Outlier Value: Not used in PK calculations.
LLOQ: 2.5 ng/mL

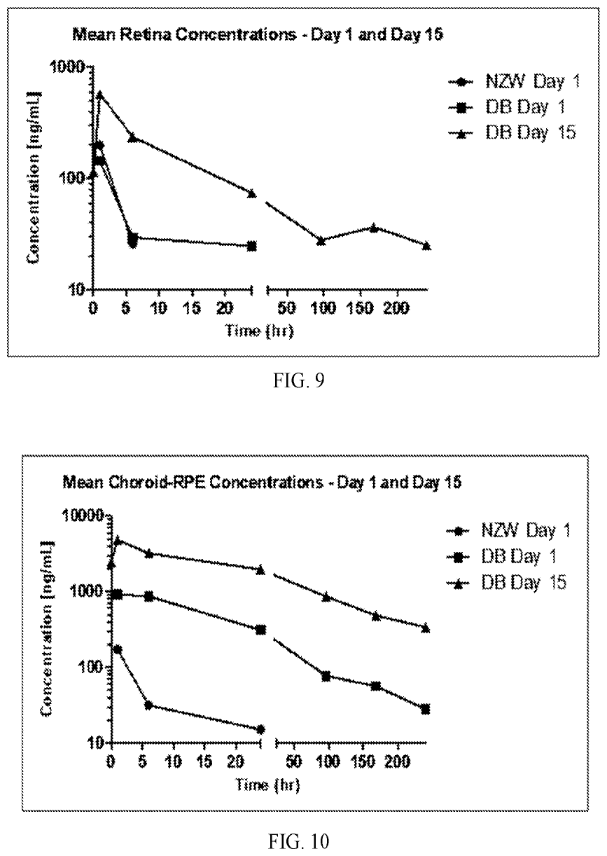

Retina concentrations were similar between NZW and DB Rabbit on Day 1. Retina exposures were significantly higher in DB rabbit on Day 15 than on Day 1. DB rabbit concentrations were measurable through 24 hours post-dose on Day 1 and through 240 hours post-dose on Day 15. Mean retina concentrations on Day 1 and Day 15 are shown in Table 10 and graphically in FIG. 9.

TABLE 10
Mean Retina Concentrations of 15 mg/kg Compound
1 in New Zealand White Rabbit on Day 1 and
Dutch Belted Rabbit on Day 1 and Day 15

| | Mean Retina Concentrations [ng/g] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | BLQ^{a} | BLQ^{a} | 114 |
| 1 | 200 | 145 | 576 |
| 6 | 26.0 | 30 | 237 |
| 24 | BLQ^{a} | 25 | 74.3 |
| 96 | BLQ^{a} | BLQ^{a} | 28.1 |
| 168 | BLQ^{a} | BLQ^{a} | 36.5 |
| 240 | BLQ^{a} | BLQ^{a} | 25.3 |

^{a}BLQ: Below Limit of Quantitation.
LLOQ: 15 ng/g

Mean Choroid-RPE concentrations were significantly higher in DB rabbit than NZW rabbit on Day 1. Choroid-RPE exposures were significantly higher in DB rabbit on Day 15 than on Day 1. Concentrations were measurable through 24 hours post-dose on Day 1 in NZW rabbit and through 240 hours post-dose on Day 1 and Day 15 for DB Rabbit. Mean choroid-RPE concentrations on Day 1 and Day 15 (15 mg/kg) are shown in Table 11 and graphically in FIG. 10.

TABLE 11
Mean Choroid-RPE Concentrations of 15 mg/kg Compound
1 in New Zealand White Rabbit on Day 1 and Dutch
Belted Rabbit on Day 1 and Day 15

| | Mean Choroid-RPE Concentrations [ng/g] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | BLQ^{a} | BLQ^{a} | 2460 |
| 1 | 172 | 917 | 4870 |
| 6 | 31.5 | 866 | 3200 |
| 24 | 15.1 | 311 | 1960 |
| 96 | BLQ^{a} | 77.5 | 866 |
| 168 | BLQ^{a} | 56.3 | 485 |
| 240 | BLQ^{a} | 28.1 | 340 |

^{a}BLQ: Below Limit of Quantitation.
LLOQ: 15 ng/g

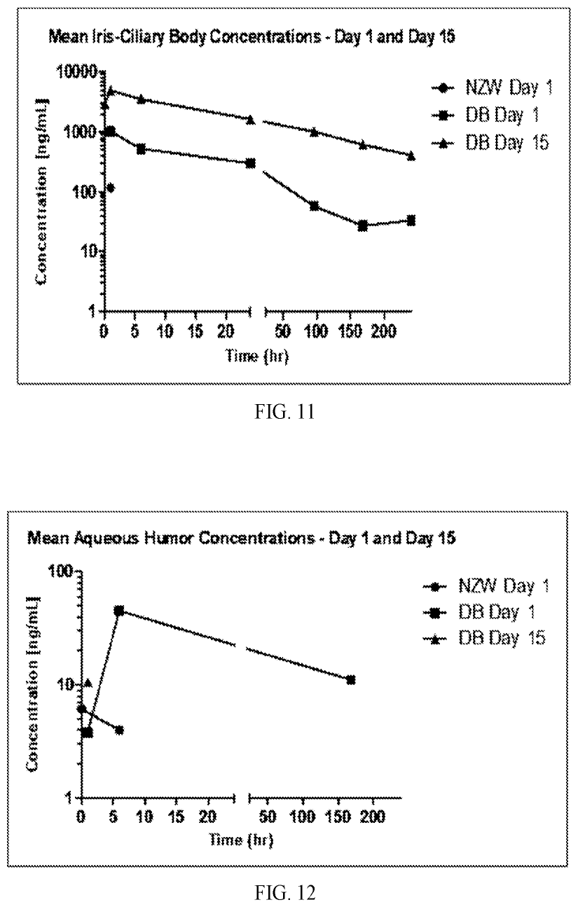

Iris-ciliary body concentrations were significantly higher in DB rabbit than in NZW rabbit. Iris-ciliary body exposures were significantly higher in DB rabbit on Day 15 than on Day 1. Concentrations were measurable only at 1-hour post-dose in NZW rabbit on Day 1 but through 240 hours post-dose on Day 1 and Day 15 for DB Rabbit. Mean iris-ciliary body concentrations on Day 1 and Day 15 are shown in Table 12, and graphically in FIG. 11.

TABLE 12
Mean Iris-Ciliary Body Concentrations of 15 mg/kg
Compound 1 in New Zealand White Rabbit on Day
1 and Dutch Belted Rabbit on Day 1 and Day 15

| | Mean Iris-Ciliary Body Concentrations [ng/g] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | BLQ^{a} | BLQ^{a} | 2900 |
| 1 | 117 | 1010 | 4920 |
| 6 | BLQ^{a} | 528 | 3510 |
| 24 | BLQ^{a} | 300 | 1640 |
| 96 | BLQ^{a} | 57.9 | 1010 |
| 168 | BLQ^{a} | 27.1 | 612 |
| 240 | BLQ^{a} | 33.6 | 408 |

^{a}BLQ: Below Limit of Quantitation
LLOQ: 15 ng/g

Aqueous humor showed inconsistent and very low exposures across all three dose groups. Aqueous humor concentrations on Day 1 and Day 15 are shown on Table 13, and graphically in FIG. 12.

TABLE 13
Mean Aqueous Humor Concentrations of 15 mg/kg
Compound 1 in New Zealand White Rabbit on Day
1 and Dutch Belted Rabbit on Day 1 and Day 15

| | Mean Aqueous Humor Concentrations [ng/mL] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | 6.14 | BLQ^{a} | BLQ^{a} |
| 1 | 4.00 | 3.80 | 10.6 |
| 6 | BLQ^{a} | 45.3 | BLQ^{a} |
| 24 | BLQ^{a} | BLQ^{a} | BLQ^{a} |
| 96 | BLQ^{a} | BLQ^{a} | BLQ^{a} |
| 168 | BLQ^{a} | 11.1 | BLQ^{a} |
| 240 | BLQ^{a} | BLQ^{a} | BLQ^{a} |

^{a}BLQ: Below Limit of Quantitation.
LLOQ: 2.5 ng/mL

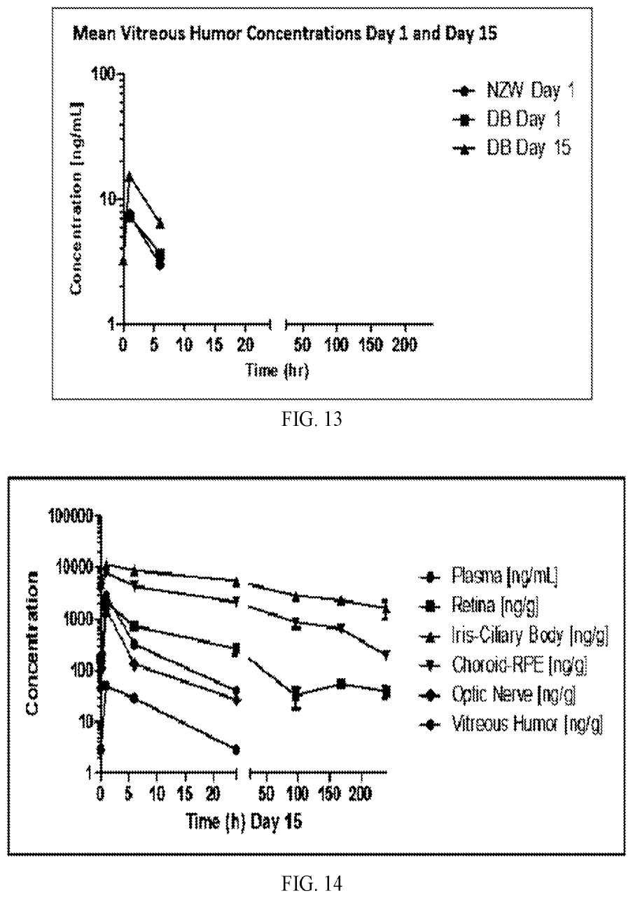

Vitreous humor showed low exposures across all three dose groups with minor accumulation over 15 days of dosing. Mean Vitreous humor concentrations on Day 1 and Day 15 are shown in Table 14 and graphically in FIG. 13.

TABLE 14
Mean Vitreous Humor Concentrations of 15 mg/kg
Compound 1 in New Zealand White Rabbit on Day
1 and Dutch Belted Rabbit on Day 1 and Day 15

| | Mean Vitreous Humor Concentrations [ng/mL] | | |
|---|---|---|---|
| | New Zealand White | Dutch Belted | Dutch Belted |
| | Day 1 (15 mg/kg | Day 1 (15 mg/kg | Day 15 (15 mg/kg |
| Time (hr) | single dose) | single dose) | BID) |
| 0 | BLQ^{a} | BLQ^{a} | 3.27 |
| 1 | 7.61 | 7.26 | 15.4 |
| 6 | 3.00 | 3.66 | 6.45 |
| 24 | BLQ^{a} | BLQ^{a} | BLQ^{a} |
| 96 | BLQ^{a} | BLQ^{a} | BLQ^{a} |
| 168 | BLQ^{a} | BLQ^{a} | BLQ^{a} |
| 240 | BLQ^{a} | BLQ^{a} | BLQ^{a} |

^{a}BLQ: Below Limit of Quantitation.
LLOQ: 2.5 ng/mL

### Example 4. Ocular Tissue Distribution of Compound 1 Following 50 mg/kg Oral Administration in Rabbits

This study was conducted to evaluate the ocular tissue and plasma pharmacokinetics, ocular tissue distribution, and tolerability of Compound 1 following a twice daily (BID) 50 mg/kg oral gavage in Dutch Belted and New Zealand White rabbits. Study design, including group designation and dose levels, are outlined in Table 15. Fourteen Dutch Belted rabbits and three New Zealand White rabbits were dosed for 14 consecutive days BID, with a single morning dose of Compound 1 on Day 15. Three Dutch Belted rabbits were dosed for 7 days BID with a single morning dose of compound 1 on Day 8 to determine if steady state plasma and tissue concentrations had been reached by Day 8. Two Dutch Belted rabbits received vehicle only oral gavage doses for tolerability assessment. Compound 1 was formulated as a suspension in 0.5% hydroxypropyl methylcellulose (HPMC) with 0.1% Tween 80. The dose volume was 5 mL/kg/dose for all groups. Safety was assessed with toxicological and ophthalmic examinations, including slit lamp biomicroscopy, indirect ophthalmoscopy, rebound tonometry, and ultrasonic corneal pachymetry.

TABLE 15
Group Designations and Dose Levels
EXPERIMENTAL DESIGN
Study Design
Group Designations and Dose Levels

| | | | | | Target Dose | Target | |
|---|---|---|---|---|---|---|---|
| | Number | | | Dose | Level | Dose | |
| | of Male | Test | Dose | Concentration | (mg/kg/dose)/ | Volume | Sample |
| Group | Animals | Article | Route | (mg/mL) | (mg/kg/day) | (mL/kg/dose) | Collection |
| 1 | 2 DB | Vehicle | Oral^{a} | NA | NA/NA | 5 | Blood and |
| | | | | | | | Ocular tissues |
| 2 | 3 DB | ACH- | Oral^{b} | 10 | 50/100 | 5 | Blood and |
| | | 4471 | | | | | Ocular tissues |
| 3 | 14 DB | ACH- | Oral^{a} | 10 | 50/100 | 5 | Blood and |
| | | 4471 | | | | | Ocular tissues |
| 4 | 3 NZW | ACH- | Oral^{a} | 10 | 50/100 | 5 | Blood and |
| | | 4471 | | | | | Ocular tissues |
| 5 | 5 DB | NA | NA | NA | NA/NA | NA | Blood and |
| | | | | | | | Ocular tissues |

DB Dutch Belted.
NA Not applicable.
NZW New Zealand White.
Note:
Extra animals may be dosed for use as replacements in the event of a misdose or other unforeseen event, as applicable.
^{a}Animals will receive a twice daily (BID) dose (approximately 12 hours apart) for 14 days, with a single a.m. dose on Study Day 15 (for a total of 29 doses).
^{b}Animals will receive a twice daily (BID) dose (approximately 12 hours apart) for 7 days, with a single a.m. dose on Study Day 8 (for a total of 15 doses).

For the pharmacokinetic determination, a single blood sample was taken from each rabbit from Groups 1 and 4 at one hour post-dose on Day 15, Group 2 at one hour post-dose on Day 8, and Group 3 at pre-dose, 1, 6, 24, 96, 168, and 240 hours post-dose on Day 15, with the eyes collected at the same time as blood sampling. Plasma, vitreous humor (VH), retina, choroid-retina pigmented epithelium (Choroid-RPE), optic nerve (ON), and iris-ciliary body (I-CB) were analyzed by LC-MS/MS to quantify test article concentrations. The lower limit of quantitation (LLOQ) in plasma and vitreous humor was 1.0 ng/mL. The LLOQ was 6.0 ng/g in retina, choroid-RPE, and I-CB and the optic nerve LLOQ was 15 ng/g, assuming tissue density of 1 g/mL where relevant.

Following a twice daily (BID) 50 mg/kg oral gavage of Compound 1 for 14 days, favorable distribution to the Dutch Belted rabbit ocular tissues was achieved, though the replicate tissue concentrations at each timepoint within each ocular matrix were highly variable. The highest tissue levels were observed in the melanin containing choroid-RPE. Concentrations in plasma and vitreous humor were non-detectable (<1.0 ng/mL) after 24 hours post-dose. Concentrations in optic nerve were non-detectable (<1.0 ng/mL) after 24 hours post-dose. Mean pharmacokinetic parameters of Compound 1 following a 50 mg/kg BID oral dose to Dutch Belted rabbits for 14 consecutive days with a single morning dose on Day 15 are summarized in Table 16.

TABLE 16
Mean Pharmacokinetic Parameters of Compound 1 in Dutch Belted
Rabbit Plasma and Ocular Tissue Following 50 mg/kg (BID) Oral
Dosing for 14 Consecutive Days with a Single Morning Dose

| | T_{1/2} | Tₘₐₓ | Cₘₐₓ | AUC₀₋ₜ | AUC_{0-∞} | MRTₗₐₛₜ |
|---|---|---|---|---|---|---|
| Eye Tissue | (h) | (h) | (ng/g) | (h*ng/g) | (h*ng/g) | (h) |
| Choroid- | 66.4 | 1 | 8020 | 274000 | 292000 | 68.6 |
| RPE^{a} | | | | | | |
| Iris-Ciliary | 178 | 1 | 11500 | 801000 | 1220000 | 88.0 |
| Body^{a} | | | | | | |
| Optic | 7.82 | 1 | 1550 | 4880 | 5180 | 4.79 |
| Nerve^{a} | | | | | | |
| Plasma^{b} | 5.98 | 1 | 2900 | 9790 | 10100 | 4.73 |
| Retina^{c} | 96.1 | 1 | 2120 | 30300 | 35700 | 53.2 |
| Vitreous | 5.44 | 1 | 49.2 | 419 | 442 | 7.27 |
| Humor^{a} | | | | | | |

The plasma AUC_{0-∞} was 10100 h*ng/g with the mean MRTₗₐₛₜ being 4.73 h. The iris-ciliary body AUC_{0-∞} was 1220000 h*ng/g (120-fold higher than plasma) and the mean plasma MRTₗₐₛₜ was 88.0 h (19-fold longer than plasma). The choroid-RPE AUC_{0-∞} was 292000 h*ng/g (four-fold higher than plasma) with the mean MRTₗₐₛₜ being 68.6 h (15-fold longer than plasma). The retina AUC_{0-∞} was 35700 h*ng/g (4-fold higher than plasma) and the mean retina MRTₗₐₛₜ was 53.2 h (11-fold longer than plasma). The extended residence time in the melanin containing iris-ciliary body, choroid-RPE, and retina suggest that these tissues may act as a depot for Compound 1 disposition.

The plasma exposure of Compound 1 in DB rabbit at one-hour post-dose on Day 8 (Group 2) was similar to that in DB rabbit at one-hour post-dose on Day 15 (Group 3) indicating that steady state systemic exposure was reached by Day 8. Additionally, plasma exposure in NZW rabbit at one-hour post-dose on Day 15 (Group 4) was similar to DB rabbit at Day 8 and Day 15, demonstrating no significant difference in plasma exposure between the DB and NZW rabbit strains. Likewise, for retina, optic nerve, and vitreous humor, similar tissue concentrations were observed between DB rabbit on Day 8 and Day 15 as well as NZW rabbit on Day 15. Choroid-RPE tissue exposure in DB rabbit were similar between Day 8 and Day 15 at one-hour post-dose, while choroid-RPE tissue exposures in NZW were approximately five-fold lower at one-hour post-dose on Day 15. Finally, iris-ciliary body tissue exposure in DB rabbit was similar between Day 8 and Day 15 at one-hour post-dose, while iris-ciliary body tissue exposures in NZW were approximately 14-fold lower at one-hour post-dose on Day 15. The extended residence time of Compound 1 in melanin containing tissues versus plasma and non-melanin containing tissues in DB rabbits as well as the lower concentrations in choroid-RPE and I-CB in the melanin depleted NZW rabbits suggest that Compound 1 binds to melanin in situ and that melanin may act as a reservoir for Compound 1.

Mean concentration in plasma and ocular matrices of Compound 1 are presented in Table 17. Mean (SD) concentrations of Compound 1 following a 50 mg/kg BID oral gavage dose (Day 15) to DB rabbits are graphically represented in FIG. 14.

TABLE 17
Plasma and Tissue Summary of 50 mg/kg Compound 1 Exposure - Day 15
Compound 1 Exposure Dutch Belted Rabbit

| | | | | | | | | | Optic | | |
|---|---|---|---|---|---|---|---|---|---|---|---|
| | | | Retina: | Choroid- | Chr-RPE: | | I-CB: | Optic | Nerve: | | Vitreous: |
| Time (h) | Plasma | Retina | Plasma | RPE | Plasma | I-CB | Plasma | Nerve | Plasma | Vitreous | Plasma |
| Day 15 | [ng/mL] | [ng/g] | Ratio | [ng/g] | Ratio | [ng/g] | Ratio | [ng/g] | Ratio | [ng/g] | Ratio |
| 0 | 104 | 159 | 1.53 | 3920 | 37.7 | 5080 | 48.8 | 45.3 | 0.298 | 2.91 | 0.0339 |
| 1 | 2900 | 2120 | 0.731 | 8020 | 2.77 | 11500 | 3.97 | 1550 | 0.439 | 49.2 | 0.0170 |
| 6 | 320 | 725 | 2.27 | 4370 | 13.7 | 8820 | 27.6 | 133 | 0.414 | 28.7 | 0.0880 |
| 24 | 39.9 | 265 | 6.65 | 2100 | 52.7 | 5560 | 140 | 27.0 | 0.705 | 2.90 | 0.0725 |
| 96 | BLQ^{a} | 32.3 | NA | 873 | NA | 2840 | NA | BLQ^{b} | NA | BLQ^{c} | NA |
| 168 | BLQ^{a} | 54.9 | NA | 645 | NA | 2290 | NA | BLQ^{b} | NA | BLQ^{c} | NA |
| 240 | BLQ^{a} | 39.3 | NA | 194 | NA | 1620 | NA | BLQ^{b} | NA | BLQ^{c} | NA |

^{a}BLQ: LLOQ for Plasma, 1 ng/mL.
^{b}BLQ: LLOQ for Optic Nerve, 15 ng/g.
^{c}BLQ: LLOQ for Vitreous Humor, 1 ng/g.

Retina concentrations have a similar exposure profile as plasma through 24 hours post-dose on Day 15, and experience prolonged exposure out to 240 hours post-dose. Choroid-RPE and iris-ciliary body have significantly higher exposures than plasma and retina. The iris-ciliary body experiences significantly higher exposure than the Choroid-RPE tissues. Optic Nerve has a similar exposure profiles to plasma out to 24 hours post-dose. Vitreous humor has a significantly lower exposure profile than plasma out to 24 hours post-dose.

### Example 5. Combined Results of Plasma and Ocular Tissue Pharmacokinetics in New Zealand White and Dutch Belted Rabbits Receiving Compound 1

Two pharmacokinetic studies were completed to investigate the pharmacokinetics of Compound 1 in rabbit eyes after single and multiple oral administrations. In the first study, 14 male Dutch Belted rabbits received oral Compound 1 or vehicle at doses up to 50 mg/kg twice-daily (BID) for 14 days and one dose at day 15. In the second study, 14 New Zealand White rabbits (Group 1) received oral Compound 1 or vehicle at a single dose of 15 mg/kg, 14 Dutch Belted Rabbits received oral Compound 1 or vehicle at a single dose of 15 mg/kg, and 14 Dutch Belted Rabbits received oral Compound 1 or vehicle at doses up to 15 mg/kg twice-daily (BID) for 14 days and one dose at day 15. Study design including group designation and dose levels are outlined in Table 18.

TABLE 18
Group Designations and Dose Information

| | Number of | Dose | Dose | Dose | |
|---|---|---|---|---|---|
| Group | Male Animals | Route | Level | Regimen | Collections |
| 1 | 14 NZW | Oral | 15 mg/kg | Single Dose | Plasma and Ocular |
| (Study 2) | | | | | Tissues |
| 2 | 14 DB | Oral | 15 mg/kg | Single Dose | Plasma and Ocular |
| (Study 2) | | | | | Tissues |
| 3 | 14 DB | Oral | 15 mg/kg | BID × 14 Days & | Plasma and Ocular |
| (Study 2) | | | | One Dose at Day 15 | Tissues after |
| | | | | | last dose |
| 4 | 14 DB | Oral | 50 mg/kg | BID × 14 Days & | Plasma and Ocular |
| (Study 1) | | | | One Dose at Day 15 | Tissues after |
| | | | | | last dose |

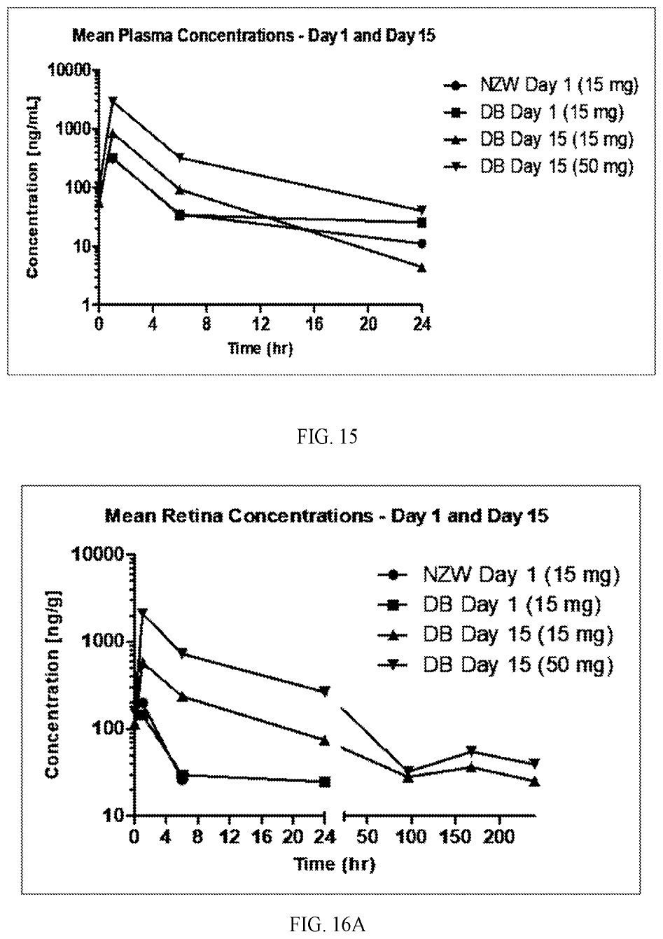

Mean plasma concentrations were determined by liquid chromatography/mass spectrometry (LC-MS/MS) on Day 1 (Groups 1 and 2) and Day 15 (Groups 3 and 4). As shown in Tables 19 and 20, and graphically in FIG. 15, plasma pharmacokinetic profiles and concentrations are similar between New Zealand White (NZW) rabbits (Group 1) and Dutch Belted (DB) rabbits (Group 2) on Day 1. Only minor accumulation was seen after repeat dosing at 15 mg/kg BID in DB rabbits (Group 3). Exposures are largely dose-proportional from 15 to 50 mg/kg BID in DB rabbits.

TABLE 19
Pharmacokinetic measures in Plasma from New
Zealand White and Dutch Belted Rabbits

| | | | | Cₘₐₓ | | AUCₗₐₛₜ | |
|---|---|---|---|---|---|---|---|
| | Dose | | Days of | (ng/ | Tₗₐₛₜ | (h*ng/ | MRTₗₐₛₜ |
| Species | (mg/kg) | | Dosing | mL) | (h) | mL) | (h) |
| New | 15 | single dose | 1 | 310 | 24 | 1430 | 4.01 |
| Zealand | | | | | | | |
| White | | | | | | | |
| Dutch | 15 | single dose | 1 | 318 | 24 | 1570 | 5.58 |
| Belted | | | | | | | |
| Dutch | 15 | BID | 15 | 845 | 96 | 3990 | 6.51 |
| Belted | | | | | | | |
| Dutch | 50 | BID | 15 | 2900 | 24 | 9790 | 4.73 |
| Belted | | | | | | | |

TABLE 20
Mean Plasma Concentrations from New Zealand
White and Dutch Belted Rabbits

| | Mean Plasma Concentration [ng/mL] | | | |
|---|---|---|---|---|
| | NZW Day 1 | DB Day 1 | DB Day 15 | DB Day 15 |
| | 15 mg/kg | 15 mg/kg | 15 mg/kg | 50 mg/kg |
| Time (hr) | single dose | single dose | BID | BID |
| 0 | BLQ | BLQ^{a} | 55.2 | 104 ± 68 |
| 1 | 310 | 318 | 845 | 2895 ± 884 |
| 6 | 35.1 | 33.7 | 92.2 | 321 ± 70.0 |
| 24 | 11.1 | 25.4 | 5.6 | 39.9 ± 4.45 |
| 96 | BLQ | BLQ^{a} | 3.19 | BLQ |
| 168 | 500* | BLQ^{a} | BLQ^{a} | BLQ |
| 240 | BLQ | BLQ^{a} | BLQ^{a} | BLQ |

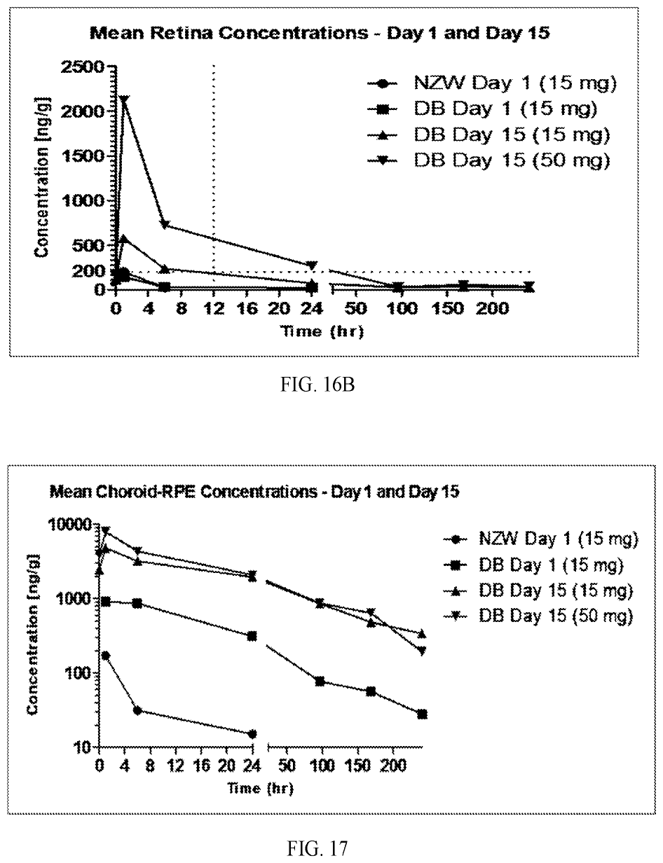

Cₘₐₓ in retina is not higher in DB than in NZW. MRT is much longer in DB than in NZW, which shows that the prolonged exposure in DB retina is from the drug depot. Significant accumulation of drug was seen after repeat dosing in DB, most prominent in AUC from Day 1 to Day 15. This is consistent with the slow clearance from the tissue as a consequence of replenishment of the drug from Choroid-RPE. In DB, AUC is less than dose proportional from 15 to 50 mg BID although Cₘₐₓ is constantly ˜0.7 fold over the plasma level. This supports the notion of saturation of the drug depot. Table 21 shows the retina pharmacokinetic parameters. Table 22 shows the mean retina concentrations from New Zealand White and Dutch Belted rabbits and graphically in FIGS. 16A and 16B.

TABLE 21
Mean Retina Pharmacokinetic Parameters of Compound
1 in New Zealand White and Dutch Belted Rabbits

| | Dose | | Days of | T_{1/2} | Cₘₐₓ | Tₗₐₛₜ | AUCₗₐₛₜ | MRTₗₐₛₜ | MRT_{Inf} |
|---|---|---|---|---|---|---|---|---|---|
| Species | (mg/kg) | | Dosing | (h) | (ng/m) | (h) | (h*ng/mL) | (h) | (h) |
| New | 15 | single dose | 1 | NC^{a} | 200 | 6 | 665 | 1.49 | NC^{a} |
| Zealand | | | | | | | | | |
| White | | | | | | | | | |
| Dutch | 15 | single dose | 1 | NC^{a} | 145 | 24 | 998 | 7.83 | NC^{a} |
| Belted | | | | | | | | | |
| Dutch | 15 | BID | 15 | NC^{a} | 576 | 240 | 13400 | 71.0 | NC^{a} |
| Belted | | | | | | | | | |
| Dutch | 50 | BID | 15 | 96.1 | 2120 | 240 | 30300 | 53.2 | NC^{a} |
| Belted | | | | | | | | | |

^{a}NC: Not Calculated

TABLE 22
Mean Retina Concentrations of Compound 1 in New Zealand White and Dutch Belted Rabbits

| | Mean Retina Concentration | | | | | | | |
|---|---|---|---|---|---|---|---|---|
| | NZW Day 1 | | DB Day 1 | | DB Day 15 | | DB Day 15 | |
| | 15 mg/kg single dose | | 15 mg/kg single dose | | 15 mg/kg BID | | 50 mg/kg BID | |
| | | vs. | | vs. | | vs. | | vs. |
| Time (hr) | ng/g | Plasma | ng/g | Plasma | ng/g | Plasma | ng/g | Plasma |
| 0 | BLQ | NC | BLQ | NC | 114 | 2.08 | 159 ± 87.9 | 1.53 |
| 1 | 200 | 0.65 | 145 | 0.46 | 576 | 0.68 | 2122 ± 376 | 0.73 |
| 6 | 26 | 0.74 | 30 | 0.88 | 237 | 2.57 | 725 ± 117 | 2.26 |
| 24 | BLQ | NC | 25 | 0.97 | 74.3 | 13.3 | 265 ± 113 | 6.65 |
| 96 | BLQ | NC | BLQ | NC | 28.1 | 8.79 | 32.3 ± 24.2 | NC |
| 168 | BLQ | NC | BLQ | NC | 36.5 | NC | 54.9 ± 7.28 | NC |
| 240 | BLQ | NC | BLQ | NC | 25.3 | NC | 39.4 ± 20.1 | NC |

BLQ: Below Limit of Quantitation;
NC, not calculated

In Choroid-RPE, exposures are much higher in DB than in NZW at Day 1 (higher Cₘₐₓ, AUC, and MRT), which confirms that Compound 1 binds to melanin, leading to much slower clearance from tissue. Significant accumulation of the drug was seen after repeat dosing in DB (˜6-fold), which is consistent with a long MRT. In DB at Day 15, AUC is similar between 15 mg and 50 mg BID groups, whereas Cₘₐₓ is less than dose proportional, which indicates saturation of the drug depot. Table 23 shows the Choroid-RPE pharmacokinetic parameters. Table 24 shows the mean Choroid-RPE concentrations from New Zealand White and Dutch Belted rabbits and graphically in FIG. 17.

TABLE 23
Mean Choroid-RPE Pharmacokinetic Parameters of Compound
1 in New Zealand White and Dutch Belted Rabbits

| | | | Days of | T_{1/2} | Cₘₐₓ | Tₗₐₛₜ | AUCₗₐₛₜ | MRTₗₐₛₜ | MRT_{Inf} |
|---|---|---|---|---|---|---|---|---|---|
| Species | Dose (mg/kg) | | Dosing | (h) | (ng/mL) | (h) | (h*ng/mL) | (h) | (h) |
| New Zealand | 15 | single dose | 1 | NC | 172 | 24 | 1010 | 5.87 | NC |
| White | | | | | | | | | |
| Dutch Belted | 15 | single dose | 1 | 98.4 | 917 | 240 | 37400 | 49.7 | 81.8 |
| Dutch Belted | 15 | BID | 15 | 107 | 4870 | 240 | 250000 | 68.5 | 125 |
| Dutch Belted | 50 | BID | 15 | 66.4 | 8020 | 240 | 274000 | 68.60 | NC^{a} |

^{a}NC: Not Calculated

TABLE 24
Mean Choroid-RPE Concentrations of Compound 1
in New Zealand White and Dutch Belted Rabbits

| | Mean Choroid-RPE Concentration | | | | | | | |
|---|---|---|---|---|---|---|---|---|
| | NZW Day 1 | | DB Day 1 | | DB Day 15 | | DB Day 15 | |
| | 15 mg/kg single dose | | 15 mg/kg single dose | | 15 mg/kg BID | | 50 mg/kg BID | |
| | | vs. | | vs. | | vs. | | vs. |
| Time (hr) | ng/g | Plasma | ng/g | Plasma | ng/g | Plasma | ng/g | Plasma |
| 0 | BLQ | NC | BLQ | NC | 2460 | 44.6 | 3918 ± 950 | 37.6 |
| 1 | 172 | 0.56 | 917 | 2.89 | 4870 | 5.76 | 8025 ± 2350 | 2.77 |
| 6 | 31.5 | 0.9 | 866 | 25.7 | 3200 | 34.7 | 4373 ± 1586 | 13.6 |
| 24 | 15.1 | 1.35 | 311 | 12.3 | 1960 | 350 | 2097 ± 273 | 52.6 |
| 96 | BLQ | NC | 77.5 | NC | 866 | 271 | 873 ± 392 | NC |
| 168 | BLQ | NC | 56.3 | NC | 485 | NC | 645 ± 67.2 | NC |
| 240 | BLQ | NC | 28.1 | NC | 340 | NC | 195 ± 41.5 | NC |

BLQ: Below Limit of Quantitation;
NC, not calculated

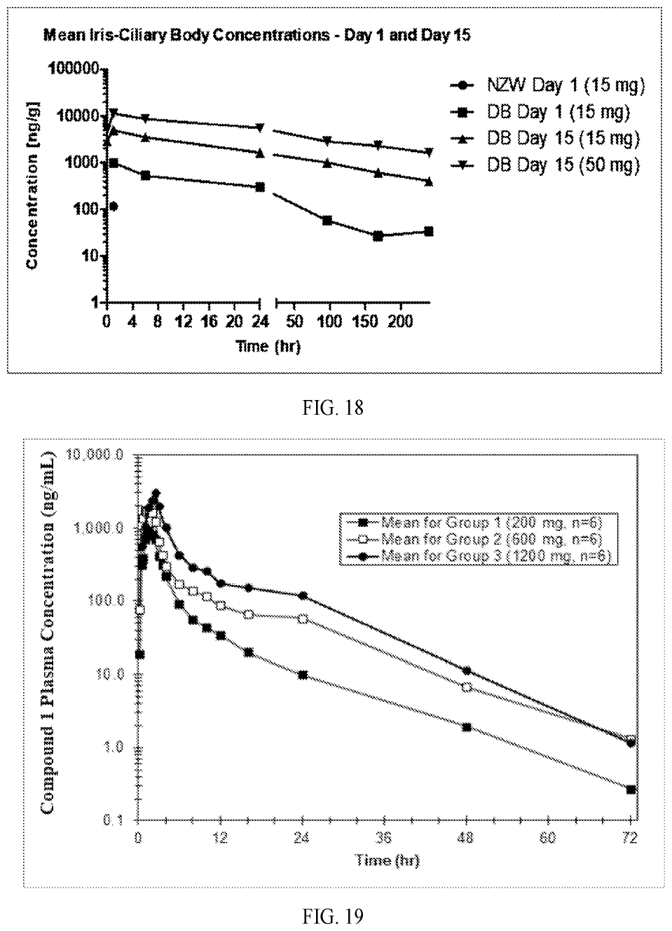

In the iris-ciliary body, exposures are much higher in DB than in NZW at Day 1 (higher Cₘₐₓ, AUC, and MRT), which confirms that Compound 1 binds to melanin, leading to much slower clearance from tissue. Significant accumulation of the drug was seen after repeat dosing in DB, which is consistent with a long MRT. In DB at Day 15, AUC is similar between 15 mg and 50 mg BID groups whereas Cₘₐₓ is less than dose proportional, which is an indication of the saturation of the drug depot. Table 25 shows the iris-ciliary body pharmacokinetic parameters. Table 26 shows the mean iris-ciliary body concentrations from New Zealand White and Dutch Belted rabbits and graphically in FIG. 18.

TABLE 25
Mean Iris-Ciliary Body Pharmacokinetic Parameters of Compound
1 in New Zealand White and Dutch Belted Rabbits

| | | | Days of | T_{1/2} | Cₘₐₓ | Tₗₐₛₜ | AUCₗₐₛₜ | MRTₗₐₛₜ | MRT_{Inf} |
|---|---|---|---|---|---|---|---|---|---|
| Species | Dose (mg/kg) | | Dosing | (h) | (ng/mL) | (h) | (h*ng/mL) | (h) | (h) |
| New Zealand | 15 | single dose | 1 | NC^{a} | 117 | 1 | 58.5 | 1.0 | NC^{a} |
| White | | | | | | | | | |
| Dutch Belted | 15 | single dose | 1 | 56.0 | 1010 | 240 | 29900 | 46.2 | 69.0 |
| Dutch Belted | 15 | BID | 15 | 107 | 4920 | 240 | 261000 | 76.2 | 81.5 |
| Dutch Belted | 50 | BID | 15 | 178 | 11500 | 240 | 274000 | 88.0 | 138 |

^{a}NC: Not Calculated

TABLE 26
Mean Iris-Ciliary Body Concentrations of Compound
1 in New Zealand White and Dutch Belted Rabbits

| | Mean Iris-Ciliary Body Concentration | | | | | | | |
|---|---|---|---|---|---|---|---|---|
| | NZW Day 1 | | DB Day 1 | | DB Day 15 | | DB Day 15 | |
| | 15 mg/kg single dose | | 15 mg/kg single dose | | 15 mg/kg BID | | 50 mg/kg BID | |
| | | vs. | | vs. | | vs. | | vs. |
| Time (hr) | ng/g | Plasma | ng/g | Plasma | ng/g | Plasma | ng/g | Plasma |
| 0 | BLQ | NC | BLQ | NC | 2900 | 52.6 | 5076 ± 586 | 48.7 |
| 1 | 117 | 0.38 | 1010 | 3.18 | 4920 | 5.82 | 11475 ± 2022 | 3.96 |
| 6 | BLQ | NC | 528 | 15.7 | 3510 | 38.1 | 8822 ± 2621 | 27.5 |
| 24 | BLQ | NC | 300 | 11.8 | 1640 | 293 | 5556 ± 2128 | 139 |
| 96 | BLQ | NC | 57.9 | NC | 1010 | 317 | 2837 ± 1136 | NC |
| 168 | BLQ | NC | 27.1 | NC | 612 | NC^{b} | 2292 ± 445 | NC |
| 240 | BLQ | NC | 33.6 | NC | 408 | NC^{b} | 1624 ± 1275 | NC |

BLQ: Below Limit of Quantitation;
NC, not calculated

In Vitreous Humor, similar exposure was seen in DB versus NZW rabbits on Day 1 with minor accumulation of exposure with repeat dosing. Table 27 shows the Vitreous Humor pharmacokinetic parameters.

TABLE 27
Mean Vitreous Humor Pharmacokinetic Parameters of Compound
1 in New Zealand White and Dutch Belted Rabbits

| | | | Days of | T_{1/2} | Cₘₐₓ | Tₗₐₛₜ | AUCₗₐₛₜ | MRTₗₐₛₜ |
|---|---|---|---|---|---|---|---|---|
| Species | Dose (mg/kg) | | Dosing | (h) | (ng/mL) | (h) | (h*ng/mL) | (h) |
| New Zealand | 15 | single dose | 1 | NC^{a} | 7.61 | 6 | 30.3 | 2.24 |
| White | | | | | | | | |
| Dutch Belted | 15 | single dose | 1 | NC^{a} | 7.26 | 6 | 30.9 | 2.48 |
| Dutch Belted | 15 | BID | 15 | NC^{a} | 15.4 | 6 | 64.0 | 2.23 |
| Dutch Belted | 50 | BID | 15 | 5.44 | 49.2 | 24 | 419 | 7.27 |

^{a}NC: Not Calculated

As expected, the ratio of Choroid-RPE versus retinais ˜1 in NZW rabbits, which confirms that the lipophilic small molecule distributes to tissues readily. The ratio of Choroid-RPE versus retina is higher in DB rabbits, which is ˜20 fold at the trough and lower around Cₘₐₓ. The concentrations were between 15 and 50 mg BID groups in DB at the terminal phase (96-240 hr post last-dose). The concentrations are similar in both tissues (Choroid-RPE and retina). The concentration declines at a similar rate in Choroid-RPE, whereas it maintains at a similar level in the retina, which presumably comes from the depot. The mean Choroid-RPE and retina concentrations are shown in Table 28.

TABLE 28
Mean Choroid-RPE and Retina Concentrations of Compound 1 in New Zealand White and Dutch Belted Rabbits

| | Mean Choroid-RPE & Retina Concentration | | | | | | | | | | | |
|---|---|---|---|---|---|---|---|---|---|---|---|---|
| | | | | | | | DB Day 15 | | | | | |
| | NZW Day 1 | | | DB Day 1 | | | 15 mg/kg BID | | | DB Day 15 | | |
| | 15 mg/kg single dose | | | 15 mg/kg single dose | | | Choroid- | | | 50 mg/kg BID | | |
| Time | Choroid- | Retina | | Choroid- | Retina | | RPE | Retina | | Choroid- | Retina | |
| (hr) | RPE ng/g | ng/g | Ratio | RPE ng/g | ng/g | Ratio | ng/g | ng/g | Ratio | RPE ng/g | ng/g | Ratio |
| 0 | BLQ | BLQ | NC | BLQ | BLQ | NC | 2460 | 114 | 22 | 3918 | 159 | 25 |
| 1 | 172 | 200 | 1 | 917 | 145 | 6 | 4870 | 576 | 8 | 8025 | 2122 | 4 |
| 6 | 31.5 | 26 | 1 | 866 | 29.6 | 29 | 3200 | 237 | 14 | 4373 | 725 | 6 |
| 24 | 15.1 | BLQ | NC | 311 | 24.7 | 13 | 1960 | 74.3 | 26 | 2097 | 265 | 8 |
| 96 | BLQ | BLQ | NC | 77.5 | BLQ | NC | 866 | 28.1 | 31 | 873 | 32.3 | 27 |
| 168 | BLQ | BLQ | NC | 56.3 | BLQ | NC | 485 | 36.5 | 13 | 645 | 54.9 | 12 |
| 240 | BLQ | BLQ | NC | 28.1 | BLQ | NC | 340 | 25.3 | 13 | 195 | 39.4 | 5 |

BLQ: Below Limit of Quantitation;
NC, not calculated

In summary, plasma exposure profiles were similar on Day 1 in NZW and DB rabbits indicating that there is no exposure difference between the two strains. There was approximately 2× accumulation in plasma exposure upon repeat dosing. Generally, the exposure profiles of NZW and DB rabbit retina were similar to the exposure profile exhibited in plasma through 24 hours post-dose on Day 1 and Day 15, however DB retina shows prolonged exposure out to 240 hours post-dose on Day 15. Choroid-RPE and iris-ciliary body have significantly higher exposure profiles than plasma, and show significant concentrations out to 240 hours post-dose on Day 1 and Day 15. DB rabbits on Day 1 show significantly greater exposure than NZW rabbits on Day 1. The apparent accumulation of Compound 1 in Choroid-RPE and in iris-ciliary body demonstrates dose accumulation in tissues, even while plasma shows little to no dose accumulation. This shows that Compound 1 is able to bind to melanin in eye tissues, leading to higher concentrations in these tissues. The bound Compound 1 is able to be released from the melanin-containing cells, replenishing the concentration in the retina. The capacity of the drug depot is saturable. Vitreous humor exposures were similar on Day 1 in NZW and DB rabbits. After repeat dosing, minor accumulation was observed.

### Example 6. A Phase 1, Single Ascending Dose Study to Assess the Safety, Tolerability, Pharmacokinetics, and Pharmacodynamics of Compound 1 in Healthy Volunteers

This study was conducted in healthy human volunteers in a randomized, double-blind design. Compound 1 or placebo was administered as a single oral dose to twenty-eight healthy volunteers in three separate dose groups. Doses administered in the first three groups were 200 mg Compound 1 or placebo once daily (QD) (Group 1), 600 mg Compound 1 or placebo once daily (QD) (Group 2), and 1200 mg Compound 1 or placebo once daily (QD) (Group 3).

For all subjects, blood samples were collected at specific, protocol-defined timepoints from Day 1 to Day 4 to determine plasma concentrations of Compound 1 and any potential metabolites. Plasma pharmacokinetic parameters of Compound 1, including tₘₐₓ, Cₘₐₓ, and AUC were determined at each dose level using validated bioanalytical methods. Quantifiable concentrations of Compound 1 were observed for most of the post-treatment samples collected. Pharmacokinetic data from each volunteer is summarized in Table 29, and plasma concentration data is shown graphically in FIG. 19.

The mean Cₘₐₓ, mean AUC₍₀₋₂₄₎, and mean AUC_{(0-∞)} for Group 1 were 1059 ng/mL, 2391 ng*hr/mL, and 3015 ng*hr/mL, respectively. The mean Cₘₐₓ, mean AUC₍₀₋₂₄₎, and mean AUC_{(0-∞)} for Group 2 were 2022 ng/mL, 6189 ng*hr/mL, and 7093 ng*hr/mL, respectively. The mean Cₘₐₓ, mean AUC₍₀₋₂₄₎, and mean AUC_{(0-∞)} for Group 3 were 3068 ng/mL, 11427 ng*hr/mL, and 13163 ng*hr/mL, respectively.

TABLE 29
Pharmacokinetic Data of Single Ascending
Doses of Compound 1 in Healthy Volunteers

| | | Cₘₐₓ | Tₘₐₓ | AUC₍₀₋₂₄₎ | AUC_{(0-∞)} |
|---|---|---|---|---|---|
| | Subject | ng/mL | hr | ng · hr/mL | ng · hr/mL |
| Group 1 | 001-001 | 1380 | 1.50 | 3615.79 | 3822.48 |
| (200 mg | 001-006 | 635 | 2.50 | 1888.95 | 1993.07 |
| Single | 001-010 | 562 | 1.50 | 2229.07 | 2385.64 |
| Dose) | 001-011 | 950 | 1.50 | 3387.20 | 3616.53 |
| | 001-012 | 2060 | 1.25 | 4128.01 | 4241.37 |
| | 001-017 | 764 | 2.50 | 2339.30 | 2569.69 |
| | Mean | 1058.50 | 1.79 | 2931.39 | 3104.80 |
| | SD | 571.09 | 0.56 | 898.73 | 906.44 |
| | % CV | 54 | 31 | 31 | 29 |
| | N | 6 | 6 | 6 | 6 |
| | Min | 562.00 | 1.25 | 1888.95 | 1993.07 |
| | Max | 2060.00 | 2.50 | 4128.01 | 4241.37 |
| Group 2 | 001-021 | 2180 | 1.50 | 7017.08 | 8156.63 |
| (600 mg | 001-022 | 2480 | 2.00 | 6591.55 | 6805.91 |
| Single | 001-026 | 2180 | 1.00 | 6135.83 | 7335.98 |
| Dose) | 001-028 | 2100 | 1.50 | 6020.33 | 6903.06 |
| | 001-030 | 1380 | 1.25 | 4079.30 | 4687.40 |
| | 001-033 | 1810 | 2.50 | 7288.50 | 8671.53 |
| | Mean | 2021.67 | 1.63 | 6188.76 | 7093.42 |
| | SD | 380.23 | 0.54 | 1143.57 | 1384.74 |
| | % CV | 19 | 33 | 18 | 20 |
| | N | 6 | 6 | 6 | 6 |
| | Min | 1380.00 | 1.00 | 4079.30 | 4687.40 |
| | Max | 2480.00 | 2.50 | 7288.50 | 8671.53 |
| Group 3 | 001-035 | 3080 | 2.5 | 12680.35 | 15841.50 |
| (1200 mg | 001-041 | 3230 | 2.5 | 11822.00 | 13297.29 |
| Single | 001-042 | 3530 | 2.5 | 10219.40 | 10982.71 |
| Dose) | 001-045 | 1940 | 2.5 | 8431.90 | 10243.93 |
| | 001-052 | 2530 | 2.5 | 11357.50 | 13190.18 |
| | 001-058 | 4100 | 2.5 | 14054.00 | 15424.93 |
| | Mean | 3068.33 | 2.50 | 11427.53 | 13163.42 |
| | SD | 757.19 | 0.00 | 1952.53 | 2262.04 |
| | % CV | 25 | 0 | 17 | 17 |
| | N | 6 | 6 | 6 | 6 |
| | Min | 1940.00 | 2.50 | 8431.90 | 10243.93 |
| | Max | 4100.00 | 2.50 | 14054.00 | 15841.50 |

Compound 1 was readily absorbed, with median time to maximum ding concentration (Tₘₐₓ) of 1.0 to 2.3 hours after dosing in all individuals across the studied dose range (200 to 800 mg BID and 75 mg TID). Steady state conditions of Compound 1 were reached on Day 2. This MAD study showed that the PK of Compound 1 are linear over the dose range of 200 to 800 mg BID. The PR of Compound 1 in humans behaved in a predictable manner with Cₘₐₓ and AUCₜₐᵤ increasing approximately dose proportionally (linearly) with increasing dose.

### Example 7. Ocular Tissue Distribution and Pharmacokinetics of Orally Administered Compound 1 in Pigmented (Dutch Belted) and Nonpigmented (New Zealand White) Rabbits

A study was conducted to assess the pharmacokinetics and ocular tissue distribution of Compound 1 follow oral administration to pigmented (Dutch Belted) and nonpigmented (New Zealand White Rabbits). A summary of the study design is provided in Table 30 below:

TABLE 30
Group Designations and Dose Information

| | | | | Target Dose | | |
|---|---|---|---|---|---|---|
| | Number | | Dose | Level | Target Dose | |
| | of Male | Dose | Concentration | (mg/kg/dose)/ | Volume | |
| Group | Animals | Route | (mg/mL) | (mg/kg/day) | (mL/kg/dose) | Sample Collections |
| 1 | 14 DB | Oral^{a} | 1.5 | 7.5/7.5 | 5 | Blood and Ocular tissues |
| 2 | 15 DB | Oral^{b} | 1.5 | 7.5/15 | 5 | Blood and Ocular tissues |
| 3 | 15 DB | Oral^{b} | 3 | 15/30 | 5 | Blood and Ocular tissues |
| 4 | 15 NZW | Oral^{b} | 3 | 15/30 | 5 | Blood and Ocular tissues |

DB Dutch Belted.
NZW New Zealand White.
Note:
One animal/group for Groups 2, 3, and 4 served as a dosed extra and was available as a replacement to meet study requirements as necessary.
^{a}Animals received a single dose on Day 1.
^{b}Animals received a twice daily (BID) dose (approximately 12 hours apart) for 14 days, with a single a.m. dose on Day 15 (for a total of 29 doses). Doses were administered at approximately the same times each day (±1 hour).

Following a single administration or repeat oral administrations of Compound 1, animals were sacrificed, and blood (centrifuged to prepare plasma) and ocular tissues (choroid-retinal pigment epithelium [choroid-RPE], iris-ciliary body [ICB], and retina) were collected as described below.

### Sample Collection

### Blood

For Group 1, two animals/time point were sacrificed via an overdose of sodium pentobarbital at approximately 1, 6, 12, 24, 96, 168, and 240 hours postdose. Blood (approximately 2 mL) was collected from all animals at sacrifice into tubes containing K₂EDTA via exsanguination (cardiac puncture).

For Groups 2 through 4, blood (approximately 2 mL) was collected via a jugular vein using a syringe and needle and was transferred into tubes containing K₂EDTA at approximately 12 hours after the a.m. dose on Day 15 from the two animals designated for sacrifice at approximately 240 hours after the a.m. dose on Day 15.

For Groups 2 through 4, two animals/group/time point were sacrificed via an overdose of sodium pentobarbital prior to dosing on Day 15 and at approximately 1, 6, 24, 96, 168, and 240 hours after the a.m. dose on Day 15. Blood (approximately 2 mL) was collected from all animals at sacrifice into tubes containing K₂EDTA via exsanguination (cardiac puncture).

Blood was maintained on wet ice, in chilled cryoracks, or at approximately 5° C. prior to centrifugation to obtain plasma. Centrifugation began within 1 hour of collection (Deviation). Plasma was placed into 96-well tubes with barcode labels. Plasma was maintained on dry ice prior to storage at approximately −70° C.

### Ocular Samples

At the time of sacrifice, both eyes were enucleated, and the following tissues were collected. Each eye was flash-frozen in liquid nitrogen for 15 to 20 seconds. The enucleated eye was placed on dry ice or stored at approximately −70° C. for at least 2 hours. Within approximately 3 days, the frozen matrices were collected as right and left eyes for each matrix into the specific tube type listed.

| Frozen Collection | Collection Tube Requirements |
|---|---|
| Choroid - RPE | 2 mL OMNI tubes with lysing matrix |
| Iris-ciliary body (ICB) | 7 mL OMNI tubes with lysing matrix |
| Retina^{a} | 2 mL OMNI tubes with lysing matrix |

RPE Retinal pigment epithelium.
^{a}Filter paper was used to collect the retina.

At the time of frozen collection, the ocular tissues were rinsed with saline and blotted dry (as appropriate), weighed, and placed on dry ice. All ocular tissues were collected as single samples.

### Results

The single oral administration (7.5 mg/kg/day) and the twice daily (BID) administrations (≥15 mg/kg/day) of Compound 1 were well tolerated, and no Compound 1-related clinical observations were noted.

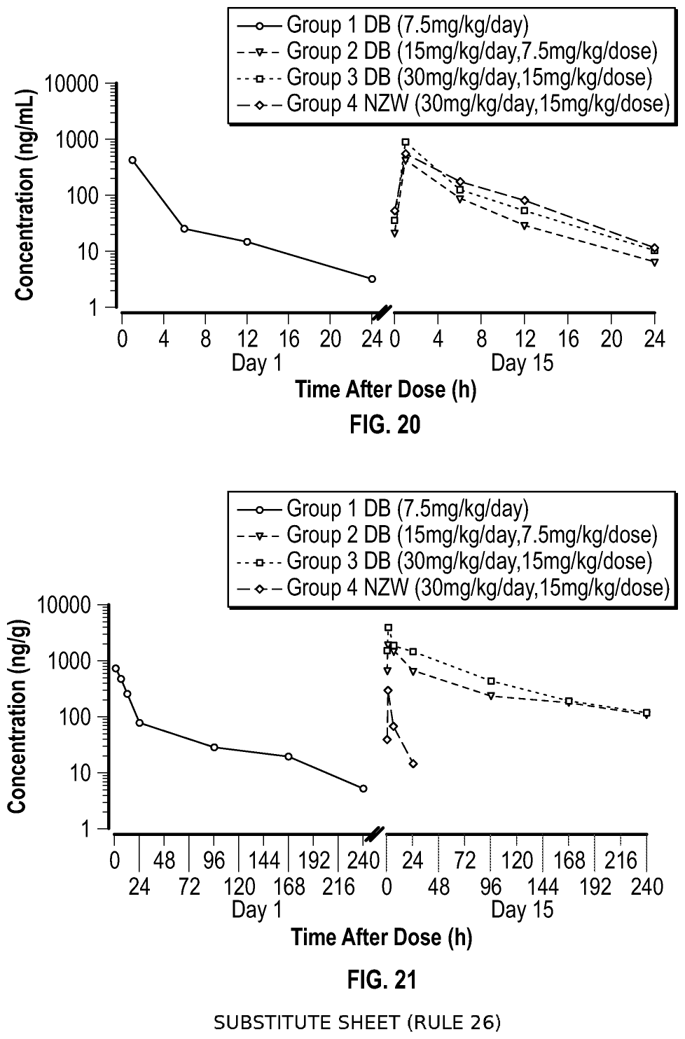

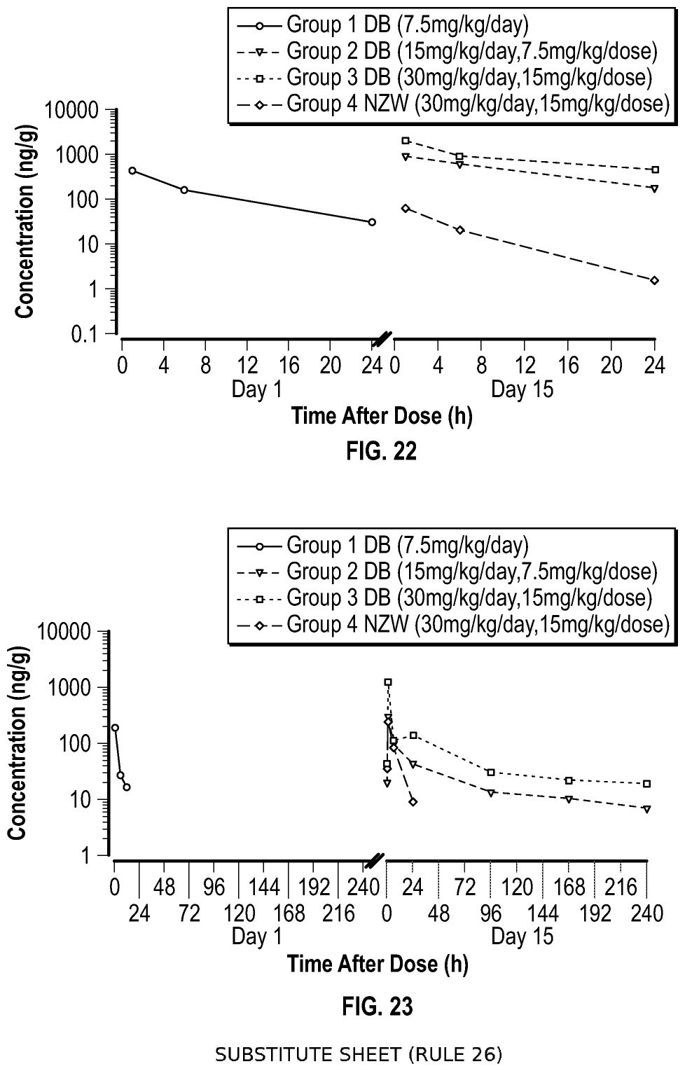

After a single oral dose to Dutch Belted (DB) rabbits or BID oral doses to DB and New Zealand White (NZW) rabbits, Compound 1 was absorbed into plasma and ocular tissues (choroid-RPE, ICB, and retina). In DB rabbits, the highest concentrations of Compound 1 in tissues was observed in the melanin containing choroid-RPE and ICB, followed by the non-melanin containing retina. Concentrations were measurable in the choroid-RPE and ICB through the last collection time point, 240 and 24 hours postdose, respectively, and through 240 hours postdose in the retina following a repeated 15 or 30 mg/kg/day dose and 24 hours postdose following a single 7.5 mg/kg dose. For DB rabbits that received a single administration of ALXN-2040 or a repeated administration at a dose level of 15 mg/kg/day, the concentrations in the choroid-RPE and ICB were higher than those observed in plasma and for the retina were generally comparable. Following a repeated administration of Compound 1 at a dose level of 30 mg/kg/day, the concentrations in ocular tissues were higher than those in plasma. In NZW rabbits, the concentrations of Compound 1 in tissues were generally comparable to those in plasma. Compound 1 concentrations in tissues were measurable through 24 hours postdose and highest in the retina. Following 15 days repeated administration to DB rabbits, Compound 1 exposure (as assessed by Cm, and AUC) in plasma and ocular tissues increased with the increase in dose level, from 15 to 30 mg/kg/day. The increases in Cₘₐₓ and AUC values were generally dose proportional in plasma, choroid-RPE, and ICB, but were greater than proportional in the retina. At 30 mg/kg/day (15 mg/kg/dose, BID), plasma exposure was comparable for DB and NZW rabbits. Ocular tissue exposure was higher in the melanin-containing tissues (ICB and choroid-RPE) from DB rabbits compared to NZW rabbits, with differences generally greater than 10-fold, indicating melanin-binding occurred. Mean plasma and tissue exposure is shown in FIGS. 20-23 and Table 31-34, and mean pharmacokinetic parameters of Compound 1 are summarized in Tables 35-38.

TABLE 31
Mean Plasma Concentrations from New Zealand
White and Dutch Belted Rabbits

| | Mean Plasma Concentration [ng/mL] | | | |
|---|---|---|---|---|
| | DB Day 1 | DB Day 15 | DB Day 15 | NZW Day 15 |
| | 7.5 mg/kg | 7.5 mg/kg | 15 mg/kg | 15 mg/kg |
| Time (hr) | single dose | BID | BID | BID |
| 0 | NR | 22.2 | 36.4 | 53.8 |
| 1 | 423 | 440 | 910 | 558 |
| 6 | 25.1 | 90.1 | 130 | 179 |
| 12 | 14.8 | 29.6 | 54.3 | 82.2 |
| 24 | 3.26 | 6.64 | 10.5 | 11.7 |
| 96 | BLQ | BLQ | BLQ | BLQ |
| 168 | BLQ | BLQ | BLQ | BLQ |
| 240 | BLQ | BLQ | BLQ | BLQ |

BLQ: Below Limit of Quantitation;
NR, not reported

TABLE 32
Mean Choroid-RPE Concentrations of Compound 1
in New Zealand White and Dutch Belted Rabbits

| | Mean Choroid-RPE Concentration | | | | | | | |
|---|---|---|---|---|---|---|---|---|
| | DB Day 1 | | DB Day 15 | | DB Day 15 | | NZW Day 15 | |
| | 7.5 mg/kg single dose | | 7.5 mg/kg BID | | 15 mg/kg BID | | 15 mg/kg BID | |
| | | vs. | | vs. | | vs. | | vs. |
| Time (hr) | ng/g | Plasma | ng/g | Plasma | ng/g | Plasma | ng/g | Plasma |
| 0 | NR | NC | 695 | 31.3 | 1540 | 42.3 | 40.3 | 0.75 |
| 1 | 726 | 1.72 | 2000 | 4.54 | 3970 | 4.36 | 296 | 0.53 |
| 6 | 472 | 18.8 | 1530 | 17.0 | 1880 | 14.5 | 68.6 | 0.83 |
| 12 | 260 | 17.6 | NR | NC | NR | NC | NR | NC |
| 24 | 78.0 | 23.9 | 685 | 103 | 1470 | 140 | 14.9 | 1.27 |
| 96 | 28.4 | NC | 238 | NC | 441 | NC | BLQ | NC |
| 168 | 19.4 | NC | 183 | NC | 195 | NC | BLQ | NC |
| 240 | 5.23 | NC | 111 | NC | 119 | NC | BLQ | NC |

BLQ: Below Limit of Quantitation;
NR, not reported;
NC, not calculated

TABLE 33
Mean Iris-Ciliary Body Concentrations of Compound
1 in New Zealand White and Dutch Belted Rabbits

| | Mean Iris-Ciliary Concentration | | | | | | | |
|---|---|---|---|---|---|---|---|---|
| | DB Day 1 | | DB Day 15 | | DB Day 15 | | NZW Day 15 | |
| | 7.5 mg/kg single dose | | 7.5 mg/kg BID | | 15 mg/kg BID | | 15 mg/kg BID | |
| | | vs. | | vs. | | vs. | | vs. |
| Time (hr) | ng/g | Plasma | ng/g | Plasma | ng/g | Plasma | ng/g | Plasma |
| 0 | NR | NC | NR | NC | NR | NC | NR | NC |
| 1 | 435 | 1.03 | 943 | 2.14 | 2050 | 2.25 | 64.9 | 0.12 |
| 6 | 163 | 6.49 | 634 | 7.04 | 939 | 7.22 | 21.2 | 0.12 |
| 12 | NR | NC | NR | NC | NR | NC | NR | NC |
| 24 | 31.4 | 9.63 | 184 | 27.7 | 460 | 43.8 | 1.60 | 0.14 |
| 96 | NR | NC | NR | NC | NR | NC | NR | NC |
| 168 | NR | NC | NR | NC | NR | NC | NR | NC |
| 240 | NR | NC | NR | NC | NR | NC | NR | NC |

BLQ: Below Limit of Quantitation;
NR, not reported;
NC, not calculated

TABLE 34
Mean Retina Concentrations of Compound 1 in
New Zealand White and Dutch Belted Rabbits

| | Mean Retina Concentration | | | | | | | |
|---|---|---|---|---|---|---|---|---|
| | DB Day 1 | | DB Day 15 | | DB Day 15 | | NZW Day 15 | |
| | 7.5 mg/kg single dose | | 7.5 mg/kg BID | | 15 mg/kg BID | | 15 mg/kg BID | |
| | | vs. | | vs. | | vs. | | vs. |
| Time (hr) | ng/g | Plasma | ng/g | Plasma | ng/g | Plasma | ng/g | Plasma |
| 0 | NR | NC | 20.7 | NC | 45.0 | 1.18 | 36.3 | 0.67 |
| 1 | 188 | 0.44 | 306 | 0.70 | 1123 | 1.23 | 248 | 0.44 |
| 6 | 27.5 | 1.10 | 97.9 | 1.09 | 115 | 0.88 | 86.1 | 0.48 |
| 12 | 16.5 | 1.11 | NR | NC | NR | NC | NR | NC |
| 24 | BLQ | NC | 44.3 | 6.67 | 139 | 13.2 | 9.18 | 0.62 |
| 96 | BLQ | NC | 13.9 | NC | 31.4 | NC | BLQ | NC |
| 168 | BLQ | NC | 10.7 | NC | 22.5 | NC | BLQ | NC |
| 240 | BLQ | NC | 7.18 | NC | 19.7 | NC | BLQ | NC |

BLQ: Below Limit of Quantitation;
NR, not reported;
NC, not calculated

TABLE 35
Summary of the Mean Pharmacokinetic Parameters for Compound 1 in Male
Rabbit Plasma Following Single or Repeated (BID) Oral Administration

| | | | | | | DN | | | | | DN | | DN |
|---|---|---|---|---|---|---|---|---|---|---|---|---|---|
| | | | Dose | Dose | | Cₘₐₓ | | | | | AUC₀₋₁₂ | | AUC₀₋₂₄ |
| | | | Level | Level | Cₘₐₓ | [(ng/mL)/ | C_{trough} | | AUC₀₋ₜ | AUC₀₋₁₂ | [(h*ng/mL)/ | AUC₀₋₂₄ | [(h*ng/mL)/ |
| Interval | | Dose | (mg/kg/ | (mg/kg/ | (ng/ | (mg/kg/ | (ng/ | Tₘₐₓ | (h*ng/ | (h*ng/ | (mg/kg/ | (h*ng/ | (mg/kg/ |
| (Day) | Strain | Group | day) | dose) | mL) | day) | mL) | (h) | mL) | mL) | dose)] | mL) | day) |
| 1 | DB | 1 | 7.5 | 7.5 | 423 | 56.4 | 14.8 | 1.00 | 1560 | 1450 | 194 | 1560 | 208 |
| 15 | DB | 2 | 15 | 7.5 | 440 | 58.6 | 29.6 | 1.00 | 2130 | 1910 | 255 | 2130 | 284 |
| | DB | 3 | 30 | 15 | 910 | 60.7 | 54.3 | 1.00 | 4010 | 3620 | 242 | 4010 | 267 |
| | NZW | 4 | 30 | 15 | 558 | 37.2 | 82.2 | 1.00 | 3490 | 2930 | 195 | 3490 | 233 |

| | | | | | | DN | | | |
|---|---|---|---|---|---|---|---|---|---|
| | | | Dose | Dose | | AUC₀₋₂₄₀ | | | |
| | | | Level | Level | AUC₀₋₂₄₀ | [(h*ng/mL)/ | AUC_{0-inf} | | |
| Interval | | Dose | (mg/kg/ | (mg/kg/ | (h*ng/ | (mg/kg/ | (h*ng/ | t_{1/2} | MRTₗₐₛₜ |
| (Day) | Strain | Group | day) | dose) | mL) | day) | mL) | (h) | (h) |
| 1 | DB | 1 | 7.5 | 7.5 | 1680 | 224 | 1590 | 6.01 | 2.67 |
| 15 | DB | 2 | 15 | 7.5 | 2370 | 316 | NR | 4.88 | NR |
| | DB | 3 | 30 | 15 | 4390 | 293 | NR | 4.98 | NR |
| | NZW | 4 | 30 | 15 | 3910 | 261 | NR | 4.53 | NR |

DB Dutch Belted.
NZW New Zealand White.
NR Not reported.
Note:
Group 1 received a single dose on Day 1. Groups 2 through 4 received twice daily doses, approximately 12 hours apart, on Days 1 through 14, followed by a single dose on Day 15.

TABLE 36
Summary of the Mean Pharmacokinetic Parameters for Compound 1 in Male Rabbit
Choroid-RPE Following Single or Repeated (Twice Daily) Oral Administration

| | | | | | | | DN | | | | | DN | | DN |
|---|---|---|---|---|---|---|---|---|---|---|---|---|---|---|
| | | | Dose | Dose | | | Cₘₐₓ | | | | | AUC₀₋₁₂ | | AUC₀₋₂₄ |
| | | | Level | Level | | Cₘₐₓ | [(ng/g)/ | C_{trough} | | AUC₀₋ₜ | AUC₀₋₁₂ | [(h*ng/g)/ | AUC₀₋₂₄ | [(h*ng/g)/ |
| Interval | | Dose | (mg/kg/ | (mg/kg/ | | (ng/ | (mg/kg/ | (ng/ | Tₘₐₓ | (h*ng/ | (h*ng/ | (mg/kg/ | (h*ng/ | (mg/kg/ |
| (Day) | Strain | Group | day) | dose) | Eye | g) | day | g) | (h) | g) | g) | dose)] | g) | day) |
| 1 | DB | 1 | 7.5 | 7.5 | OU | 726 | 96.8 | 260 | 1.00 | 14000 | 5550 | 740 | 7580 | 1010 |
| 15 | DB | 2 | 15 | 7.5 | OU | 2000 | 267 | 1250 | 1.00 | 89100 | 18500 | 2470 | 30100 | 4020 |
| | DB | 3 | 30 | 15 | OU | 3970 | 265 | 1740 | 1.00 | 151000 | 28200 | 1880 | 47500 | 3170 |
| | NZW | 4 | 30 | 15 | OU | 296 | 19.8 | 50.7 | 1.00 | 1830 | 1440 | 95.9 | 1830 | 122 |

| | | | | | | | DN | | | |
|---|---|---|---|---|---|---|---|---|---|---|
| | | | Dose | Dose | | | AUC₀₋₂₄₀ | | | |
| | | | Level | Level | | AUC₀₋₂₄₀ | [(h*ng/g)/ | AUC_{0-inf} | | |
| Interval | | Dose | (mg/kg/ | (mg/kg/ | | (h*ng/ | (mg/kg/ | (h*ng/ | t_{1/2} | MRTₗₐₛₜ |
| (Day) | Strain | Group | day) | dose) | Eye | g) | day) | g) | (h) | (h) |
| 1 | DB | 1 | 7.5 | 7.5 | OU | 14000 | 1870 | 14500 | 58.9 | 42.8 |
| 15 | DB | 2 | 15 | 7.5 | OU | 89100 | 11900 | NR | NR | NC |
| | DB | 3 | 30 | 15 | OU | 151000 | 10000 | NR | 76.2 | NC |
| | NZW | 4 | 30 | 15 | OU | 2370 | 158 | NR | NR | NC |

DB Dutch Belted.
NC Not calculated.
NR Not reported.
NZW New Zealand White.
Note:
Group 1 received a single dose on Day 1. Groups 2 through 4 received twice daily doses, approximately 12 hours apart, on Days 1 through 14, followed by a single dose on Day 15.

TABLE 37
Summary of the Mean Pharmacokinetic Parameters for Compound 1 in Male Rabbit
ICB Following Single or Repeated (Twice Daily) Oral Administration

| | | | | | | | DN | | | | | DN | | DN |
|---|---|---|---|---|---|---|---|---|---|---|---|---|---|---|
| | | | Dose | Dose | | | Cₘₐₓ | | | | | AUC₀₋₁₂ | | AUC₀₋₂₄ |
| | | | Level | Level | | Cₘₐₓ | [(ng/g)/ | C_{trough} | | AUC₀₋ₜ | AUC₀₋₁₂ | [(h*ng/g)/ | AUC₀₋₂₄ | [(h*ng/g)/ |
| Interval | | Dose | (mg/kg/ | (mg/kg/ | | (ng/ | (mg/kg/ | (ng/ | Tₘₐₓ | (h*ng/ | (h*ng/ | (mg/kg/ | (h*ng/ | (mg/kg/ |
| (Day) | Strain | Group | day) | dose) | Eye | g) | day) | g) | (h) | g) | g) | dose)] | g) | day) |
| 1 | DB | 1 | 7.5 | 7.5 | OU | 435 | 57.9 | 119 | 1.00 | 3460 | 2560 | 341 | 3460 | 462 |
| 15 | DB | 2 | 15 | 7.5 | OU | 943 | 126 | 484 | 1.00 | 11900 | 7860 | 1050 | 11900 | 1580 |
| | DB | 3 | 30 | 15 | OU | 2050 | 136 | 780 | 1.00 | 21300 | 13900 | 925 | 21300 | 1420 |
| | NZW | 4 | 30 | 15 | OU | 64.9 | 4.33 | 14.6 | 1.00 | 453 | 356 | 23.7 | 453 | 30.2 |

| | | | | | | | DN | | | |
|---|---|---|---|---|---|---|---|---|---|---|
| | | | Dose | Dose | | | AUC₀₋₂₄₀ | | | |
| | | | Level | Level | | AUC₀₋₂₄₀ | [(h*ng/g)/ | AUC_{0-inf} | | |
| Interval | | Dose | (mg/kg/ | (mg/kg/ | | (h*ng/ | (mg/kg/ | (h*ng/ | t_{1/2} | MRTₗₐₛₜ |
| (Day) | Strain | Group | day) | dose) | Eye | g) | day) | g) | (h) | (h) |
| 1 | DB | 1 | 7.5 | 7.5 | OU | NC | NC | NR | NR | 5.59 |
| 15 | DB | 2 | 15 | 7.5 | OU | NC | NC | NR | NR | NR |
| | DB | 3 | 30 | 15 | OU | NC | NC | NR | NR | NR |
| | NZW | 4 | 30 | 15 | OU | NC | NC | NR | NR | NR |

DB Dutch Belted.
NC Not calculated.
NR Not reported.
NZW New Zealand White.
Note:
Group 1 received a single dose on Day 1. Groups 2 through 4 received twice daily doses, approximately 12 hours apart, on Days 1 through 14, followed by a single dose on Day 15.

TABLE 38
Summary of the Mean Pharmacokinetic Parameters for Compound 1 in Male Rabbit
Retina Following Single or Repeated (Twice Daily) Oral Administration

| | | | | | | | DN | | | | | DN | | DN |
|---|---|---|---|---|---|---|---|---|---|---|---|---|---|---|
| | | | Dose | Dose | | | Cₘₐₓ | | | | | AUC₀₋₁₂ | | AUC₀₋₂₄ |
| | | | Level | Level | | | [(ng/g)/ | C_{trough} | | AUC₀₋ₜ | AUC₀₋₁₂ | [(h*ng/g)/ | AUC₀₋₂₄ | [(h*ng/g)/ |
| Interval | | Dose | (mg/kg/ | (mg/kg/ | | Cₘₐₓ | (mg/kg/ | (ng/ | Tₘₐₓ | (h*ng/ | (h*ng/ | (mg/kg/ | (h*ng/ | (mg/kg/ |
| (Day) | Strain | Group | day) | dose) | Eye | (ng/g) | day) | g) | (h) | g) | g) | dose)] | g) | day) |
| 1 | DB | 1 | 7.5 | 7.5 | OU | 188 | 25.1 | 16.5 | 1.00 | 765 | 765 | 102 | 864 | 115 |
| 15 | DB | 2 | 15 | 7.5 | OU | 306 | 40.8 | 80.0 | 1.00 | 6070 | 1710 | 227 | 2450 | 327 |
| | DB | 3 | 30 | 15 | OU | 1290 | 85.7 | 123 | 1.00 | 16100 | 11500 | 325 | 6460 | 430 |
| | NZW | 4 | 30 | 15 | OU | 248 | 16.5 | 60.4 | 1.00 | 1830 | 1420 | 94.4 | 1830 | 122 |

| | | | | | | | DN | | | |
|---|---|---|---|---|---|---|---|---|---|---|
| | | | Dose | Dose | | | AUC₀₋₂₄₀ | | | |
| | | | Level | Level | | AUC₀₋₂₄₀ | [(h*ng/g)/ | AUC_{0-inf} | | |
| Interval | | Dose | (mg/kg/ | (mg/kg/ | | (h*ng/ | (mg/kg/ | (h*ng/ | t_{1/2} | MRTₗₐₛₜ |
| (Day) | Strain | Group | day) | dose) | Eve | g) | day) | g) | (h) | (h) |
| 1 | DB | 1 | 7.5 | 7.5 | OU | 864 | 115 | NR | NR | 2.70 |
| 15 | DB | 2 | 15 | 7.5 | OU | 6070 | 810 | NR | NR | NR |
| | DB | 3 | 30 | 15 | OU | 16100 | 1070 | NR | NR | NR |
| | NZW | 4 | 30 | 15 | OU | 2160 | 144 | NR | NR | NR |

DB Dutch Belted.
NZW New Zealand White.
Note:
Group 1 received a single dose on Day 1. Groups 2 through 4 received twice daily doses, approximately 12 hours apart, on Days 1 through 14, followed by a single dose on Day 15.

This data suggests that the melanin containing tissues may act as a reservoir for Compound 1 and contribute to the prolonged exposure in retina of DB rabbits.

### Example 8. In Vitro Binding of Compound 1 to Melanin

Compound 1 (MW 580.4, 99.2% purity) and chloroquine (positive control, Sigma-Aldrich) were tested for binding to two sources of melanin (both from Sigma-Aldrich): synthetic melanin and melanin from Sepia officinalis, at concentrations from 0.123 to 50.0 μM for Compound 1 and from 0.062 to 25.0 μM for the positive control.

Compound stock solutions were prepared in dimethyl sulfoxide and then further diluted in Phosphate Buffered Saline (PBS) with and without melanin to the specified concentrations and incubated at 37° C. for 1 hour. A stock solution of Compound 1 was prepared at 50 mM in DMSO and was diluted to 5 mM with DMSO. A stock solution of chloroquine was prepared at 5 mM in DMSO. Compound 1 and the chloroquine stock solutions were serially diluted with DMSO and PBS respectively, followed by a further 1:100 dilution into warm (37° C.) PBS to obtain 2× the assay concentrations: 0.0274, 0.082, 0.247, 0.74, 2.22, 4.44, 8.89, 13.3, 20.0, 30.0, 40.0, and 50.0 μM for the test article and chloroquine. Melanin was added to warm (37° C.) PBS at a concentration of 0.1 mg/mL (2× assay concentration), mixed, and sonicated in the dark until no suspended aggregates were visible. The melanin solution was stored in the dark at 37° C. until use.

Aliquots (200 μL, each) of 2× assay samples were then added to an equal volume of warm (37° C.) PBS with or without 0.1 mg/mL melanin (50 μg/mL final concentration) in a polypropylene 96-well plate. Immediately after the assay, samples were added to an equal volume of warm PBS, aliquots (3 μL each) were removed, added to 100 μL of acetonitrile containing carbutamide as internal standard, and then diluted with water (197 μL). These time-zero samples, prepared in PBS only, were also used as calibration standards.

The assay plates containing samples with and without melanin were incubated in the dark with shaking at 37° C. for 1 hour. Following incubation, the samples were centrifuged for 15 minutes at approximately 4000 rpm at 37° C. to sediment the melanin. Sample supernatant (3 μL) was added to 100 μL of acetonitrile containing the internal standard and then diluted with water (197 μL).

Following centrifugation, samples were analyzed by LC-MS/MS bioanalysis as follows: For Compound 1, a C18 reverse-phase column (Waters XSELECT HSS T3 2.5 μm, 50×2.1 mm) was used with a gradient elution (800 μL/min flow rate) starting at 99% mobile phase A (0.1% formic acid in water) to 95% mobile phase B (0.1% formic acid in acetonitrile). The column was set to a temperature of 55° C. For Chloroquine, a C18 reverse-phase column (Waters XSELECT HSS T3 2.5 μm, 30×2.1 mm) was used with a gradient elution (900 μL/min flow rate) starting at 99% mobile phase A (0.1% formic acid in water) to 95% mobile phase B (0.1% formic acid in acetonitrile). The column was set to a temperature of 55° C.

Analytes and internal standards were detected using an Applied Biosystems Sciex API-5500 triple quadrupole mass spectrometer with Agilent 1260 Infinity Binary Pump and Apricot Designs ADDA High-Speed Dual Arm Autosampling System. The instrument was equipped with an electrospray ionization source (600° C.) operated in the positive ion mode. Analytes and internal standards for all samples were monitored in the multiple-reaction-monitoring scan mode. The MRMs used were as follows:
Compound 1: 580.2/360.2
Chrysin (IS): 255.2/153.1
Chloroquine: 320.1/247.3
Carbutamide (IS): 272.1/156.0

Concentrations were back calculated using the assay PBS curve and used for binding and stability calculations.

### Data Collection and Analysis

Data was captured and processed using Analyst® Version 1.6.2. Data was analyzed, and results were calculated using Microsoft Excel using the following formulas:

Bound Conc. (μM)=Mean Total Conc. (μM)−Mean Free Conc. (μM)

Bound (nmol/mg melanin)=Bound Conc. (μM)/(Melanin Conc. (μg/mL)/1000)

% Bound=Bound Conc. (μM)/Total Conc. (μM)×100%

% Recovery=Mean Total Conc. (μM)/Nominal Conc. (μM)×100%

% Stability=Mean Total Conc. (μM) after 1 Hour/Mean Total Conc. (μM) at Time Zero×100%

Data sets were curve fit by plotting the bound concentration versus the free concentration 20 using GraphPad Prism v.5.0 (one-site hyperbolic model).

### Results and Discussion

LC-MS/MS ionization signal response correlated linearly for Compound 1 over a concentration range of 0.123 to 50.0 μM, and for chloroquine over a concentration range of 0.062 to 25.0 μM, in PBS matrix. Melanin binding results are shown in Table 39.

TABLE 39
Summary of Compound 1 Melanin Binding Results

| | | | Bₘₐₓ | Mean |
|---|---|---|---|---|
| | Melanin | K_{d} | (nmol/mg | Stability |
| Compound | Source | (μM) | melanin) | (%) |
| Compound 1 | Synthetic | 5.15 | 127 | 94.3 |
| | S. officinalis | 12.0 | 137 | |
| Chloroquine | Synthetic | 0.842 | 308 | 87.3 |
| | S. officinalis | 0.967 | 280 | |

Kd = Binding affinity;
Bmax = Binding capacity

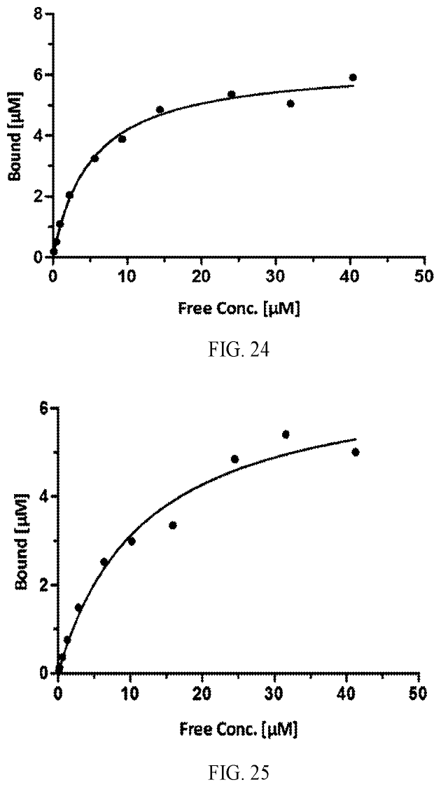

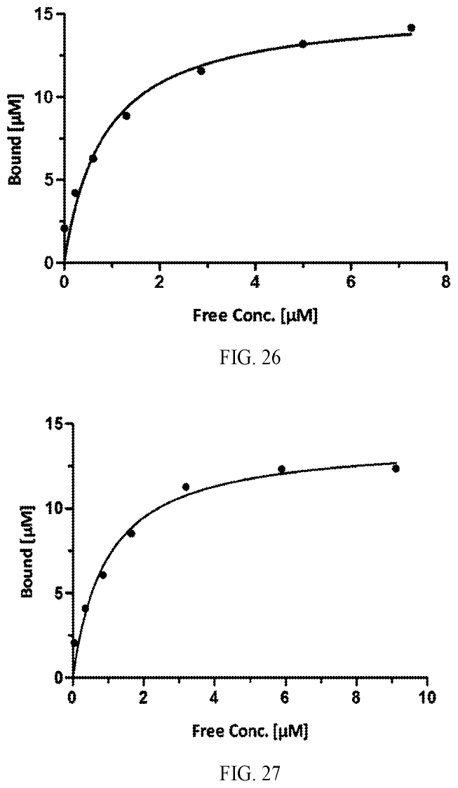

Compound 1 showed binding to melanin, the calculated affinity (K_{d}) was 5.15 μM and the binding capacity (Bₘₐₓ) was 127 nmol/mg melanin for synthetic melanin, and the calculated affinity (K_{d}) was 12.0 μM and the binding capacity (Bₘₐₓ) was 137 nmol/mg melanin for melanin from Sepia officinalis. Curves showing free versus bound Compound 1 to synthetic and Sepia officinalis melanin are shown in FIGS. 24 and 26 (synthetic) and FIGS. 25 and 27 (Sepia officinalis). Chloroquine, as the positive control, showed the expected binding to melanin. The chloroquine calculated affinity (K_{d}) was 0.842 μM and the binding capacity (Bₘₐₓ) was 308 nmol/mg melanin for synthetic melanin, and the calculated affinity (K_{d}) was 0.967 μM and the binding capacity (Bₘₐₓ) was 280 nmol/mg melanin for melanin from Sepia officinalis. The mean stability under the assay conditions were 94.3%. The results for the control, chloroquine, were within the expected range for the assay, confirming the validity of Compound 1 binding.

### Other Embodiments

This specification has been described with reference to certain embodiments and Examples. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of the claimed invention.

We claim:

1. A method for treating an alternative pathway complement D-mediated ocular disorder in a subject in need thereof, said method comprising systemically administering to the subject a compound of Formula I, or a pharmaceutically acceptable salt thereof, wherein:
(i) the compound of Formula I has the structure: 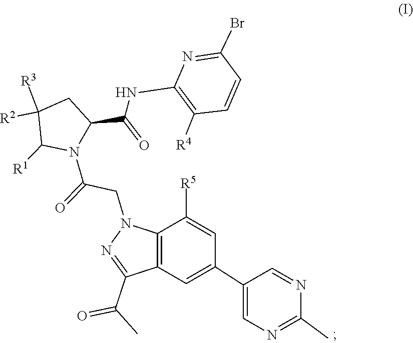
or an N-oxide, isotopic derivative, or prodrug thereof, and optionally in a pharmaceutically acceptable carrier to form a composition
wherein:
R¹ and R² are:
i. both hydrogen; or
ii. combined together to form a cyclopropyl ring;
R³ is hydrogen, methyl, or fluoro;
R⁴ is hydrogen or C₁-C₄ alkyl; and
R⁵ is hydrogen or methyl; and
(ii) a therapeutically effective amount of the compound of Formula I, or a pharmaceutically acceptable salt thereof, is systemically administered via oral or parenteral delivery to the subject.

2. The method of claim 1, wherein the ocular disorder is a posterior ocular disorder.

3. The method of claim 2, wherein the posterior ocular disorder is selected from the group consisting of dry and wet age-related macular degeneration (AMD), geographic atrophy, cytomegalovirus (CMV) infection, diabetic retinopathy, diabetic macular edema, choroidal neovascularization, acute macular neuroretinopathy, macular edema, Behcet's disease, retinal disorders, diabetic retinopathy, retinal arterial occlusive disease, central retinal vein occlusion, uveitic retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction, and retinitis pigmentosa.

4. The method of claim 3, wherein the posterior ocular disorder is geographic atrophy.

5. The method of claim 3, wherein the posterior ocular disorder is age-related macular degeneration.

6. The method of claim 5, wherein the posterior ocular disorder is dry age-related macular degeneration.

7. The method of claim 5, wherein the posterior ocular disorder is wet age-related macular degeneration.

8. The method of claim 3, wherein the posterior ocular disorder is macular edema selected from cystoid macular edema and diabetic macular edema.

9. The method of claim 3, wherein the posterior ocular disorder is diabetic retinopathy or proliferative diabetic retinopathy.

10. The method of claim 1, wherein the ocular disorder is an anterior ocular disorder.

11. The method of claim 10, wherein the anterior ocular disorder is selected from glaucoma, allergic conjunctivitis, anterior uveitis, and cataracts.

12. The method of claim 10, wherein the anterior ocular disorder is glaucoma.

13. The method of claim 10, wherein the anterior ocular disorder is allergic conjunctivitis.

14. The method of claim 10, wherein the anterior ocular disorder is anterior uveitis.

15. The method of claim 10, wherein the anterior ocular disorder is cataracts.

16. The method of any one of claims 1-15, wherein the systemic administration of the compound of Formula I, or a pharmaceutically acceptable salt thereof, results in an accumulation of the compound of Formula I, or a pharmaceutically acceptable salt thereof, in a melanin-containing ocular tissue of the subject, forming a depot that provides extended delivery of the compound of Formula I to an ocular tissue of the subject for at least one week, two weeks, or three weeks after the administration.

17. The method of claim 16, wherein the melanin-containing ocular tissue is choroid-retina pigmented epithelium (C-RPE) and/or iris ciliary body (I-CB).

18. The method of any one of claims 1-17, wherein the compound of Formula I, or a pharmaceutically acceptable salt thereof, accumulates at a ratio of at least 2× in the choroidal tissue of the eye versus in the plasma such that a depot is formed in the choroidal tissue of the subject which provides extended delivery of the compound of Formula I, or a pharmaceutically acceptable salt thereof, for at least 7 days, two weeks, three weeks or one month, two months, three months, four months, five months, or six months after cessation of administration of the compound of Formula I, or a pharmaceutically acceptable salt thereof.

19. The method of any one of claims 1-17, wherein the compound of Formula I, or a pharmaceutically acceptable salt thereof, accumulates at a ratio of at least 2× in the iris-ciliary body of the eye versus in the plasma such that a depot is formed in the iris-ciliary body of the subject which provides extended delivery of the compound of Formula I, or a pharmaceutically acceptable salt thereof, for at least 7 days, two weeks, three weeks or one month, two months, three months, four months, five months, or six months after cessation of administration of the compound of Formula I, or a pharmaceutically acceptable salt thereof.

20. The method of any one of claims 1-19, wherein the compound of Formula I, or a pharmaceutically acceptable salt thereof, is administered orally or parenterally once or twice a day.

21. The method of claim 20, wherein the compound of Formula I, or a pharmaceutically acceptable salt thereof, is administered orally once a day in a tablet, capsule, gel cap, or other solid dosage form.

22. The method of any one of claims 1-21, wherein the compound of Formula I, or a pharmaceutically acceptable salt thereof, is provided in a ramp-up dosage for a first period of time and then a lower maintenance dosage for a second period of time.

23. The method of claim 22, wherein the first period of time is at least, or no greater than, 5, 6, 7, 8, 9, 10, 11, 12, 13, 14 or 15 days.

24. The method of claim 22, wherein the second period of time is at least 5, 10, 15, 20, or 25 days or 1, 2, 3, 4, 5, or 6 months.

25. The method of any one of claims 1-24, wherein the compound of Formula I is Compound 1: 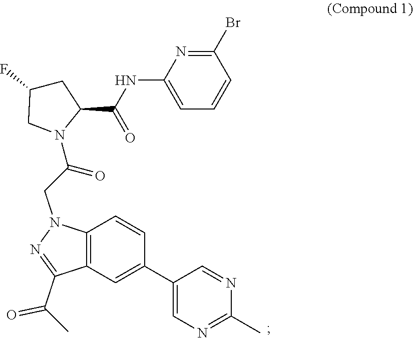 [SMILES: CC(=O)c1nn(CC(=O)N2C[C@H](F)C[C@H]2C(=O)Nc2cccc(Br)n2)c2ccc(-c3cnc(C)nc3)cc12]
or a pharmaceutically acceptable salt thereof.

26. The method of claim 25, wherein (a) the dosing regimen provides a plasma AUC₀₋₂₄ of between about 2500 ng*hr/mL and about 12000 ng*hr/mL; (b) the dosing regimen results in a depot of Compound 1 within an eye of the subject by day 15 of administration, and (c) by day 15, the ratio of Compound 1 in the retina of the eye compared to the plasma of the subject is maintained at no less than 0.60 throughout the dosing period.

27. The method of claim 26, wherein the dosing regimen results in a depot of Compound 1 in an eye of the subject by day 8, day 5, or day 3 of administration.

28. The method of any one of claims 25-27, wherein the ratio of Compound 1 in the retina of the eye compared to the plasma of the subject is maintained at no less than 0.65, no less than 0.70, or no less than 0.75 throughout the dosing period.

29. The method of claim 25, wherein (a) the dosing regimen provides a plasma AUC₀₋₂₄ of between about 2500 ng*hr/mL and about 12000 ng*hr/mL; (b) the dosing regimen results in a depot of Compound 1 within an eye of the subject by day 15 of administration, and (c) by day 15, the ratio of Compound 1 in the choroid-retina pigmented epithelium of the eye compared to the plasma is maintained at no less than 2.5× throughout the dosing period.

30. The method of claim 29, wherein the dosing regimen results in a depot of Compound 1 within an eye by day 8, by day 5, or by day 3 of administration.

31. The method of any one of claims 29-30, wherein the ratio of Compound 1 in the choroid-retina pigmented epithelium of the eye compared to the plasma is maintained between a range of 2.75 to 5.75 or greater throughout the dosing period.

32. The method of claim 25, wherein (a) the dosing regimen provides a plasma AUC₀₋₂₄ of between about 2500 ng*hr/mL and about 12000 ng*hr/mL; (b) the dosing regimen results in a depot of Compound 1 within an eye of the subject by day 15 of administration, and (c) by day 15, the ratio of Compound 1 in the iris-ciliary body of the eye compared to the plasma of the subject is maintained at no less than 3 throughout the dosing period.

33. The method of claim 32, wherein by day 8, the ratio of Compound 1 in the iris-ciliary body compared to the plasma is maintained between a range of 3 to 5.5 or greater throughout the dosing period.

34. The method of any one of claims 25-33, the method comprising orally administering Compound 1, or a pharmaceutically acceptable salt thereof, at a dosing regimen that provides a plasma AUC₀₋₂₄ of between about 2500 ng*hr/mL and about 12000 ng*hr/mL, wherein the dosing regimen results in a depot of Compound 1 within an eye of the subject by day 15 of administration, and wherein the depot contributes to the effective amount of Compound 1 contained in the eye throughout the dosing period.

35. The method of claim 34, wherein the dosing regimen results in a depot of Compound 1 within an eye by day 8 or by day 5 of administration.

36. The method of any one of claims 25-33, the method including orally administering Compound 1, or a pharmaceutically acceptable salt thereof, at a dosing regimen of from 200 mg to 1,000 mg, or from 200 mg to 800 mg, or 400 mg once a day.

37. The method of any one of claims 25-33, the method including orally administering Compound 1, or a pharmaceutically acceptable salt thereof, at a dosing regimen of from 50 mg to 250 mg twice a day, 100 mg twice a day, 200 mg twice a day, or 250 mg twice a day.

38. The method of any one of claims 1-24, wherein the compound of Formula I is Compound 2: 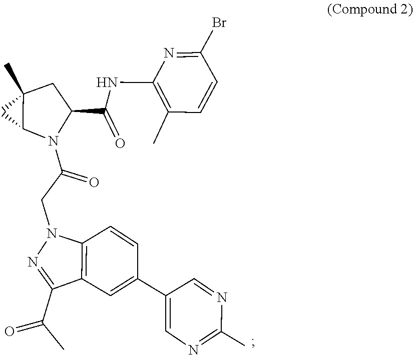 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof.

39. The method of claim 38, wherein (a) the dosing regimen provides a plasma AUC₀₋₂₄ of between about 500 ng*hr/ml and 4,500 ng*hr/ml; (b) the dosing regimen results in a depot of Compound 2 within an eye of the subject by day 15 of administration, and (c) by day 15, the ratio of a compound of Compound 2 in the retina of the eye compared to the plasma of the subject is maintained at no less than 0.60 throughout the dosing period.

40. The method of claim 39, wherein the dosing regimen results in a depot of Compound 2 in an eye of the subject by day 8, day 5, or day 3 of administration.

41. The method of any one of claims 38-40, wherein the ratio of Compound 2 in the retina of the eye compared to the plasma of the subject is maintained at no less than 0.65, no less than 0.70, or no less than 0.75 throughout the dosing period.

42. The method of claim 38, wherein (a) the dosing regimen provides a plasma AUC₀₋₂₄ of between about 500 ng*hr/ml and 4,500 ng*hr/ml; (b) the dosing regimen results in a depot of Compound 2 within an eye of the subject by day 15 of administration, and (c) by day 15, the ratio of Compound 2 in the choroid-retina pigmented epithelium of the eye compared to the plasma is maintained at no less than 2.5× throughout the dosing period.

43. The method of claim 42, wherein the dosing regimen results in a depot of Compound 2 within an eye by day 8, by day 5, or by day 3 of administration.

44. The method of any one of claims 38-43, wherein the ratio of Compound 2 in the choroid-retina pigmented epithelium of the eye compared to the plasma is maintained between a range of 2.75 to 5.75 or greater throughout the dosing period.

45. The method of claim 38, wherein (a) the dosing regimen provides a plasma AUC₀₋₂₄ of between about 500 ng*hr/ml and 4,500 ng*hr/ml; (b) wherein the dosing regimen results in a depot of Compound 2 within an eye of the subject by day 15 of administration; and (c) by day 15, the ratio of Compound 2 in the iris-ciliary body of the eye compared to the plasma of the subject is maintained at no less than 3 throughout the dosing period.

46. The method of claim 45, wherein by day 8, the ratio of Compound 2 in the iris-ciliary body compared to the plasma is maintained between a range of 3 to 5.5 or greater throughout the dosing period.

47. The method of any one of claims 38-46, the method comprising orally administering Compound 2, or a pharmaceutically acceptable salt thereof, at a dosing regimen that provides a plasma AUC₀₋₂₄ of between about 500 ng*hr/ml and 4,500 ng*hr/ml; wherein the dosing regimen results in a depot of Compound 2 within an eye of the subject by day 15 of administration, and wherein the depot contributes to the effective amount of Compound 2 contained in the eye throughout the dosing period.

48. The method of claim 47, wherein the dosing regimen results in a depot of Compound 2 within an eye by day 8 or by day 5 of administration.

49. The method of any one of claims 38-48, the method including orally administering Compound 2, or a pharmaceutically acceptable salt thereof, at a dosing regimen of from 40 mg to 300 mg, 80 mg to 250 mg, or 100 mg to 200 mg once a day.

50. The method of any one of claims 38-48, the method including orally administering Compound 2, or a pharmaceutically acceptable salt thereof, at a dosing regimen of less than 200 mg, less than 100 mg once a day, or 150 mg twice a day.

51. A method for treating an alternative pathway complement D-mediated ocular disorder in a subject in need thereof, said method comprising systemically administering to the subject a compound of Formula II, or a pharmaceutically acceptable salt thereof, wherein:
(i) the compound of Formula II has the structure: 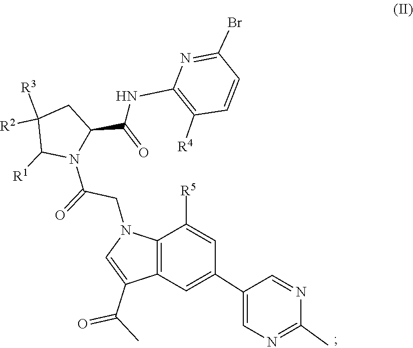
or an N-oxide, isotopic derivative, or prodrug thereof, and optionally in a pharmaceutically acceptable carrier to form a composition;
wherein:
R¹ and R² are:
i. both hydrogen; or
ii. combined together to form a cyclopropyl ring;
R³ is hydrogen, methyl, or fluoro;
R⁴ is hydrogen or C₁-C₄ alkyl; and
R⁵ is hydrogen or methyl; and
(ii) a therapeutically effective amount of the compound of Formula II, or a pharmaceutically acceptable salt thereof, is systemically administered via oral or parenteral delivery to the subject.

52. The method of claim 51, wherein the ocular disorder is a posterior ocular disorder.

53. The method of claim 52, wherein the posterior ocular disorder is selected from the group consisting of dry and wet age-related macular degeneration (AMD), geographic atrophy, cytomegalovirus (CMV) infection, diabetic retinopathy, diabetic macular edema, choroidal neovascularization, acute macular neuroretinopathy, macular edema, Behcet's disease, retinal disorders, diabetic retinopathy, retinal arterial occlusive disease, central retinal vein occlusion, uveitic retinal disease, retinal detachment, ocular trauma, damage caused by ocular laser treatment or photodynamic therapy, photocoagulation, radiation retinopathy, epiretinal membrane disorders, branch retinal vein occlusion, anterior ischemic optic neuropathy, non-retinopathy diabetic retinal dysfunction, and retinitis pigmentosa.

54. The method of claim 53, wherein the posterior ocular disorder is geographic atrophy.

55. The method of claim 53, wherein the posterior ocular disorder is age-related macular degeneration.

56. The method of claim 55, wherein the posterior ocular disorder is dry age-related macular degeneration.

57. The method of claim 55, wherein the posterior ocular disorder is wet age-related macular degeneration.

58. The method of claim 53, wherein the posterior ocular disorder is macular edema selected from cystoid macular edema and diabetic macular edema.

59. The method of claim 53, wherein the posterior ocular disorder is diabetic retinopathy or proliferative diabetic retinopathy.

60. The method of claim 51, wherein the ocular disorder is an anterior ocular disorder.

61. The method of claim 60, wherein the anterior ocular disorder is selected from glaucoma, allergic conjunctivitis, anterior uveitis, and cataracts.

62. The method of claim 60, wherein the anterior ocular disorder is glaucoma.

63. The method of claim 60, wherein the anterior ocular disorder is allergic conjunctivitis.

64. The method of claim 60, wherein the anterior ocular disorder is anterior uveitis.

65. The method of claim 60, wherein the anterior ocular disorder is cataracts.

66. The method of any one of claims 51-65, wherein the systemic administration of the compound of the compound of Formula II, or a pharmaceutically acceptable salt thereof, results in an accumulation of the compound of Formula II, or a pharmaceutically acceptable salt thereof, in a melanin-containing ocular tissue of the subject, forming a depot that provides extended delivery of the compound of Formula II to an ocular tissue of the subject for at least one week, two weeks, or three weeks after the administration.

67. The method of claim 66, wherein the melanin-containing ocular tissue is choroid-retina pigmented epithelium (C-RPE) and/or iris ciliary body (I-CB).

68. The method of any one of claims 51-67, wherein the compound of Formula II, or a pharmaceutically acceptable salt thereof, accumulates at a ratio of at least 2× in the choroidal tissue of the eye versus in the plasma such that a depot is formed in the choroidal tissue of the subject which provides extended delivery of the compound of Formula II, or a pharmaceutically acceptable salt thereof, for at least 7 days, two weeks, three weeks or one month, two months, three months, four months, five months, or six months after cessation of administration of the compound of Formula II, or a pharmaceutically acceptable salt thereof.

69. The method of any one of claims 51-67, wherein the compound of Formula II, or a pharmaceutically acceptable salt thereof, accumulates at a ratio of at least 2× in the iris-ciliary body of the eye versus in the plasma such that a depot is formed in the iris-ciliary body of the subject which provides extended delivery of the compound of Formula II, or a pharmaceutically acceptable salt thereof, for at least 7 days, two weeks, three weeks or one month, two months, three months, four months, five months, or six months after cessation of administration of the compound of Formula II, or a pharmaceutically acceptable salt thereof.

70. The method of any one of claims 51-69, wherein the compound of Formula II, or a pharmaceutically acceptable salt thereof, is administered orally or parenterally once or twice a day.

71. The method of claim 70, wherein the compound of Formula II, or a pharmaceutically acceptable salt thereof, is administered orally once a day in a tablet, capsule, gel cap, or other solid dosage form.

72. The method of any one of claims 51-71, wherein the compound of Formula II, or a pharmaceutically acceptable salt thereof, is provided in a ramp-up dosage for a first period of time and then a lower maintenance dosage for a second period of time.

73. The method of claim 72, wherein the first period of time is at least, or no greater than, 5, 6, 7, 8, 9, 10, 11, 12, 13, 14 or 15 days.

74. The method of claim 72, wherein the second period of time is at least 5, 10, 15, 20, or 25 days or 1, 2, 3, 4, 5, or 6 months.

75. The method of any one of claims 51-74, wherein the compound of Formula II is Compound 3: 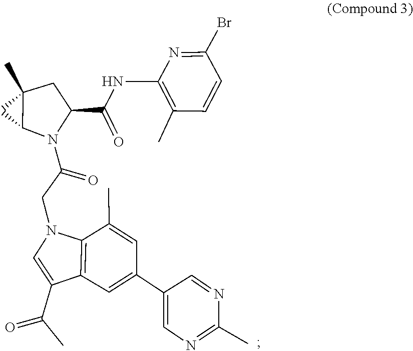 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]
or a pharmaceutically acceptable salt thereof.

76. The method of claim 75, wherein (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the retina of the eye compared to the plasma of the subject is maintained at no less than 0.60 throughout the dosing period.

77. The method of claim 76, wherein the dosing regimen results in a depot of Compound 3 in an eye of the subject by day 8, day 5, or day 3 of administration.

78. The method of any one of claims 75-77, wherein the ratio of Compound 3 in the retina of the eye compared to the plasma of the subject is maintained at no less than 0.65, no less than 0.70, or no less than 0.75 throughout the dosing period.

79. The method of claim 75, wherein (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the choroid-retina pigmented epithelium of the eye compared to the plasma is maintained at no less than 2.5× throughout the dosing period.

80. The method of claim 79, wherein the dosing regimen results in a depot of Compound 3 within an eye by day 8, by day 5, or by day 3 of administration.

81. The method of any one of claims 79-80, wherein the ratio of Compound 3 in the choroid-retina pigmented epithelium of the eye compared to the plasma is maintained between a range of 2.75 to 5.75 or greater throughout the dosing period.

82. The method of claim 75, wherein (a) the dosing regimen provides a plasma AUC₀₋₂₄ of between about 400 ng*hr/ml and 4,000 ng*hr/ml; (b) the dosing regimen results in a depot of Compound 3 within an eye of the subject by day 15 of administration, and (c) by day 15, the ratio of Compound 3 in the iris-ciliary body of the eye compared to the plasma of the subject is maintained at no less than 3 throughout the dosing period.

83. The method of claim 82, wherein by day 8, the ratio of Compound 3 in the iris-ciliary body compared to the plasma is maintained between a range of 3 to 5.5 or greater throughout the dosing period.

84. The method of any one of claims 75-83, the method comprising orally administering Compound 3, or a pharmaceutically acceptable salt thereof, at a dosing regimen that provides a plasma AUC₀₋₂₄ of between about 400 ng*hr/ml and 4,000 ng*hr/ml, wherein the dosing regimen results in a depot of Compound 3 within an eye of the subject by day 15 of administration, and wherein the depot contributes to the effective amount of Compound 3 contained in the eye throughout the dosing period.

85. The method of claim 84, wherein the dosing regimen results in a depot of Compound 3 within an eye by day 8 or by day 5 of administration.

86. The method of any one of claims 75-83, the method including orally administering Compound 3, or a pharmaceutically acceptable salt thereof, at a dosing regimen of from 20 mg to 350 mg, from 50 mg to 500 mg once a day.

87. The method of any one of claims 75-83, the method including orally administering Compound 3, or a pharmaceutically acceptable salt thereof, at a dosing regimen of less than 150 mg, less than 250 mg, or 150 mg once a day.

88. The method of any one of claims 1-87, wherein the depot if formed in the eye of the without the use of any injections, implants or a medical device into the eye of the subject.

89. The method of any one of claims 1-88, wherein accumulation of the compound is tested by radiolabeling or positron emission tomography.

90. The method of any of claims 1-89, wherein the method includes administering at least one additional active compound.

91. The method of claim 90, wherein the additional active compound is an anti-VEGF compound.

92. The method of claim 90, wherein the additional active compound is at least one complement 5 (C5) inhibitor.

93. The method of any of claims 1-92, wherein the subject does not suffer from a melanin deficiency.
